(19) United States
(12) Patent Application Publication
(10) Pub. No.: US 2024/0152048 A1
(43) Pub. Date: May 9, 2024

(54) RADIATION-SENSITIVE RESIN COMPOSITION AND PATTERN FORMATION METHOD

(71) Applicant: JSR CORPORATION, Tokyo (JP)

(72) Inventor: Ken MARUYAMA, Tokyo (JP)

(73) Assignee: JSR CORPORATION, Tokyo (JP)

(21) Appl. No.: 18/277,113

(22) PCT Filed: Dec. 20, 2021
(86) PCT No.: PCT/JP2021/046967

§ 371 Date: Aug. 14, 2023

(30) Foreign Application Priority Data
Mar. 19, 2021 (JP) 2021-045886

(51) Int. Cl. G03F 7/004; C07C 25/18; C07C 309/10; C07C 309/39; C07C 381/12; C07D 327/04; C08F 220/18; C08F 220/28; C08F 220/38; G03F 7/038; G03F 7/039

(52) U.S. Cl. CPC G03F 7/0045; C07C 25/18; C07C 309/10; C07C 309/39; C07C 381/12; C07D 327/04; C08F 220/1807; C08F 220/1812; C08F 220/283; C08F 220/382; G03F 7/038; G03F 7/039

(57) ABSTRACT

A radiation-sensitive resin composition includes: a resin including a structural unit represented by formula (1); at least one onium salt each including an organic acid anion moiety and an onium cation moiety; and a solvent. At least part of the organic acid anion moiety in the at least one onium salt includes an iodine-substituted aromatic ring structure. R is a hydrogen atom, an alkyl group having 1 to 5 carbon atoms, or a halogenated alkyl group having 1 to 5 carbon atoms, Y¹ is a divalent linking group, and X¹ is an acid-dissociable group, and n is 0 or 1. When n is 0, X¹ is represented by formula (s1) or (s2).

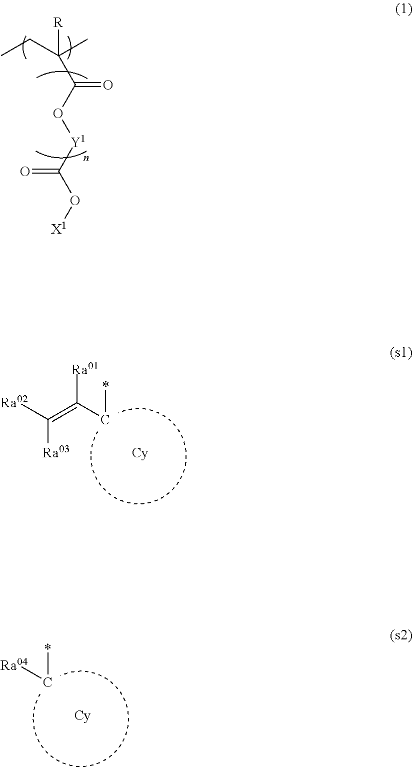

### TECHNICAL FIELD

The present invention relates to a radiation-sensitive resin composition and a pattern formation method.

### BACKGROUND ART

A photolithography technology using a resist composition has been used for the formation of a fine circuit in a semiconductor device. As a representative procedure, for example, a resist pattern is formed on a substrate by generating an acid by irradiating a coating film of the resist composition with radiation through a mask pattern, and then reacting in the presence of the acid as a catalyst to generate a difference in the solubility of a resin into an alkaline or organic solvent-based developer between an exposed area and an unexposed area.

In the photolithography technology, pattern miniaturization is promoted by using short-wavelength radiation, such as ArF excimer laser or by combining such radiation with an immersion exposure method (liquid immersion lithography). As a next-generation technology, further short-wavelength radiation, such as an electron beam, an X-ray, and an extreme ultraviolet ray (EUV) is being utilized, and a resist material containing an acid generator with a benzene ring having enhanced radiation absorption efficiency is also being studied (JP-A-2014-2359).

### PRIOR ART DOCUMENT

### Patent Document

Patent Document 1: JP-A-2014-2359

### SUMMARY OF THE INVENTION

### Problems to be Solved by the Invention

Even in the above-described next generation technology, various resist performances equivalent to or higher than conventional performances are required in sensitivity and critical dimension uniformity (CDU) performance, which is an index of uniformity of a line width and a hole diameter, and the like.

An object of the present invention is to provide a radiation-sensitive resin composition capable of forming a resist film having sensitivity and CDU performance at sufficient levels even when a next-generation technology is applied, and a method for forming a pattern.

### Means for Solving the Problems

As a result of intensive studies to solve the present problems, the present inventors have found that the above object can be achieved by adopting the following configurations, and have accomplished the present invention.

In one embodiment, the present invention relates to
a radiation-sensitive resin composition comprising:
a resin containing a structural unit represented by the following formula (1) (hereinafter, this structural unit is also referred to as a “structural unit (I)”);
one or two or more onium salts containing an organic acid anion moiety and an onium cation moiety; and
a solvent, wherein
at least part of the organic acid anion moiety in the onium salt contains an iodine-substituted aromatic ring structure.

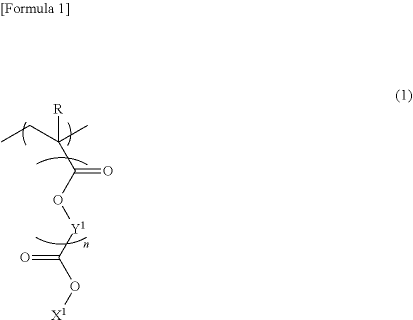

In the formula (1),
R is a hydrogen atom, an alkyl group having 1 to 5 carbon atoms, or a halogenated alkyl group having 1 to 5 carbon atoms, Y¹ is a divalent linking group, and X¹ is an acid-dissociable group, and
n is 0 or 1,
provided that when n is 0, X¹ is represented by the following formula (s1) or (s2).

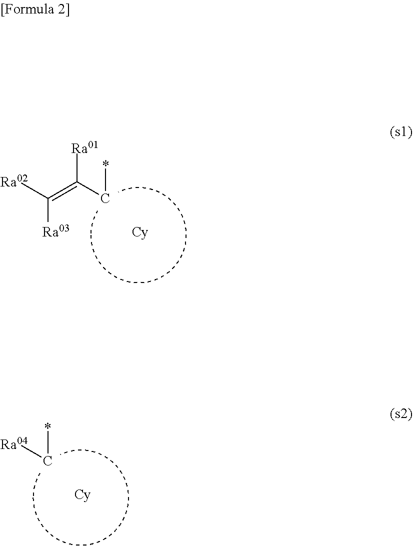

In the formula (s1),
Cy is an aliphatic cyclic group formed with a carbon atom,
Ra⁰¹ to Ra⁰³ each independently represent a hydrogen atom, a substituted or unsubstituted monovalent chain saturated hydrocarbon group having 1 to 10 carbon atoms, a substituted or unsubstituted monovalent aliphatic cyclic saturated hydrocarbon group having 3 to 20 carbon atoms, or an aliphatic cyclic structure formed by combining two or more of these groups with each other, provided that the aliphatic cyclic structure does not form a crosslinked structure;
in the formula (s2),
Cy has the same meaning as in the formula (s1), and
Ra⁰⁴ is a substituted or unsubstituted aromatic hydrocarbon group;
in the above formulas, both * each represent a bond with an oxygen atom.

With the radiation-sensitive resin composition, a resist film satisfying sensitivity and CDU performance can be constructed. The reason for this is not clear, but can be expected as follows. Absorption of radiation such as EUV having a wavelength of 13.5 nm by iodine atoms or fluorine atoms is very large, and this makes the radiation-sensitive resin composition highly sensitive. The iodine-substituted aromatic ring structure contained in at least part of the organic acid anion moiety in the onium salt can reduce acid diffusion owing to the largeness of the molecular weight of the iodine atom. It is presumed that the resist performance can be exhibited by the combination of these actions.

In another embodiment, the present invention relates to
a method for forming a pattern, the method comprising:
a step of directly or indirectly applying the radiation-sensitive resin composition on a substrate to form a resist film,
a step of exposing the resist film to light, and
a step of developing the exposed resist film with a developer.

Since the above-described radiation-sensitive resin composition superior in sensitivity and CDU performance is used in the method for forming a pattern, a high-quality resist pattern can efficiently be formed by the method.

### MODE FOR CARRYING OUT THE INVENTION

Hereinbelow, embodiments of the present invention will specifically be described, but the present invention is not limited to these embodiments.

<Radiation-Sensitive Resin Composition>

The radiation-sensitive resin composition (hereinafter also simply referred to as “composition”) according to the present embodiment contains one or two or more prescribed onium salts, and further contains a compound and a solvent. In addition, the composition contains a resin, as necessary. The composition may further contain other optional components as long as the effects of the present invention are not impaired. When the radiation-sensitive resin composition contains the prescribed onium salt and compound, the radiation-sensitive resin composition can be provided with high levels of sensitivity and CDU performance to a resulting resist film.

<Radiation-Sensitive Resin>

The radiation-sensitive resin composition (hereinafter also referred to simply as “resin”) is an assembly of polymers having a structural unit (I) (this resin is hereinafter also referred to as “base resin”). The base resin may contain, in addition to the structural unit (I), a structural unit having a phenolic hydroxy group or a structural unit that affords a phenolic hydroxy group by the action of an acid (hereinafter both of these are collectively referred to also as “structural unit (II)”), a structural unit (III) containing a lactone structure or the like, etc. Hereinbelow, each of the structural units will be described.

(Structural unit (I))

The structural unit (I) is represented by the following formula (1).

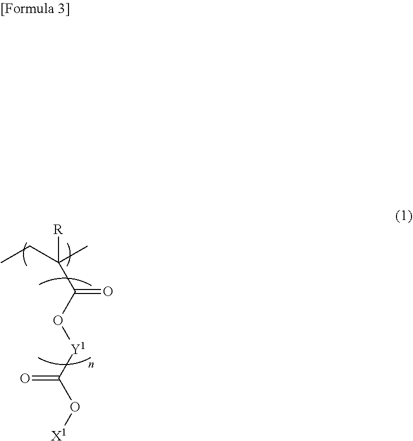

In the formula (1),
R is a hydrogen atom, an alkyl group having 1 to 5 carbon atoms, or a halogenated alkyl group having 1 to 5 carbon atoms, Y¹ is a divalent linking group, and X¹ is an acid-dissociable group, and
n is 0 or 1.

In the formula (1), the alkyl group having 1 to 5 carbon atoms represented by R is preferably a linear or branched alkyl group, and examples thereof include a methyl group, an ethyl group, a propyl group, an isopropyl group, a n-butyl group, an isobutyl group, a tert-butyl group, a pentyl group, an isopentyl group, and a neopentyl group.

In the formula (1), examples of the halogenated alkyl group having 1 to 5 carbon atoms represented by R include groups in which some or all of hydrogen atoms of the alkyl group having 1 to 5 carbon atoms are substituted with halogen atoms. Examples of the halogen atom include a fluorine atom, a chlorine atom, a bromine atom, and an iodine atom, and in particular, a fluorine atom is preferable.

The divalent linking group as Y¹ is not particularly limited, and suitable examples thereof include a divalent hydrocarbon group optionally having a substituent and a divalent linking group containing a hetero atom.

That a hydrocarbon group “has a substituent” means that some or all of the hydrogen atoms in the hydrocarbon group are substituted with a substituent (a group or an atom other than a hydrogen atom).

The hydrocarbon group may be either an aliphatic hydrocarbon group or an aromatic hydrocarbon group.

The aliphatic hydrocarbon group means a hydrocarbon group having no aromaticity.

The aliphatic hydrocarbon group as the divalent hydrocarbon group as Y¹ may be either saturated or unsaturated, and it is usually preferably saturated.

More specifically, examples of the aliphatic hydrocarbon group include a linear or branched aliphatic hydrocarbon group, and an aliphatic hydrocarbon group containing a ring in the structure.

The linear or branched aliphatic hydrocarbon group preferably has 1 to 10, more preferably 1 to 6, still more preferably 1 to 4, and most preferably 1 to 3 carbon atoms.

The linear aliphatic hydrocarbon group is preferably a linear alkylene group, and specifically, examples thereof include a methylene group [—CH₂— ], an ethylene group [—(CH₂)₂—], a trimethylene group [—(CH₂)₃ —], a tetramethylene group [—(CH₂)₄ —], and a pentamethylene group [—(CH₂)₅—].

The branched aliphatic hydrocarbon group is preferably a branched alkylene group, and specifically, examples thereof include alkylalkylene groups such as an alkylmethylene group such as —CH(CH₃)—, —CH(CH₂CH₃)—, —C(CH₃)₂—, —C(CH₃) (CH₂CH₃)—, —C(CH₃) (CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃) CH₂CH₂— and —CH₂CH(CH₃) CH₂—; and alkyltetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

The linear or branched aliphatic hydrocarbon group may or may not have a substituent.

Examples of the aliphatic hydrocarbon group containing a ring in the structure include an alicyclic hydrocarbon group (a group formed by removing two hydrogen atoms from an aliphatic hydrocarbon ring), a group in which an alicyclic hydrocarbon group is bonded to a terminal of a linear or branched aliphatic hydrocarbon group, and a group in which an alicyclic hydrocarbon group is interposed in the middle of a linear or branched aliphatic hydrocarbon group. Examples of the linear or branched aliphatic hydrocarbon group include the same groups as those recited above.

The alicyclic hydrocarbon group preferably has 3 to 20, and more preferably 3 to 12 carbon atoms.

The alicyclic hydrocarbon group may be either polycyclic or monocyclic. The monocyclic alicyclic hydrocarbon group is preferably a group obtained by removing two hydrogen atoms from a monocycloalkane. The monocycloalkane is preferably one having 3 to 6 carbon atoms, and specifically, examples thereof include cyclopentane and cyclohexane. The polycyclic alicyclic hydrocarbon group is preferably a group obtained by removing two hydrogen atoms from a polycycloalkane, and the polycycloalkane is preferably one having 7 to 12 carbon atoms, and specifically, examples thereof include adamantane, norbornane, isobornane, tricyclodecane, and tetracyclododecane.

The alicyclic hydrocarbon group may or may not have a substituent.

The aromatic hydrocarbon group is a hydrocarbon group having an aromatic ring.

The aromatic hydrocarbon group as the divalent hydrocarbon group in Y¹ preferably has 3 to 30, more preferably 5 to 30, still more preferably 5 to 20, particularly preferably 6 to 15, and most preferably 6 to 10 carbon atoms. However, the above-mentioned number of carbon atoms does not include the number of carbon atoms in the substituent.

Specifically, examples of the aromatic ring of the aromatic hydrocarbon group include an aromatic hydrocarbon ring such as benzene, biphenyl, fluorene, naphthalene, anthracene, and phenanthrene; and an aromatic heterocyclic ring in which some of the carbon atoms constituting the aromatic hydrocarbon ring are replaced by a hetero atom. Examples of the hetero atom in the aromatic heterocyclic ring include an oxygen atom, a sulfur atom, and a nitrogen atom.

Specifically, examples of the aromatic hydrocarbon group include a group (arylene group) obtained by removing two hydrogen atoms from the aromatic hydrocarbon ring; and a group in which one of the hydrogen atoms of a group (aryl group) obtained by removing one hydrogen atom from the aromatic hydrocarbon ring is substituted with an alkylene group (e.g., a group obtained by further removing one hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The number of carbon atoms of the alkylene group (the alkyl chain in the arylalkyl group) is preferably 1 to 4, more preferably 1 to 2, and particularly preferably 1.

The aromatic hydrocarbon group may or may not have a substituent.

The hetero atom in the “divalent linking group containing a hetero atom” as Y¹ is an atom other than a carbon atom and a hydrogen atom, and examples thereof include an oxygen atom, a nitrogen atom, a sulfur atom, and a halogen atom.

Examples of the divalent linking group containing a hetero atom include —O—, —C(═O)—O—, —C(═O)—, —O—C(═O)—O—, —C(═O)—NH—, —NH— (wherein H may be substituted with a substituent such as an alkyl group or an acyl group), —S—, —S(═O)₂—, —S(═O)₂—O—, —NH—C(═O)—, ═N—, a group represented by the general formula —Y²¹—O—Y²²—, —[Y²¹—C(═O)—O]ₘₚ, —Y²²—, or —Y²¹—O—C(═O)—Y²²—[wherein Y²¹ and Y²² are each independently a divalent hydrocarbon group optionally having a substituent, O is an oxygen atom, and mp is an integer of 0 to 3].

When Y¹ is —NH—, H thereof may be substituted with a substituent such as an alkyl group or an aryl group (aromatic group). Y²¹ and Y²² are each independently a divalent hydrocarbon group optionally having a substituent. Examples of the divalent hydrocarbon group include the same groups as those recited above as the “divalent hydrocarbon group optionally having a substituent” as Y¹.

As Y²¹, a linear aliphatic hydrocarbon group is preferable, a linear alkylene group is more preferable, a linear alkylene group having 1 to 5 carbon atoms is still more preferable, and a methylene group or an ethylene group is particularly preferable.

As Y²², a linear or branched aliphatic hydrocarbon group is preferable, and a methylene group, an ethylene group, or an alkylmethylene group is more preferable.

The divalent linking group containing a hetero atom is preferably a linear chain group having an oxygen atom as a hetero atom, for example, a group containing an ether linkage or an ester linkage, and more preferably a group represented by the above formula —Y²¹—O—Y²²—, —[Y²¹—C(═O)—O]ₘₚ—Y²²—, or —Y²¹—O—C(═O)—Y²²—.

Among the above, the divalent linking group as Y¹ is particularly preferably a linear or branched alkylene group, a divalent alicyclic hydrocarbon group, or a divalent linking group containing a hetero atom. Among these, the linear or branched alkylene group or the divalent linking group containing a hetero atom is preferable.

In the formula (1), the acid-dissociable group represented by X¹ is a group having an acid-dissociable property with which at least a bond between the acid-dissociable group and an atom adjacent to the acid-dissociable group can be cleaved by the action of an acid.

The acid-dissociable group is not particularly limited, and a group that forms a cyclic or chain tertiary alkyl ester with a carboxy group in (meth)acrylic acid or the like; an acetal type acid-dissociable group such as alkoxyalkyl group; etc. are widely known.

Herein, the “tertiary alkyl ester” refers to a structure in which the hydrogen atom of a carboxy group is substituted with a chain or cyclic alkyl group and forms an ester, and the tertiary carbon atom of the chain or cyclic alkyl group is bonded to an oxygen atom at a terminal of the carbonyloxy group (—C(═O)—O—). In the tertiary alkyl ester, when an acid acts, the bond is cleaved between the oxygen atom and the tertiary carbon atom and a carboxy group is formed.

The chain or cyclic alkyl group may have a substituent.

Hereinafter, a group that is acid-dissociable due to the constitution of a tertiary alkyl ester with a carboxy group is referred to as a “tertiary alkyl ester type acid-dissociable group” for convenience.

Examples of the tertiary alkyl ester type acid-dissociable group include an aliphatic branched chain acid-dissociable group and an acid-dissociable group containing an aliphatic cyclic group.

Herein, the term “aliphatic branched chain” means to have a branched chain structure having no aromaticity. The structure of the “aliphatic branched chain acid-dissociable group” is not limited to a group composed of carbon and hydrogen (hydrocarbon group), but is preferably a hydrocarbon group. The “hydrocarbon group” may be either saturated or unsaturated, but is usually preferably saturated.

Examples of the aliphatic branched chain acid-dissociable group include a group represented by —C(R⁷¹)(R⁷²)(R⁷³). In the formula, R⁷¹ to R⁷³ are each independently a linear alkyl group having 1 to 5 carbon atoms. The group represented by -0(R⁷¹) (R⁷²) (R⁷³) preferably has 4 to 8 carbon atoms, and specifically, examples thereof include a tert-butyl group, a 2-methyl-2-butyl group, a 2-methyl-2-pentyl group, and a 3-methyl-3-pentyl group. In particular, a tert-butyl group is preferable.

The “aliphatic cyclic group” refers to a monocyclic or polycyclic group having no aromaticity.

The aliphatic cyclic group in the “acid-dissociable group containing an aliphatic cyclic group” may or may not have a substituent.

The structure of the basic ring of the aliphatic cyclic group excluding the substituent is not limited to a group composed of carbon and hydrogen (hydrocarbon group), but is preferably a hydrocarbon group. In addition, the hydrocarbon group may be either saturated or unsaturated, but is usually preferably saturated.

The aliphatic cyclic group may be either monocyclic or polycyclic.

Examples of the aliphatic cyclic group include a group obtained by removing one or more hydrogen atoms from a monocycloalkane; and a group obtained by removing one or more hydrogen atoms from a polycycloalkane such as a bicycloalkane, a tricycloalkane, and a tetracycloalkane. In addition, a group in which some of the carbon atoms constituting the ring of these alicyclic hydrocarbon groups are replaced by an ether linkage (—O—) is also included.

Examples of the acid-dissociable group containing an aliphatic cyclic group include groups represented by the following formulas (1-1) to (1-9) and groups represented by the following formulas (2-1) to (2-6).

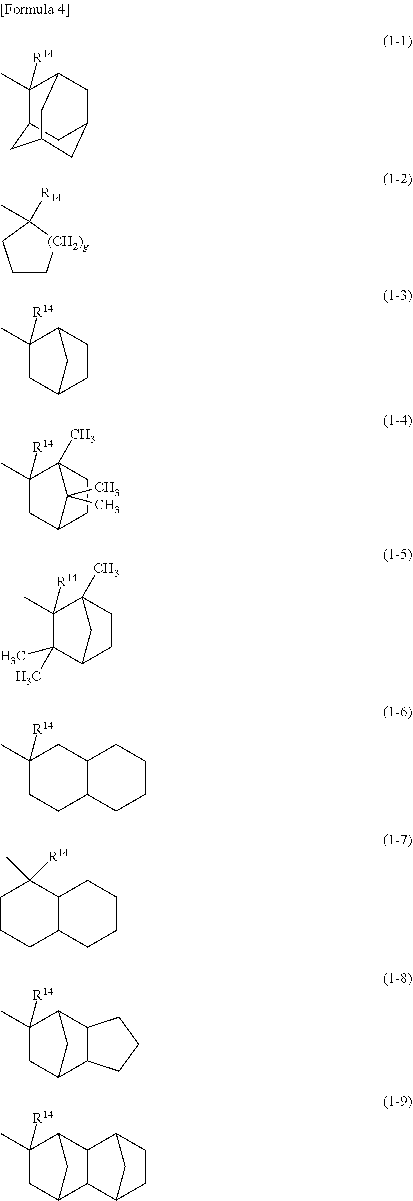

Wherein R¹⁴ is an alkyl group and g is an integer of 0 to 8.

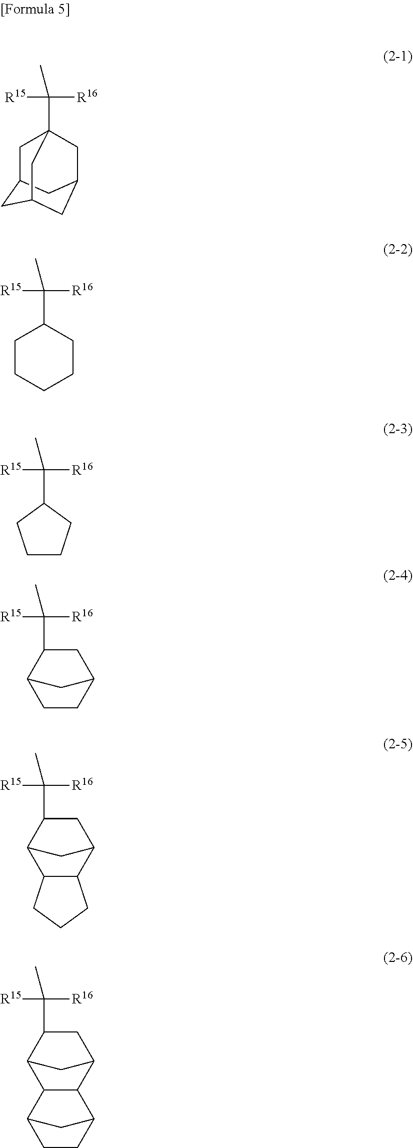

Wherein R¹⁵ and R¹⁶ are each independently an alkyl group.

In the formulas (1-1) to (1-9), the alkyl group as R¹⁴ may be linear, branched, or cyclic, and is preferably linear or branched.

The linear alkyl group preferably has 1 to 5, more preferably 1 to 4, and still more preferably 1 or 2 carbon atoms.

The branched alkyl group preferably has 3 to 10, and more preferably 3 to 5 carbon atoms.

Examples of the cyclic alkyl group include the same as those to the aliphatic cyclic group.

g is preferably an integer of 0 to 4, more preferably an integer of 1 to 4, and still more preferably 1, 2, or 4.

In the formulas (2-1) to (2-6), examples of the alkyl group as R¹⁵ to R¹⁶ include the same as those of the alkyl group as R¹⁴.

In the above formulas (1-1) to (1-9) and (2-1) to (2-6), some of the carbon atoms constituting each of the rings may be replaced by an etheric oxygen atom (—O—).

Generally, the “acetal type acid-dissociable group” replaces a hydrogen atom at the terminal of an OH-containing polar group such as a carboxy group and a hydroxy group and is bonded to an oxygen atom. Then, when an acid acts, a bond is cleaved between the acetal type acid-dissociable group and the oxygen atom to which the acetal type acid-dissociable group is bonded, thereby forming an OH-containing polar group such as a carboxy group or a hydroxy group.

In the above formula (1), n is 0 or 1.

X¹ in the formula (1) is, besides the above-described acid-dissociable group, preferably represented by the following formula (s1) or (s2). It is noted that when n is 0, X¹ in the formula (1) is represented by the following formula (s1) or (s2).

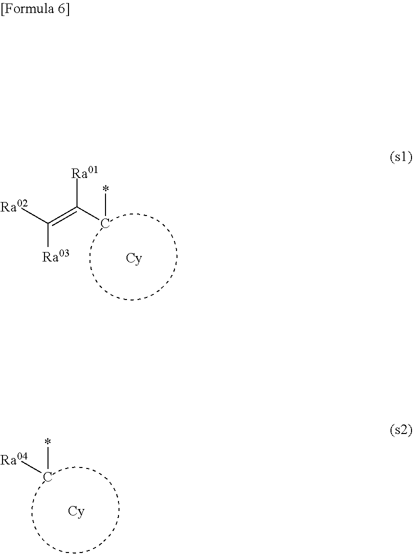

In the formula (s1),
Cy is an aliphatic cyclic group formed with a carbon atom,
Ra⁰¹ to Ra⁰³ each independently represent a hydrogen atom, a substituted or unsubstituted monovalent chain saturated hydrocarbon group having 1 to 10 carbon atoms, a substituted or unsubstituted monovalent aliphatic cyclic saturated hydrocarbon group having 3 to 20 carbon atoms, or an aliphatic cyclic structure formed by combining two or more of these groups with each other, provided that the aliphatic cyclic structure does not form a crosslinked structure;
in the formula (s2),
Cy has the same meaning as in the formula (s1), and
Ra⁰⁴ is a substituted or unsubstituted aromatic hydrocarbon group;
in the above formulas, both * each represent a bond with an oxygen atom.

The aliphatic cyclic group represented by Cy may be either a monocyclic group or a polycyclic group. Examples of the monocyclic aliphatic cyclic group include a group obtained by removing one or more hydrogen atoms from a monocycloalkane. The monocycloalkane is preferably one having 3 to 6 carbon atoms, and specifically, examples thereof include cyclopentane and cyclohexane. Examples of the polycyclic aliphatic cyclic group include a group obtained by removing one or more hydrogen atoms from a polycycloalkane. Among them, the monocyclic aliphatic cyclic group is preferable, and a group obtained by removing one or more hydrogen atoms from cyclopentane or cyclohexane is more preferable.

Some or all of the hydrogen atoms of the aliphatic cyclic group may be substituted.

In the formula (s1), examples of the monovalent chain saturated hydrocarbon group having 1 to 10 carbon atoms as Ra⁰¹ to Ra⁰³ include an alkyl group having 1 to 10 carbon atoms.

Examples of the monovalent aliphatic cyclic saturated hydrocarbon group having 3 to 20 carbon atoms as Ra⁰¹ to Ra⁰³ include a monocyclic aliphatic saturated hydrocarbon group and a polycyclic aliphatic saturated hydrocarbon group.

As Ra⁰¹ to Ra⁰³, a hydrogen atom is particularly preferable from the viewpoint of ease of synthesis of a monomer compound from which the structural unit (I) is derived.

Examples of the substituent of the chain saturated hydrocarbon group or the aliphatic cyclic saturated hydrocarbon group represented by Ra⁰¹ to Ra⁰³ include the same groups as those as Ra⁰⁵ described above.

Examples of a group containing a carbon-carbon double bond generated through the formation of a cyclic structure by bonding two or more of Ra⁰¹ to Ra⁰³ to each other include a cyclopentenyl group, a cyclohexenyl group, a methylcyclopentenyl group, a methylcyclohexenyl group, a cyclopentylideneethenyl group, and a cyclohexylideneethenyl group.

The aliphatic cyclic group having no crosslinked structure represented by Cy in the formula (s2) is the same as the aliphatic cyclic group represented by Cy in the formula (s1).

In the formula (s2), examples of the aromatic hydrocarbon group as Ra⁰⁴ include a group obtained by removing one or more hydrogen atoms from an aromatic hydrocarbon ring having 6 to 30 carbon atoms. In particular, Ra⁰⁴ is preferably a group obtained by removing one or more hydrogen atoms from an aromatic hydrocarbon ring having 6 to 15 carbon atoms, and most preferably a group obtained by removing one or more hydrogen atoms from benzene.

Specific examples of the acid-dissociable group represented by the above formula (s1) are as follows. * represents a bond.

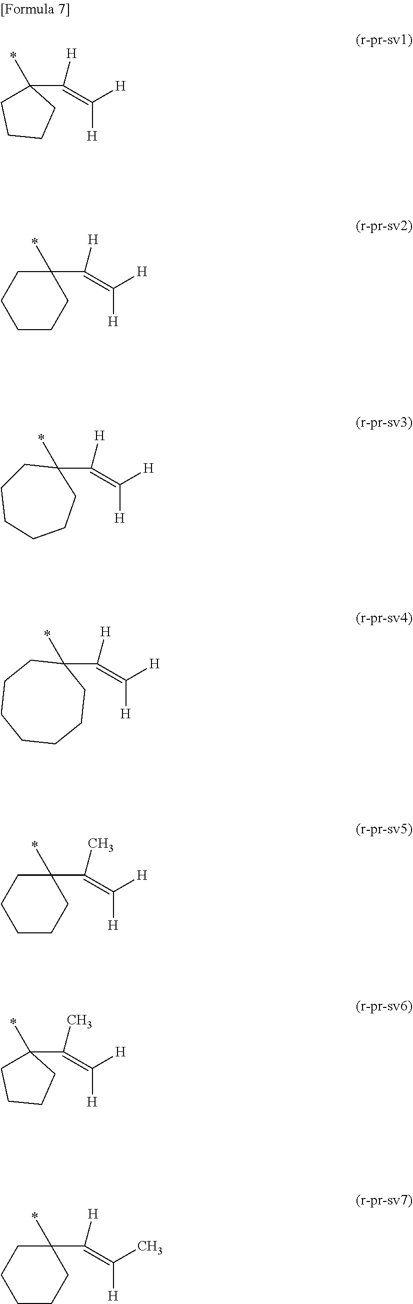

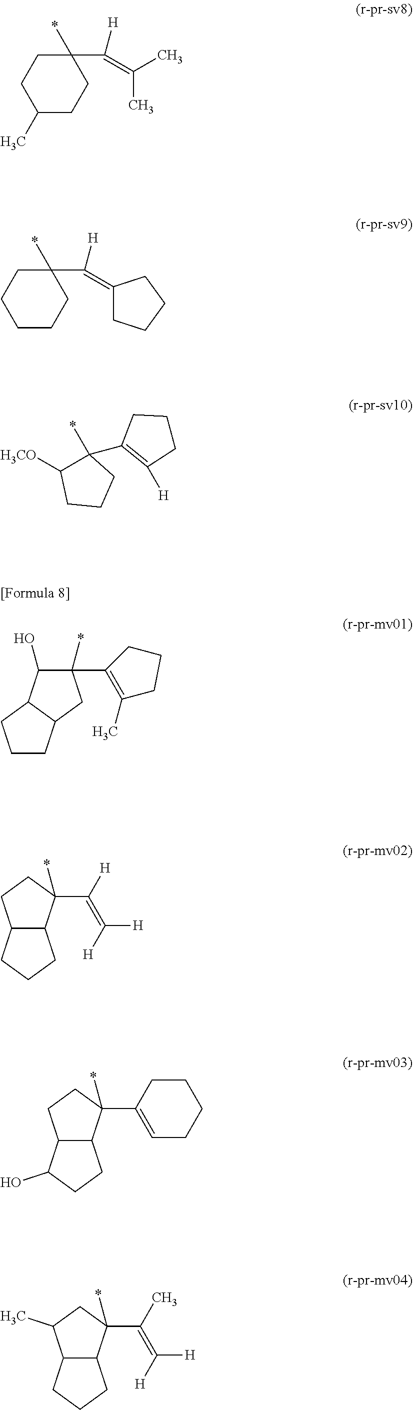

Specific examples of the acid-dissociable group represented by the above formula (s2) are as follows. * represents a bond.

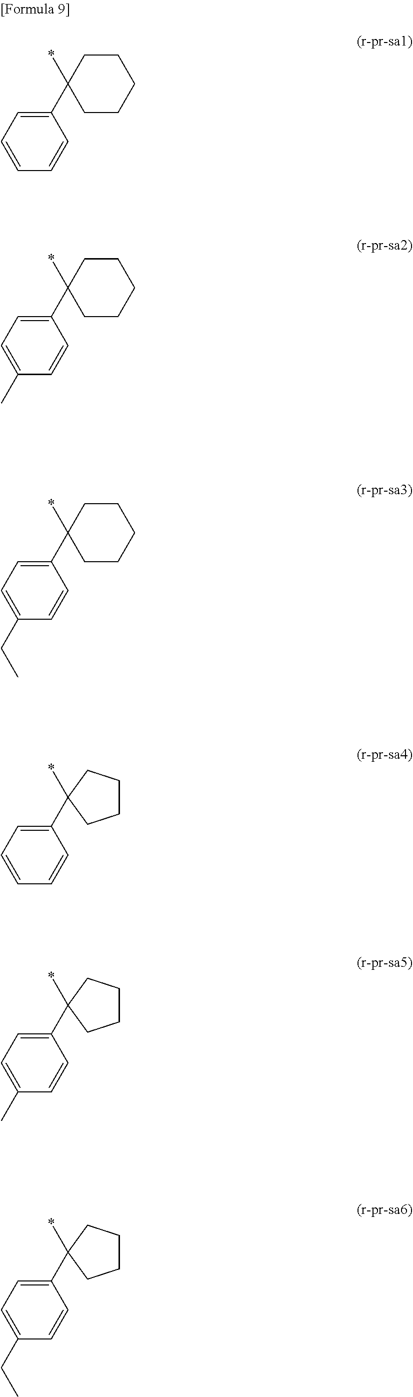 [SMILES: *C1(c2ccccc2)CCCCC1 ; *C1(c2ccc(C)cc2)CCCCC1 ; *C1(c2ccc(CC)cc2)CCCCC1 ; *C1(c2ccccc2)CCCC1 ; *C1(c2ccc(C)cc2)CCCC1 ; *C1(c2ccc(CC)cc2)CCCC1]

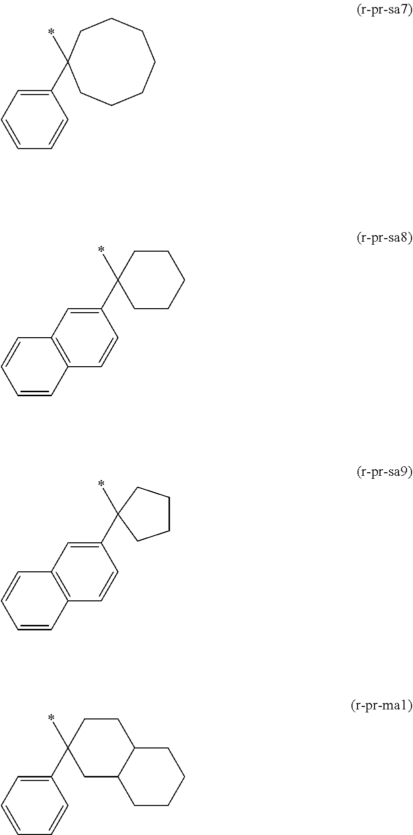 [SMILES: *C1(c2ccccc2)CCCCCCC1 ; *C1(c2ccc3ccccc3c2)CCCCC1 ; *C1(c2ccc3ccccc3c2)CCCC1 ; *C1(c2ccccc2)CCC2CCCCC2C1]

Specific examples of the structural unit represented by the above formula (1) are shown below. In each formula, R^{α} represents a hydrogen atom, a methyl group, or a trifluoromethyl group.

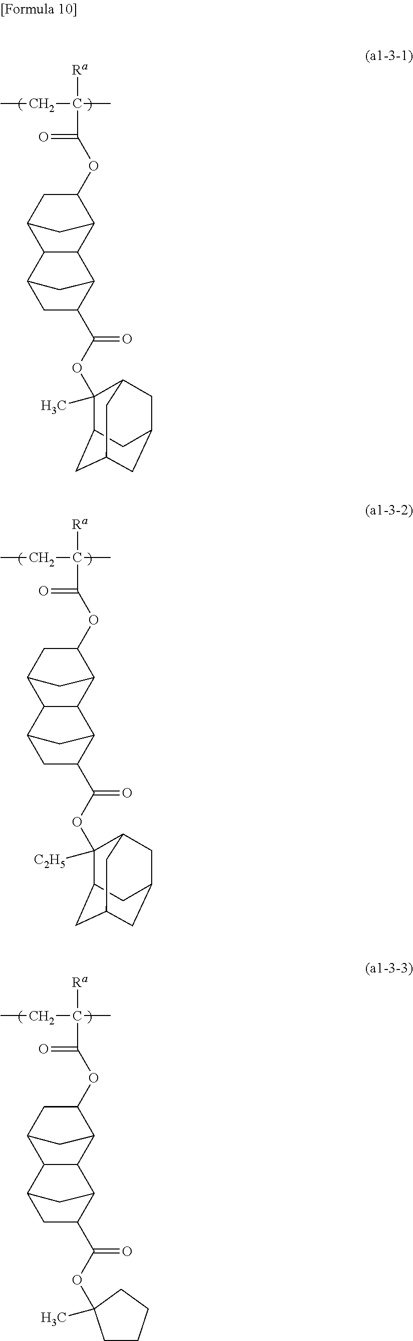

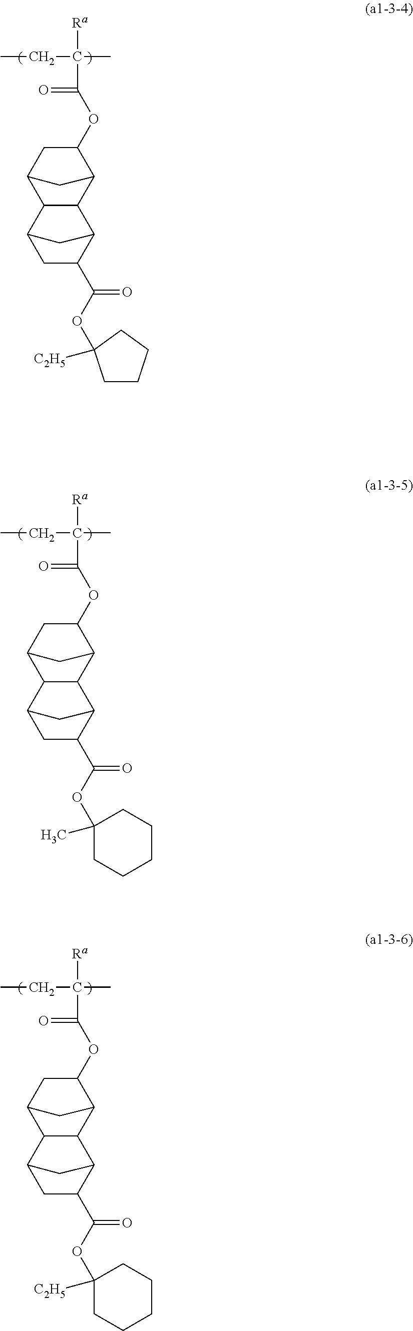

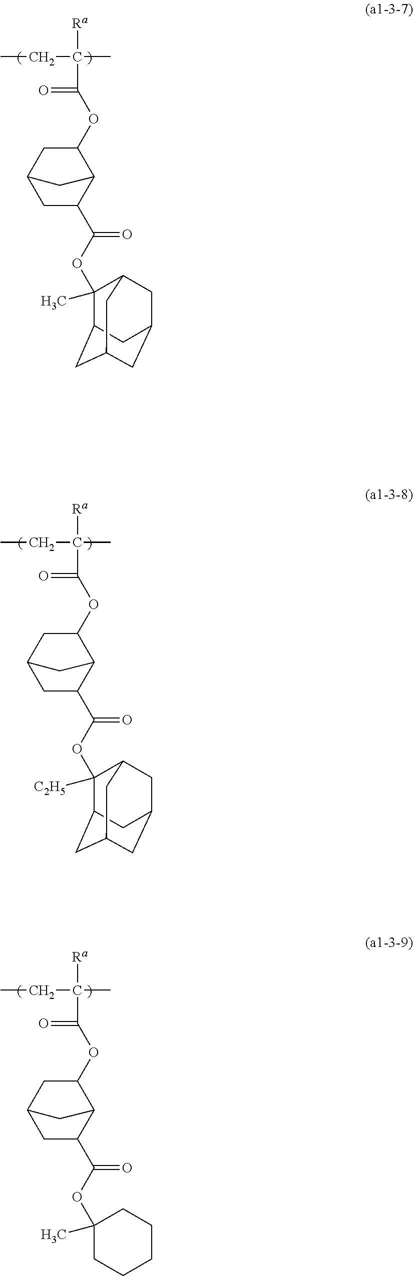

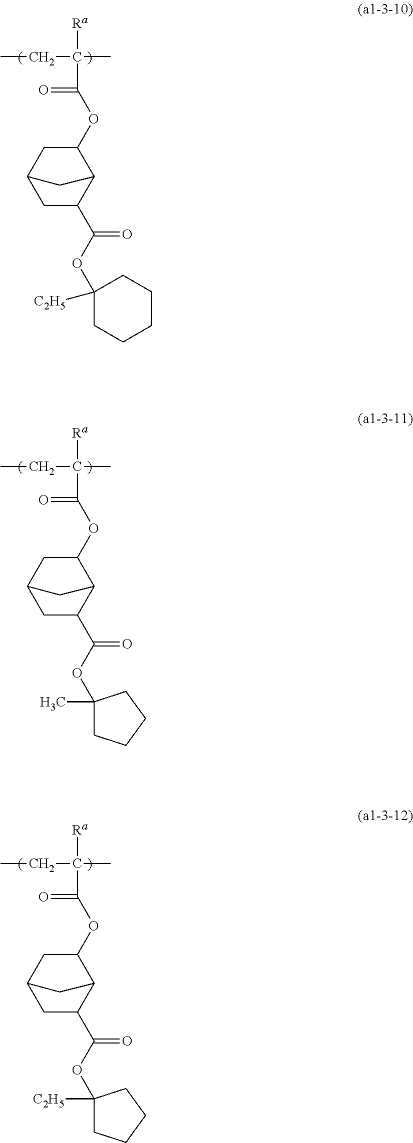

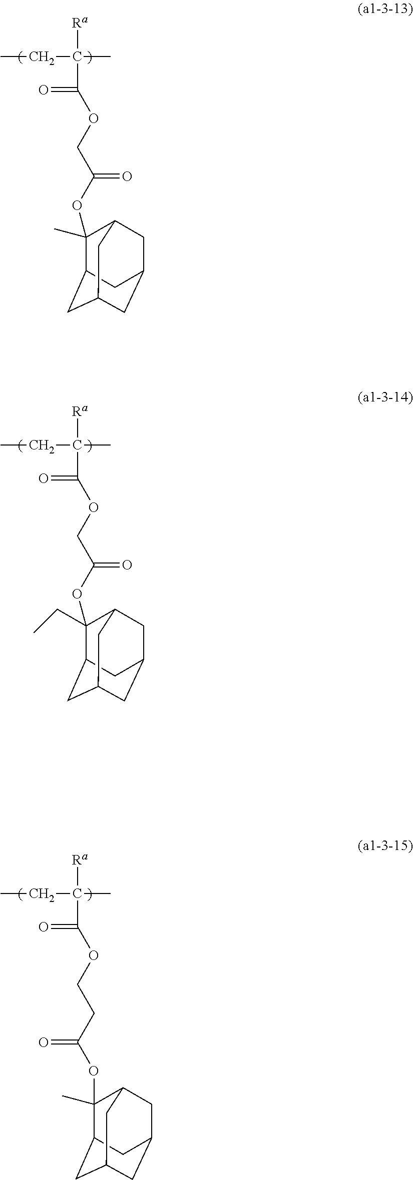

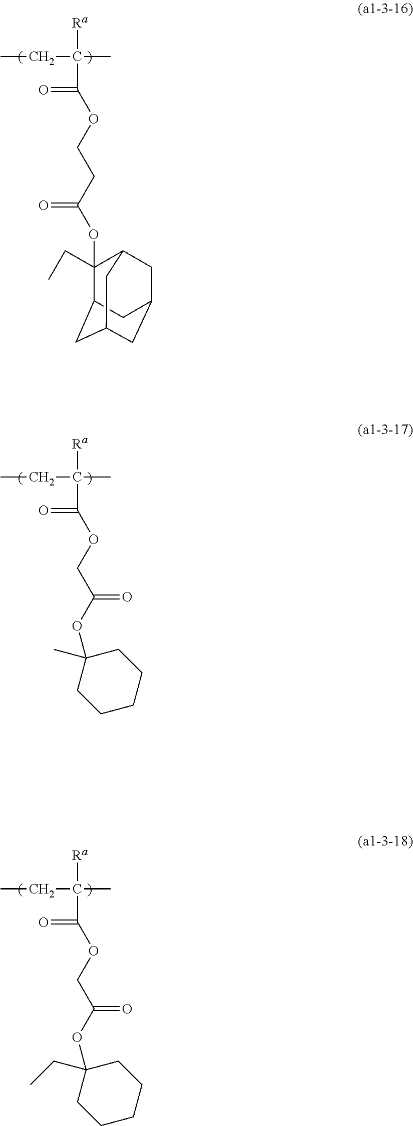

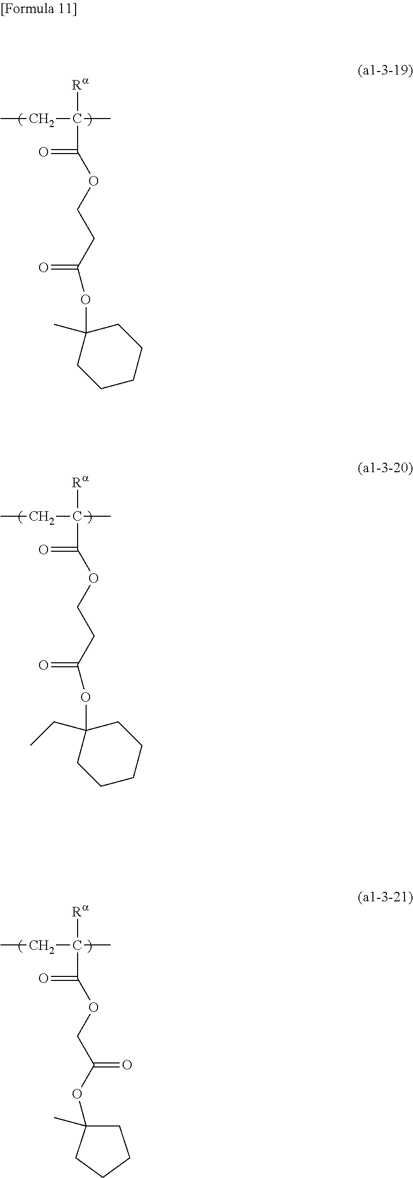

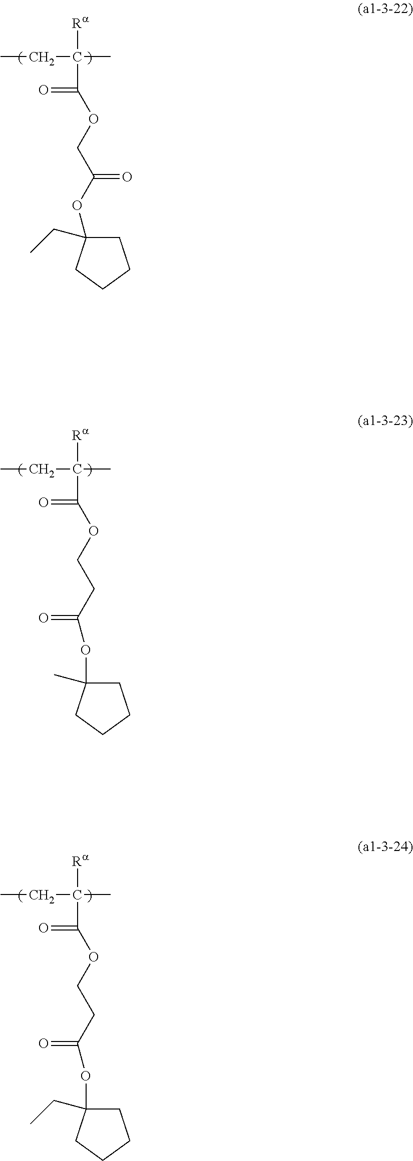

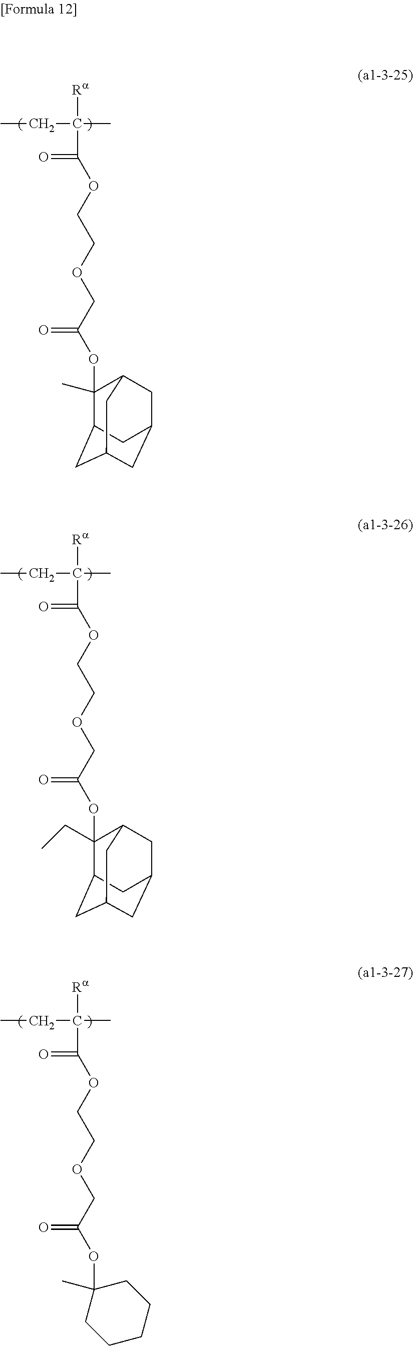

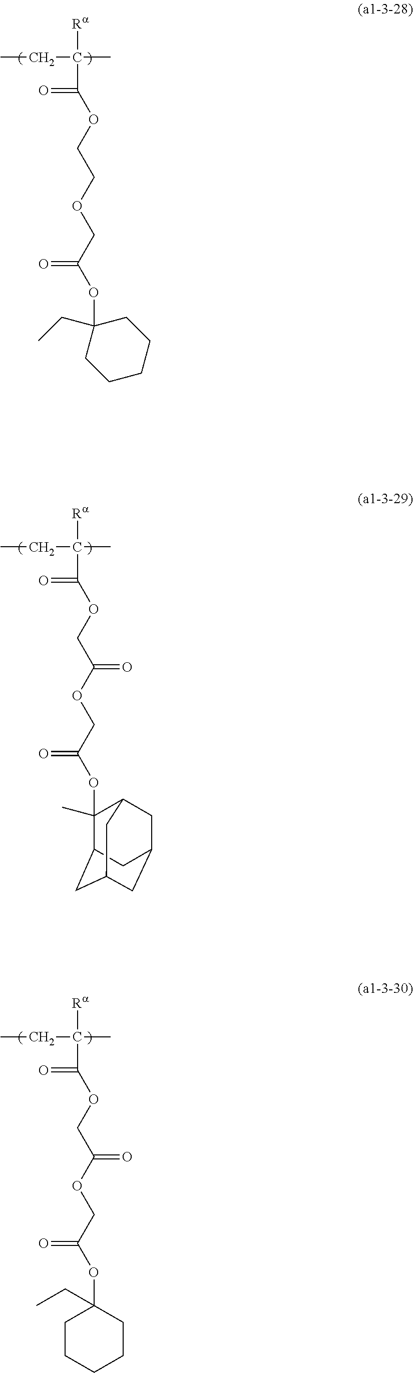

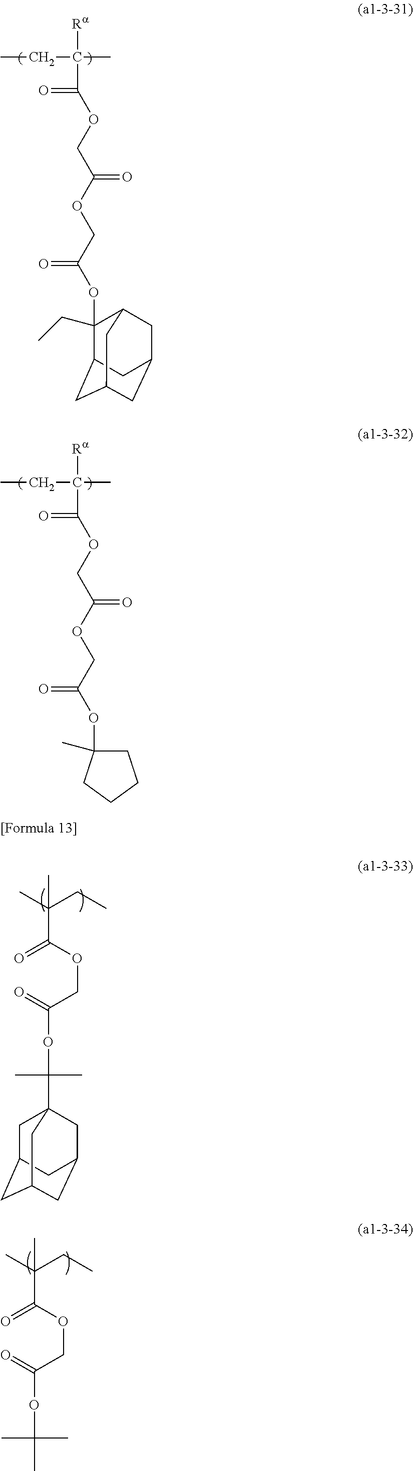

Specific examples of the structural unit (I) having an acid-dissociable group represented by the formula (s1) or (s2) are shown below. In the following formula, R^{α} represents a hydrogen atom, a methyl group, or a trifluoromethyl group.

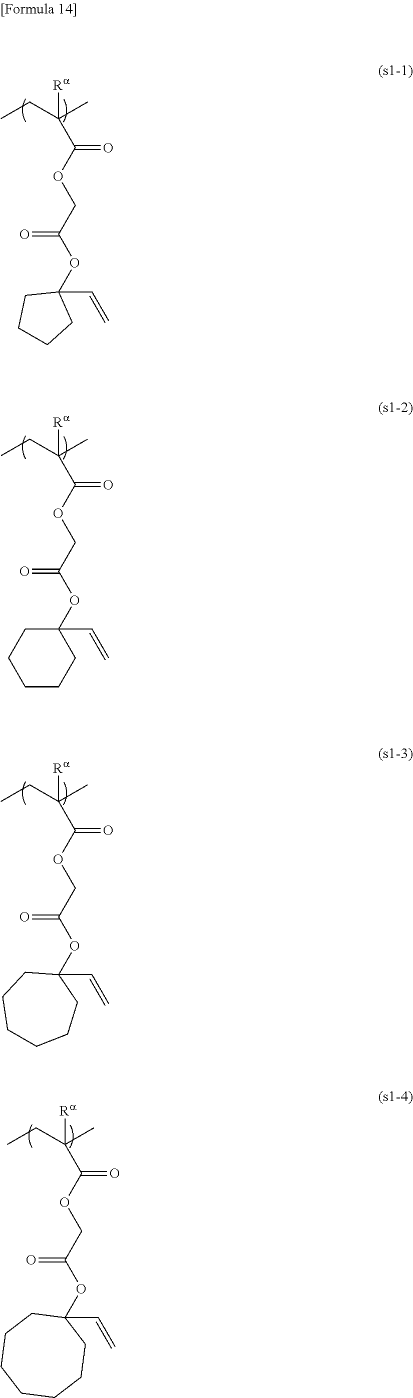

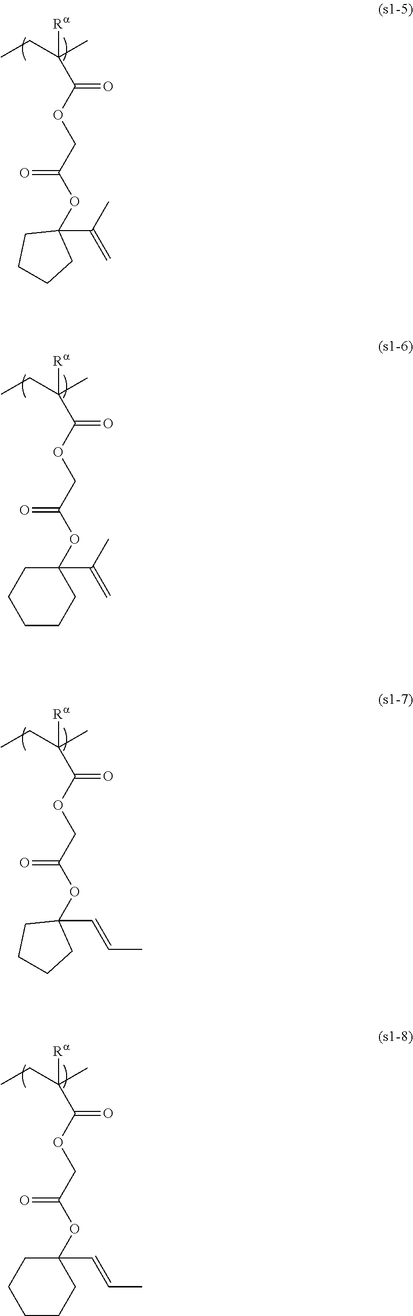

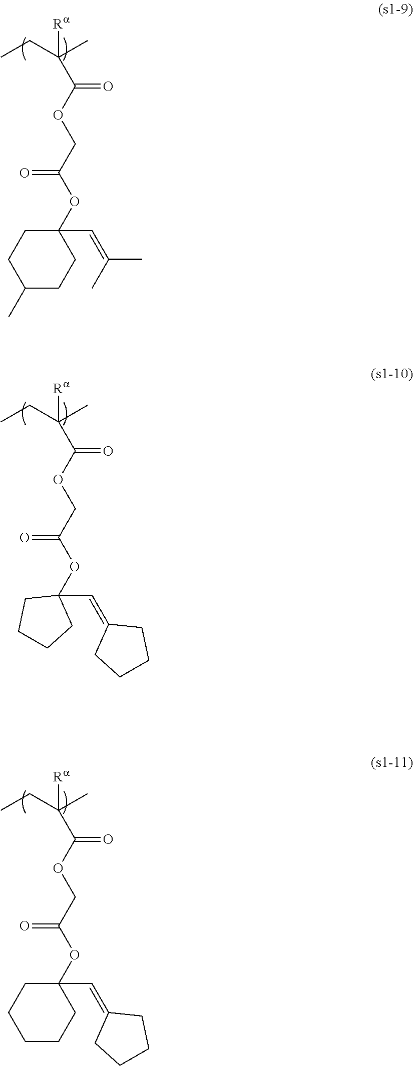

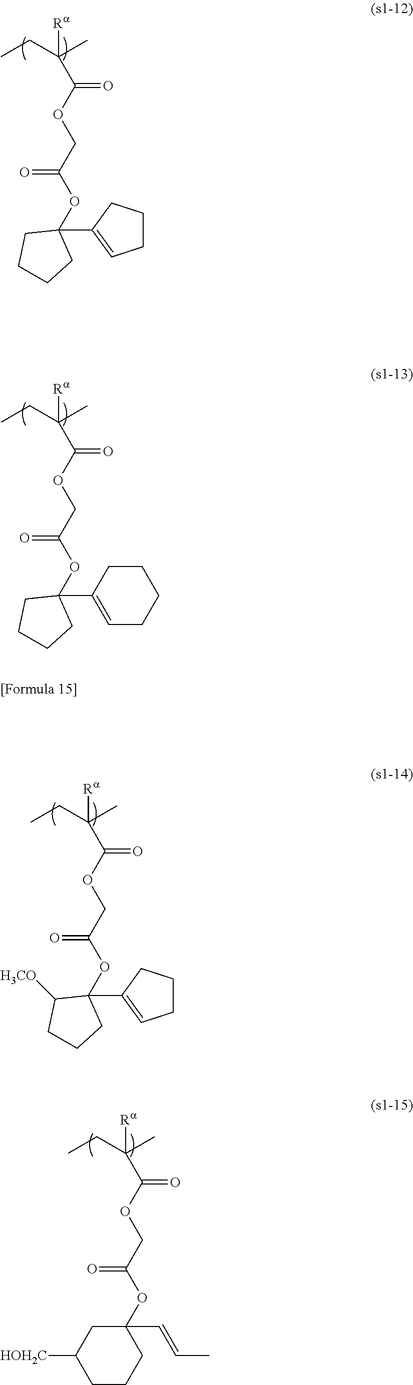

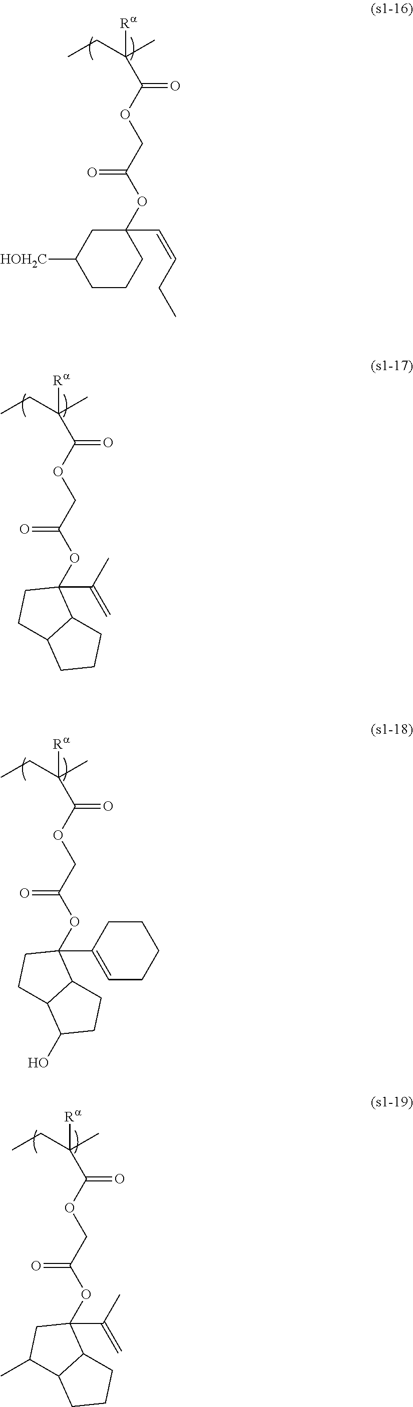

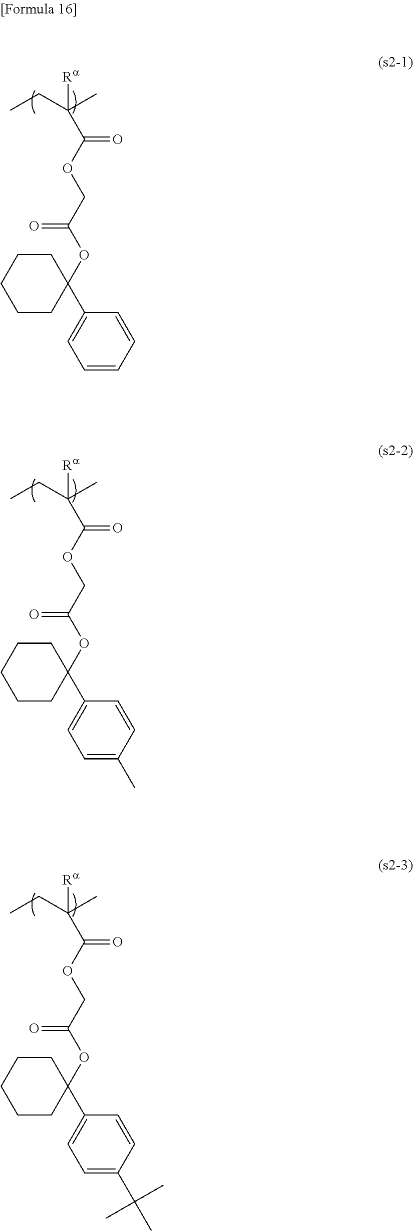

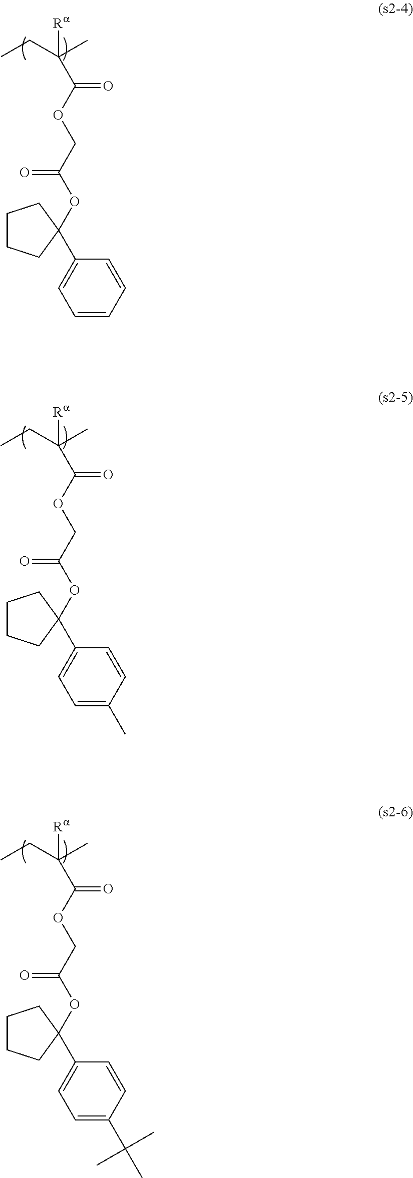

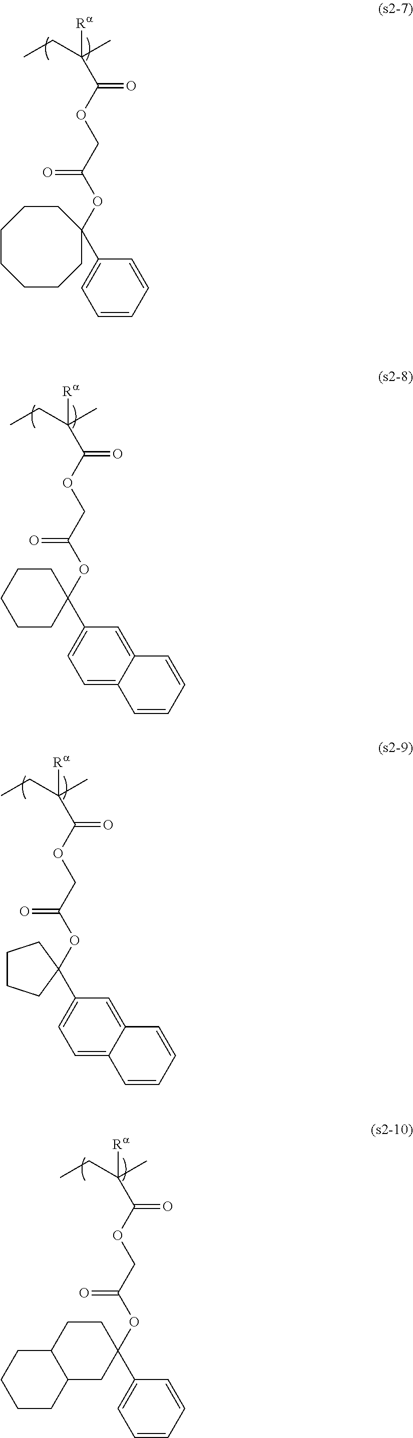

Among the above examples, the structural unit (I) is preferably at least one selected from the group consisting of constitutional units represented by the above formulas (a1-3-13) to (a1-3-24), (a1-3-33) to (a1-3-34), formulas (s1-1) to (s1-4), and formulas (s2-1) to (s1-6).

The content of the structural unit (I) in the resin (when there are a plurality of types of structural unit (I), a total content thereof is taken) is preferably 10 mol % or more, more preferably 20 mol % or more, and still more preferably 30 mol % or more based on all structural units constituting the resin. The content is preferably 70 mol % or less, more preferably 60 mol % or less, and still more preferably 50 mol % or less. When the content of the structural unit (I) is adjusted to within the above range, the sensitivity and CDU performance of the radiation-sensitive resin composition can be further improved.

(Structural unit (II))

The structural unit (II) is a structural unit having a phenolic hydroxy group or a structural unit that affords a phenolic hydroxy group due to the action of an acid. In the present invention, a phenolic hydroxy group generated through deprotection due to the action of an acid generated by exposure to light is also included as the phenolic hydroxy group of the structural unit (II). When the resin contains the structural unit (II), the solubility thereof in a developer can be more appropriately adjusted, and as a result, the sensitivity and the like of the radiation-sensitive resin composition can be further improved. When KrF excimer laser light, EUV, electron beam or the like is used as radiation to be applied in an exposure step in a method for forming a resist pattern, the structural unit (II) contributes to improvement in etching resistance and improvement in the difference in solubility in a developer (namely, dissolution contrast) between an exposed area and an unexposed area. In particular, the resin can be suitably applied to pattern formation using exposure with radiation having a wavelength of 50 nm or less such as electron beam or EUV. The structural unit (II) is preferably represented by the following formula (2).

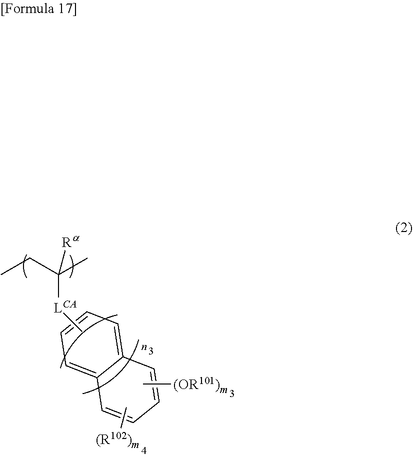

In the formula (2),
R^{α} is a hydrogen atom, a fluorine atom, a methyl group, or a trifluoromethyl group;
L^{CA} is a single bond, —COO—* or —O—; * is a bond on the aromatic ring side;
R¹⁰¹ is a hydrogen atom or a protective group that is deprotected by the action of an acid; when there are a plurality of R¹⁰¹'s, the plurality of R¹⁰¹'s are the same or different from each other;
R¹⁰² is a cyano group, a nitro group, an alkyl group, a fluorinated alkyl group, an alkoxycarbonyloxy group, an acyl group, or an acyloxy group; when there are a plurality of R¹⁰²'s, the plurality of R¹⁰²'s are the same or different from each other;
n₃ is an integer of 0 to 2, m₃ is an integer of 1 to 8, and m₄ is an integer of 0 to 8, provided that 1≤m₃+m₄≤2n₃+5 is satisfied.

The R^{α} is preferably a hydrogen atom or a methyl group from the viewpoint of the copolymerizability of a monomer that affords the structural unit (II).

L^{CA} is preferably a single bond or —COO—*.

Examples of the protecting group that is deprotected by the action of an acid represented by R¹⁰¹ include the same groups as the acid-dissociable group as X¹ in the formula (1).

Examples of the alkyl group in R¹⁰² include linear or branched alkyl groups having 1 to 8 carbon atoms such as a methyl group, an ethyl group, and a propyl group. Examples of the fluorinated alkyl group include linear or branched fluorinated alkyl groups having 1 to 8 carbon atoms such as a trifluoromethyl group and a pentafluoroethyl group. Examples of the alkoxycarbonyloxy group include chain or alicyclic alkoxycarbonyloxy groups having 2 to 16 carbon atoms such as a methoxycarbonyloxy group, a butoxycarbonyloxy group, and an adamantylmethyloxycarbonyloxy group. Examples of the acyl group include aliphatic or aromatic acyl groups having 2 to 12 carbon atoms such as an acetyl group, a propionyl group, a benzoyl group, and an acryloyl group. Examples of the acyloxy group include aliphatic or aromatic acyloxy groups having 2 to 12 carbon atoms such as an acetyloxy group, a propionyloxy group, a benzoyloxy group, and an acryloyloxy group.

The n₃ is preferably 0 or 1, and more preferably 0.

The m₃ is preferably an integer of 1 to 3, and more preferably 1 or 2.

The m 4 is preferably an integer of 0 to 3, and more preferably an integer of 0 to 2.

As the structural unit (II), structural units represented by the following formulas (2a-1) to (2a-10) (hereinafter also referred to as “structural units (2a-1) to (2a-10)”) and the like are preferable.

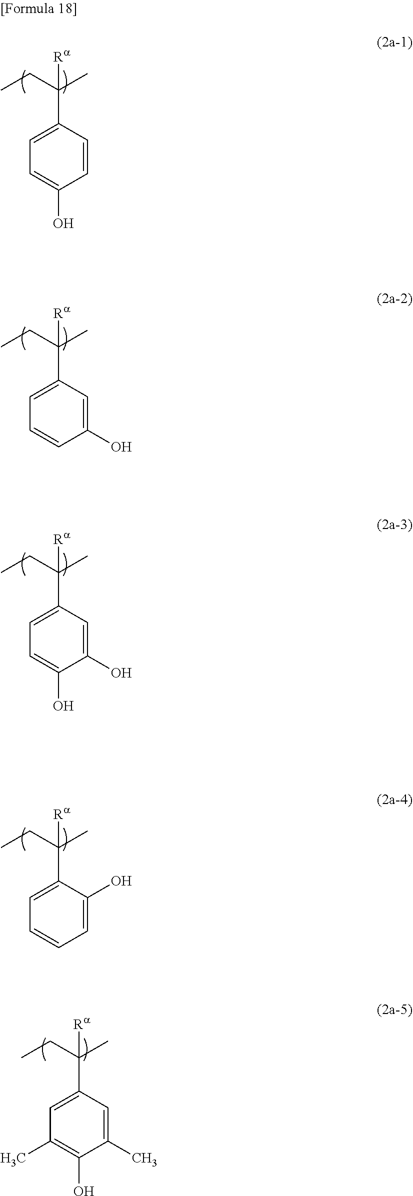

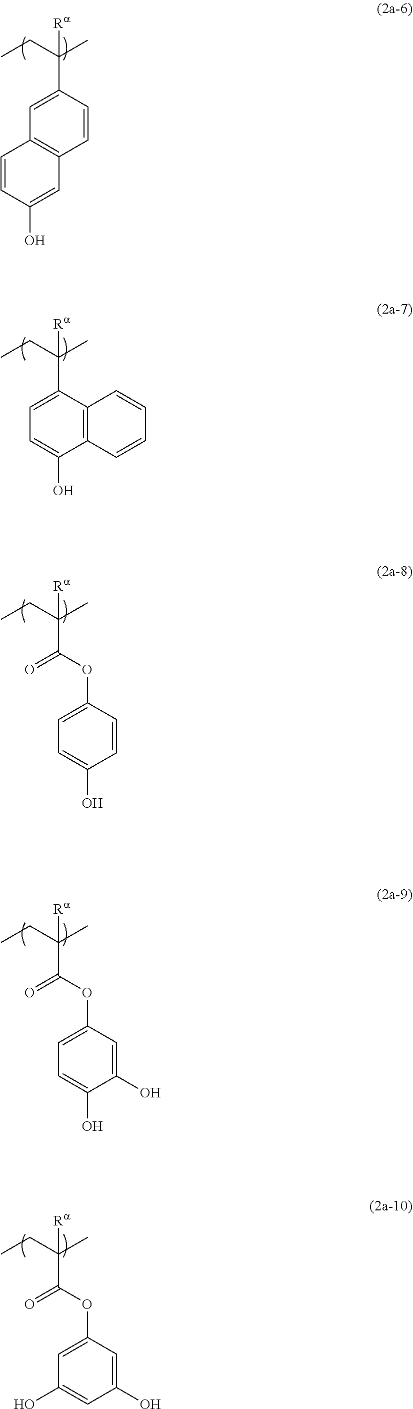

In the formulas (2a-1) to (2a-10), R^{α} is the same as in the above formula (2).

Among these, the structural units (2a-1) to (2a-4), (2a-6), (2a-8) and (2a-9) are preferable.

The content of the structural unit (II) (when a plurality of types of structural unit (II) are contained, the total content thereof is taken) is preferably 5 mol % or more, more preferably 8 mol % or more, still more preferably 10 mol % or more, and particularly preferably 15 mol % or more based on all structural units constituting the resin. The content is preferably 50 mol % or less, more preferably 40 mol % or less, still more preferably 35 mol % or less, and particularly preferably 30 mol % or less. When the content of the structural unit (II) is adjusted to within the above range, the sensitivity and CDU performance of the radiation-sensitive resin composition can be further improved.

In the case of polymerizing a monomer having a phenolic hydroxy group such as hydroxystyrene, it is preferable to polymerize the monomer with the phenolic hydroxy group protected by a protecting group such as an alkali-dissociable group and then deprotect it by hydrolysis to obtain a structural unit (II).

(Structural unit (III))

The structural unit (III) is a structural unit containing at least one structure selected from the group consisting of a lactone structure, a cyclic carbonate structure, and a sultone structure. When the base resin further has the structural unit (III), the solubility of the base resin in a developer can be adjusted, and as a result, the lithographic performance, such as resolution, of the radiation-sensitive resin composition can be improved. In addition, the adhesion between a resist pattern formed from the base resin and a substrate can be improved.

Examples of the structural unit (III) include structural units represented by the following formulas (T-1) to (T-10).

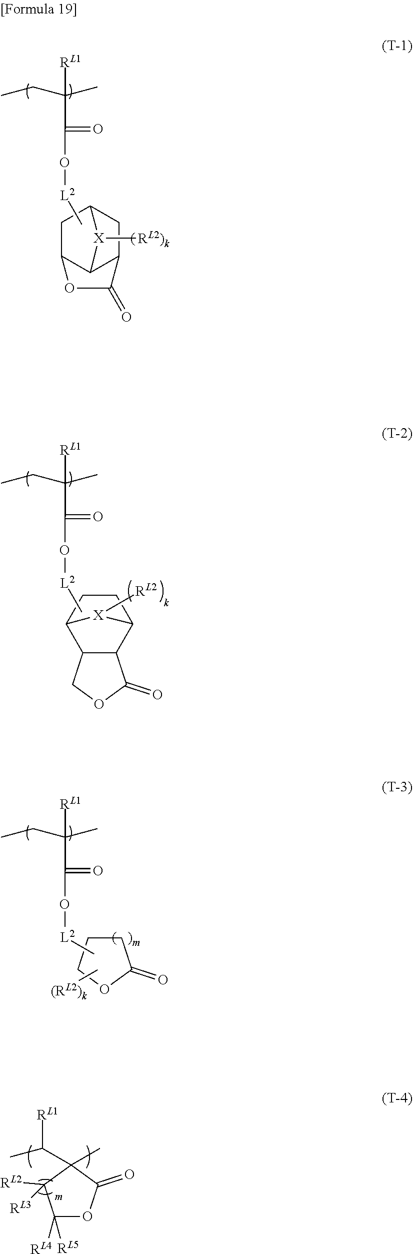

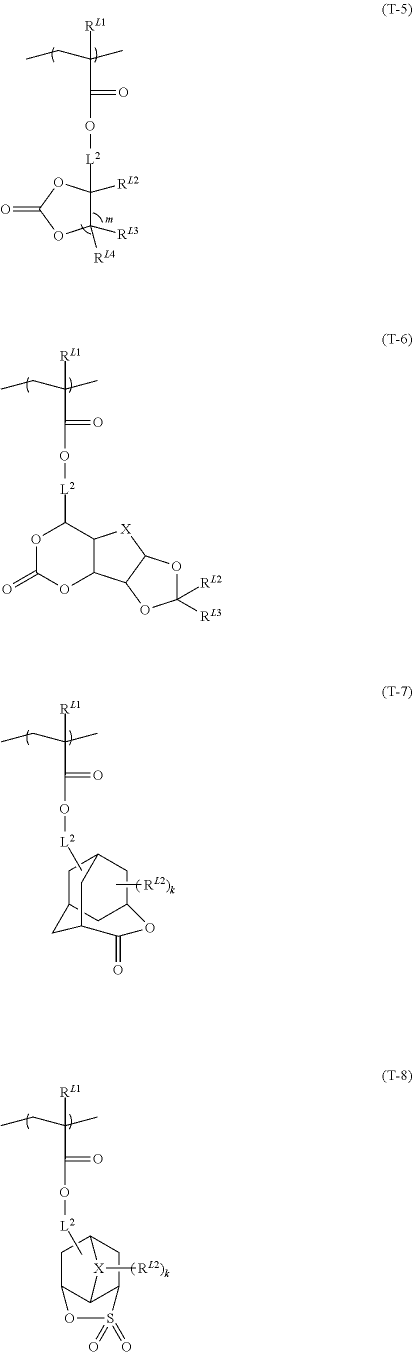

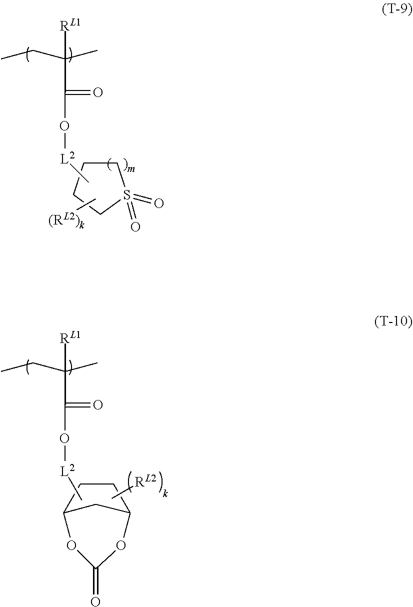

In the above formula, R^{L1} is a hydrogen atom, a fluorine atom, a methyl group, or a trifluoromethyl group. R^{L2} to R^{L5} are each independently a hydrogen atom, an alkyl group having 1 to 4 carbon atoms, a cyano group, a trifluoromethyl group, a methoxy group, a methoxycarbonyl group, a hydroxy group, a hydroxymethyl group, or a dimethylamino group. R^{L4} and R^{L5} may be combined with each other and constitute a divalent alicyclic group having 3 to 8 carbon atoms together with the carbon atom to which they are bonded. L 2 is a single bond or a divalent linking group. X is an oxygen atom or a methylene group. k is an integer of 0 to 3. m is an integer of 1 to 3.

The divalent alicyclic group having 3 to 8 carbon atoms composed of the R^{L4} and the R^{L5} combined together as well as the carbon atoms to which the R^{L4} and the R^{L5} are bonded is not particularly limited as long as it is a group formed by removing two hydrogen atoms from the same carbon atom contained in a carbon ring of a monocyclic or polycyclic alicyclic hydrocarbon having the aforementioned number of carbon atoms. The group may be either a monocyclic hydrocarbon group or a polycyclic hydrocarbon group, and the polycyclic hydrocarbon group may be either a bridged alicyclic hydrocarbon group or a fused alicyclic hydrocarbon group, and may be either a saturated hydrocarbon group or an unsaturated hydrocarbon group. It is to be noted that the fused alicyclic hydrocarbon group refers to a polycyclic alicyclic hydrocarbon group in which two or more alicyclic rings share a side (a bond between two adjacent carbon atoms).

Examples of the divalent linking group represented by L 2 include a divalent linear or branched hydrocarbon group having 1 to 10 carbon atoms, a divalent alicyclic hydrocarbon group having 4 to 12 carbon atoms, and a group composed of one or more among these hydrocarbon groups and at least one group among —CO—, —O—, —NH—, and —S.

Among them, the structural unit (III) is preferably a structural unit containing a lactone structure, more preferably a structural unit containing a norbornane lactone structure, and still more preferably a structural unit derived from norbornane lactone-yl (meth)acrylate.

The content of the structural unit (III) (when there are a plurality of types of structural unit (III), a total content thereof is taken) is preferably 5 mol % or more, more preferably 10 mol % or more, and still more preferably 15 mol % or more based on all structural units constituting the base resin. The content is preferably 50 mol % or less, more preferably 40 mol % or less, and still more preferably 35 mol % or less. When the content of the structural unit (III) is adjusted to within the range, the lithographic performance, such as resolution, of the radiation-sensitive resin composition and the adhesion between a resist patter to be formed and a substrate can be further improved.

(Other Structural Unit)

The base resin optionally has other structural units in addition to the structural units (I) to (III). Examples of the other structural unit include structural units (IV) containing a polar group, provided that those corresponding to the structural units (II) and (III) are excluded, and other structural units (V) having an acid-dissociable group, provided that those corresponding to the structural unit (I) are excluded.

(Structural Unit (IV))

When the base resin further has the structural unit (IV), the solubility of the base resin in a developer can be adjusted, and as a result, the lithographic performance, such as resolution, of the radiation-sensitive resin composition can be improved. Examples of the polar group include a hydroxy group, a carboxy group, a cyano group, a nitro group, and a sulfonamide group. Among them, a hydroxy group and a carboxy group are preferable, and a hydroxy group is more preferable.

Examples of the structural unit (IV) include structural units represented by the following formulas.

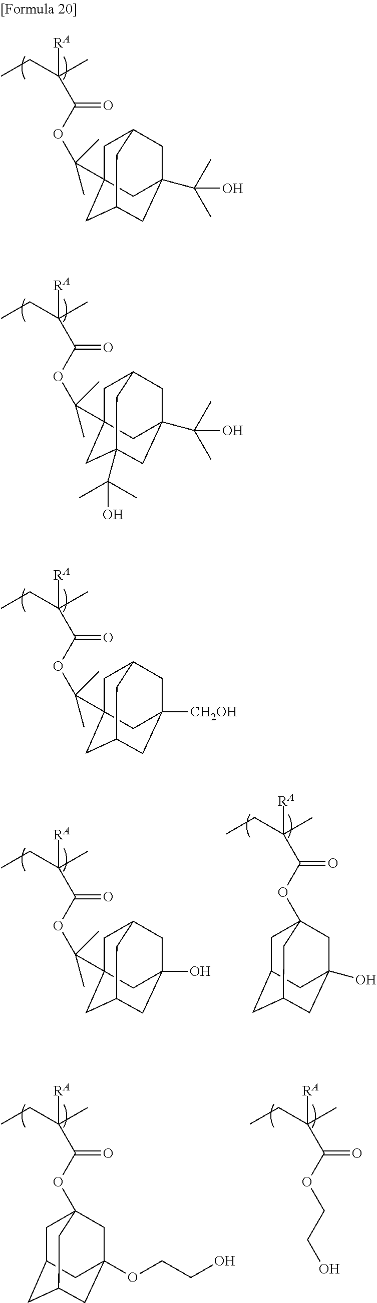

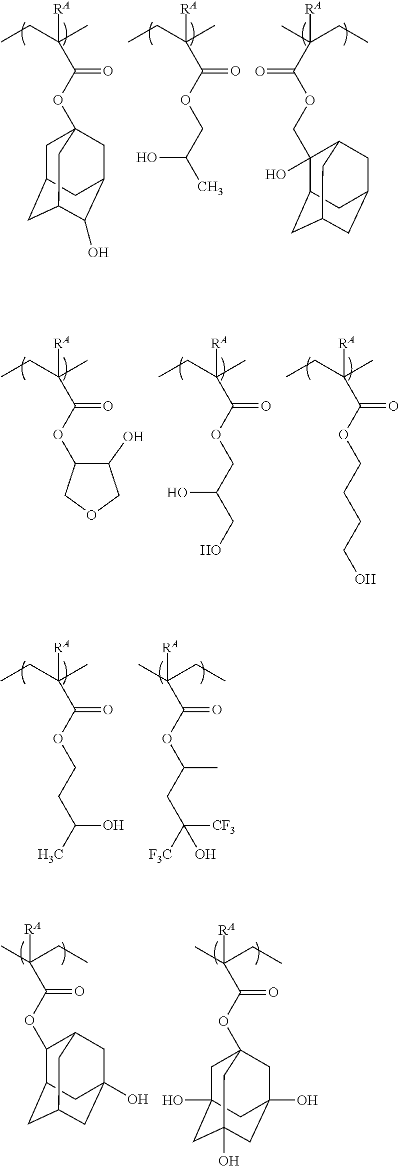

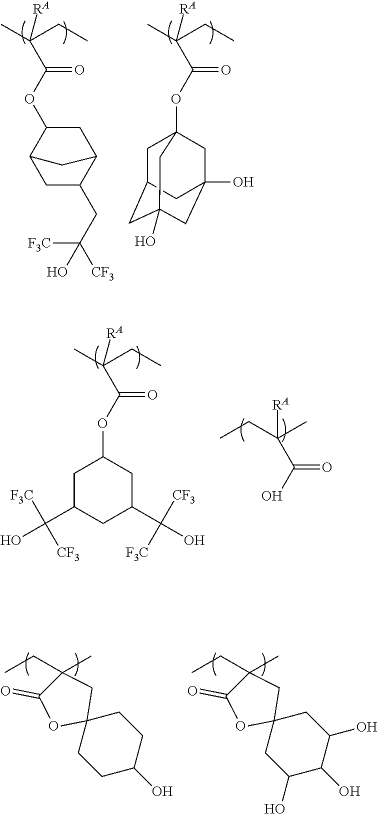

In the above formulas, RA is a hydrogen atom, a fluorine atom, a methyl group, or a trifluoromethyl group.

When the base resin has the structural unit (IV), the lower limit of the content of the structural unit (IV) (when a plurality of types of structural unit (IV) are contained, the total content thereof is taken) is preferably 1 mol %, more preferably 5 mol %, still more preferably 10 mol % based on all structural units constituting the base resin. The upper limit of the content is preferably 40 mol %, more preferably 30 mol %, and still more preferably 25 mol %. When the content of the structural unit (IV) is adjusted to within the above range, the lithographic performance, such as resolution, of the radiation-sensitive resin composition can be further improved.

(Structural Unit (V))

The structural unit (V) is a structural unit containing an acid-dissociable group, provided that the structural unit is different from the structural unit (I) and the structural unit (II). The structural unit (V) is not particularly limited as long as it contains an acid-dissociable group, and examples thereof include a structural unit having a tertiary alkyl ester moiety, a structural unit having a structure in which a hydrogen atom of a phenolic hydroxy group is substituted with a tertiary alkyl group, and a structural unit having an acetal bond. From the viewpoint of improving the patternability of the radiation-sensitive resin composition, a structural unit represented by the following formula (3) (hereinafter also referred to as “structural unit (V-1)”) is preferable.

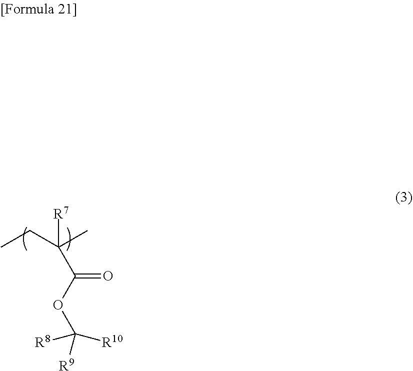

In the above formula (3), R⁷ is a hydrogen atom, a fluorine atom, a methyl group, or a trifluoromethyl group. R⁸ is a monovalent hydrocarbon group having 1 to 20 carbon atoms. R⁹ and R¹⁹ each independently represent a monovalent chain hydrocarbon group having 1 to 10 carbon atoms or a monovalent alicyclic hydrocarbon group having 3 to 20 carbon atoms, or represent a divalent alicyclic group having 3 to 20 carbon atoms in which such groups are combined with each other and which is constituted of such groups together with the carbon atoms to which such groups are bonded.

From the viewpoint of the copolymerizability of a monomer that affords the structural unit (V-1), as the R⁷, a hydrogen atom and a methyl group are preferable, and a methyl group is more preferable.

Examples of the monovalent hydrocarbon group having 1 to 20 carbon atoms represented by R⁸ include a chain hydrocarbon group having 1 to 10 carbon atoms, a monovalent alicyclic hydrocarbon group having 3 to 20 carbon atoms, and a monovalent aromatic hydrocarbon group having 6 to 20 carbon atoms.

Examples of the chain hydrocarbon groups having 1 to 10 carbon atoms represented by R⁸ to R¹⁰ include a linear or branched saturated hydrocarbon group having 1 to 10 carbon atoms and a linear or branched unsaturated hydrocarbon group having 1 to 10 carbon atoms.

Examples of the alicyclic hydrocarbon groups having 3 to 20 carbon atoms represented by R⁸ to R¹⁰ include monocyclic or polycyclic saturated hydrocarbon groups and monocyclic or polycyclic unsaturated hydrocarbon groups.

Examples of the monovalent aromatic hydrocarbon group having 6 to 20 carbon atoms represented by R⁸ include:
aryl groups such as a phenyl group, a tolyl group, a xylyl group, a naphthyl group, and an anthryl group; and aralkyl groups such as a benzyl group, a phenethyl group, and a naphthylmethyl group.
R⁸ is preferably a linear or branched saturated hydrocarbon group having 1 to 5 carbon atoms, a linear or branched unsaturated hydrocarbon group having 1 to 5 carbon atoms, an alicyclic hydrocarbon group having 3 to 12 carbon atoms, or a monovalent aromatic hydrocarbon group having 6 to 12 carbon atoms.

The divalent alicyclic group having 3 to 20 carbon atoms in which the groups represented by R⁹ and R¹⁰ are combined with each other and which is constituted of these groups together with the carbon atoms to which these groups are bonded may be either a monocyclic hydrocarbon group or a polycyclic hydrocarbon group.

Among them, it is preferable that R⁸ is an alkyl group having 1 to 4 carbon atoms and the alicyclic structure constituted by R⁹ and R¹° combined with each other and the carbon atom to which the R⁹ and R¹° are bonded is a polycyclic or monocyclic cycloalkane structure.

Examples of the structural unit (V-1) include structural units represented by the following formulas ((3-1) to (3-6) (hereinafter, also referred to as “structural units (V-1-1) to (V-1-6)”).

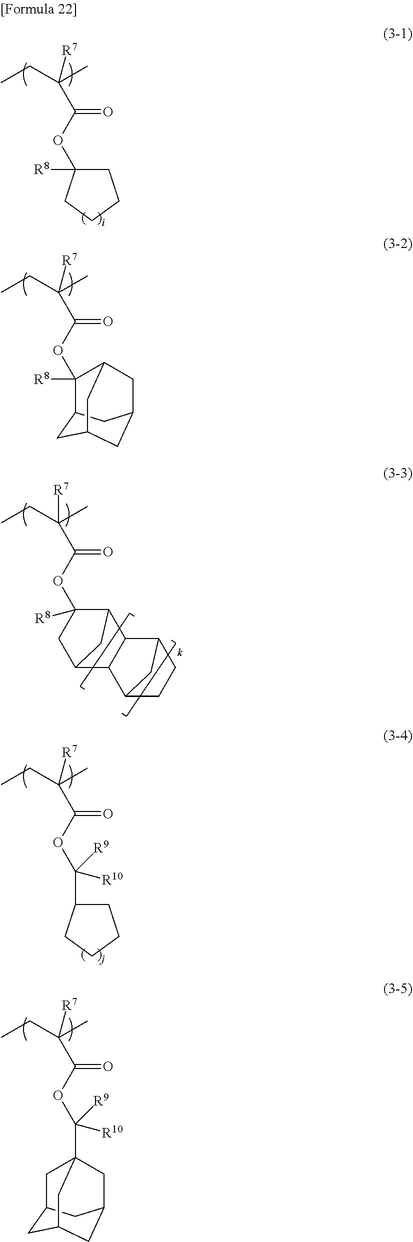

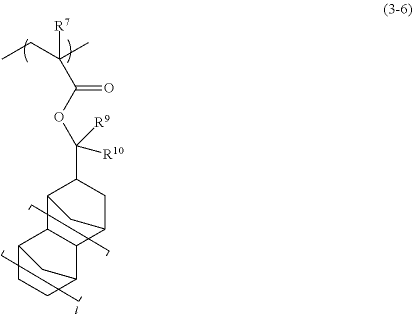

In the formulas (3-1) to (3-6), R⁷ to R¹⁰ have the same definitions as those in the above formula (3). i and j are each independently an integer of 1 to 4. k and 1 are 0 or 1.

i and j are preferably 1. R⁸ is preferably a methyl group, an ethyl group, or an isopropyl group. R⁹ and R¹⁸ are preferably a methyl group or an ethyl group.

The base resin may contain one type of the structural unit (V) or two or more types of the structural unit (V) in combination.

When the base resin contains the structural unit (V), the lower limit of the content of the structural unit (V) (the total content when a plurality of structural units (V) are contained) is preferably 3 mol %, more preferably 5 mol %, and still more preferably 10 mol % based on all structural units constituting the base resin. The upper limit of the content is preferably 50 mol %, more preferably 40 mol %, and still more preferably 30 mol %. When the content of the structural unit (V) is adjusted to within the above range, the patternability of the radiation-sensitive resin composition can be further improved.

(Method for Synthesizing Resin)

The resin as a base resin can be synthesized by, for example, subjecting monomers that will afford structural units to a polymerization reaction in an appropriate solvent using a publicly known radical polymerization initiator or the like.

The molecular weight of the resin as a base resin is not particularly limited, and the lower limit of the weight average molecular weight (Mw) as determined by Gel Permeation Chromatography (GPC) relative to standard polystyrene is preferably 1,000, more preferably 2,000, still more preferably 3,000, and particularly preferably 4,000. The upper limit of Mw is preferably 50,000, more preferably 30,000, still more preferably 15,000, and particularly preferably 12,000. When the Mw of the resin is within the above range, a resulting resist film is good in heat resistance and developability.

The ratio (Mw/Mn) of Mw to the number average molecular weight (Mn) of the resin as a base resin as determined by GPC relative to standard polystyrene is usually 1 or more and 5 or less, preferably 1 or more and 3 or less, and more preferably 1 or more and 2 or less.

The method for measuring Mw and Mn of a resin in the present description is as described in Examples.

The content of the resin is preferably 70% by mass or more, more preferably 75% by mass or more, and still more preferably 80% by mass based on the total solid content of the radiation-sensitive resin composition.

<Other Resins>

The radiation-sensitive resin composition of the present embodiment may contain a resin having a higher in mass content of fluorine atoms than the base resin as described above (hereinafter also referred as “high-fluorine-containing resin”) as other resin. When the radiation-sensitive resin composition contains the high fluorine-containing resin, the high-fluorine-containing resin can be localized in the surface layer of a resist film compared to the base resin, and as a result, the state of the surface of the resist film and the component distribution in the resist film can be controlled to a desired state.

The high-fluorine-containing resin preferably has, for example, the structural unit (I) through the structural unit (V) in the above-described base resin singly or in combination, as necessary, and have a structural unit represented by the following formula (4) (hereinafter also referred to as “structural unit (VI)”).

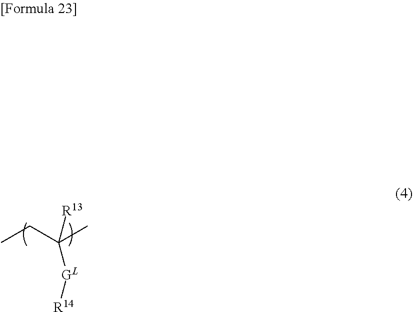

In the above formula (4), R¹³ is a hydrogen atom, a methyl group, or a trifluoromethyl group. G is a single bond, an oxygen atom, a sulfur atom, —COO—, —SO₂ONH—, —CONH—, or —OCONH—. R¹⁴ is a monovalent fluorinated chain hydrocarbon group having 1 to 20 carbon atoms or a monovalent fluorinated alicyclic hydrocarbon group having 3 to 20 carbon atoms.

When the high-fluorine-containing resin contains the structural unit (VI), the lower limit of the content of the structural unit (VI) is preferably 50 mol %, more preferably 60 mol %, still more preferably 70 mol %, and particularly preferably 80 mol % based on all structural units constituting the high-fluorine-containing resin. The upper limit of the content is preferably 100 mol %, more preferably 98 mol %, and still more preferably 95 mol %. When the content of the structural unit (VI) is adjusted to within the above range, the mass content of fluorine atoms in the high-fluorine-containing resin can more appropriately be adjusted and the localization in the surface layer of a resist film can be further promoted.

The high-fluorine-containing resin may have, in addition to the structural unit (VI), a structural unit having (x) an alkali-soluble group or (y) a group that is dissociated by the action of alkali to increase in solubility in an alkaline developer (hereinafter also referred to as structural unit (VII)). When the high-fluorine-containing resin has the structural unit (VII), solubility in an alkaline developer is improved, and the occurrence of development defects can be suppressed.

When the high-fluorine-containing resin contains the structural unit (VII), the lower limit of the content of the structural unit (VII) is preferably 10 mol %, more preferably 20 mol %, still more preferably 30 mol %, and particularly preferably 35 mol % based on all structural units constituting the high-fluorine-containing resin. The upper limit of the content is preferably 90 mol %, more preferably 75 mol %, and still more preferably 60 mol %. When the content of the structural unit (VII) is set to fall within the above range, water repellency of a resist film during immersion exposure can further be improved.

The lower limit of the content of the high-fluorine-containing resin is preferably 0.1 parts by mass, more preferably 0.5 parts by mass, still more preferably 1 part by mass, and particularly preferably 1.5 parts by mass, based on 100 parts by mass of the base resin. The upper limit of the content is preferably 12 parts by mass, more preferably 10 parts by mass, still more preferably 8 parts by mass, and particularly preferably 5 parts by mass.

(Method for Synthesizing High-Fluorine-Containing Resin)

The high-fluorine-containing resin can be synthesized by the same method as the method for synthesizing a base resin described above.

<Onium Salt>

The onium salt is a component that contains an organic acid anion moiety and an onium cation moiety and generates an acid through exposure to light. When at least part of the organic acid anion moiety in the onium salt contains an iodine-substituted aromatic ring structure, it is possible to achieve increased sensitivity due to improvement in acid generation efficiency and exhibition of CDU performance due to acid diffusion controllability.

While the mode of incorporation of the onium salt in the radiation-sensitive resin composition is not particularly limited, the onium salt is at least one member selected from the group consisting of a radiation-sensitive acid generating resin containing a structural unit having the organic acid anion moiety and the onium cation moiety, a radiation-sensitive acid generator containing the organic acid anion moiety and the onium cation moiety, and an acid diffusion controlling agent containing the organic acid anion moiety and the onium cation moiety and generating an acid having a pKa higher than that of an acid to be generated from the radiation-sensitive acid generator through irradiation with radiation. Differences between these functions will be described below.

The acid generated through the exposure to the onium salt is considered to have two functions in the radiation-sensitive resin composition depending on the strength of the acid. Examples of the first function include a function that causes the acid generated through the exposure to dissociate an acid-dissociable group of a structural unit when the resin contains the structural unit having the acid-dissociable group, to generate a carboxy group or the like. An onium salt having the first function is referred to as a radiation-sensitive acid generator. Examples of the second function include a function that controls, by salt exchange, the diffusion of the acid generated from the radiation-sensitive acid generator in the unexposed area without substantially dissociating the acid-dissociable group of the resin under a pattern formation condition using the radiation-sensitive resin composition. An onium salt having the second function is referred to as an acid diffusion controlling agent. The acid generated from the acid diffusion controlling agent can be said to be an acid relatively weaker (acid having a higher pKa) than the acid to be generated from the radiation-sensitive acid generator. Whether an onium salt functions as a radiation-sensitive acid generator or an acid diffusion controlling agent depends on the energy required for dissociating the acid-dissociable group of the resin and the acidity of the onium salt. The mode of incorporation of the radiation-sensitive acid generator in the radiation-sensitive resin composition may be a mode in which the onium salt structure is present alone as a compound (released from a polymer), a mode in which the onium salt structure is incorporated as a part of a polymer, or both of these modes. A form in which an onium salt structure is incorporated as a part of a polymer is particularly referred to as a radiation-sensitive acid generating resin.

When the radiation-sensitive resin composition contains the radiation-sensitive acid generator or a radiation-sensitive acid generating resin, the polarity of the resin in an exposed area increases, and as a result, when the developer is an aqueous alkaline solution, the resin in the exposed area is soluble in the developer, and on the other hand, when the developer is an organic solvent, the resin in the exposed area is hardly soluble in the developer.

When the radiation-sensitive resin composition contains the acid diffusion controlling agent, diffusion of an acid in an unexposed area can be controlled, and a resist pattern further superior in pattern developability and CDU performance can be formed.

In the radiation-sensitive resin composition, it is just required that the organic acid anion moiety in at least one member selected from the group consisting of the radiation-sensitive acid generating resin, the radiation-sensitive acid generator, and the acid diffusion controlling agent contains the iodine-substituted aromatic ring structure. The absorption of radiation such as EUV having a wavelength of 13.5 nm by iodine atoms is very large, so that the sensitivity is increased. In addition, when the organic acid anion moiety of the onium salt contains an iodine-substituted aromatic ring structure, acid diffusion can be controlled owing to the largeness of the molecular weight of the iodine atom and the CDU performance can be improved.

Even in any mode of incorporation of the onium salt, the organic acid anion moiety preferably has at least one type of anion selected from the group consisting of a sulfonate anion, a carboxylate anion, and a sulfonimide anion. The onium cation is preferably at least one selected from the group consisting of a sulfonium cation and an iodonium cation. When the onium salt has a combination of these structures, the above-described function can be efficiently exhibited.

Examples of the acid to be generated through the exposure include acids that generate sulfonic acid, carboxylic acid, and sulfonimide through exposure with correspondence to the organic acid anion.

Examples of an onium salt that affords a sulfonic acid through exposure include:
(1) a compound in which one or more fluorine atoms or fluorinated hydrocarbon groups are bonded to a carbon atom adjacent to a sulfonate anion, and
(2) a compound in which neither fluorine atom nor fluorinated hydrocarbon group is bonded to a carbon atom adjacent to a sulfonate anion.

Examples of an onium salt that affords a carboxylic acid through exposure include:
(3) a compound in which one or more fluorine atoms or fluorinated hydrocarbon groups are bonded to a carbon atom adjacent to a carboxylate anion, and
(4) a compound in which neither fluorine atom nor fluorinated hydrocarbon group is bonded to a carbon atom adjacent to a carboxylate anion.

Among them, as the radiation-sensitive acid generator or the radiation-sensitive acid generating resin, those corresponding to the above (1) are preferable. As the acid diffusion controlling agent, those corresponding to the above (2), (3), or (4) are preferable, and those corresponding to the above (2) or (4) are particularly preferable.

<Radiation-Sensitive Acid Generator>

The onium salt as a radiation-sensitive acid generator contains an organic acid anion moiety and an onium cation moiety. The radiation-sensitive acid generator is preferably represented by the following formula (A-1) or (A-2).

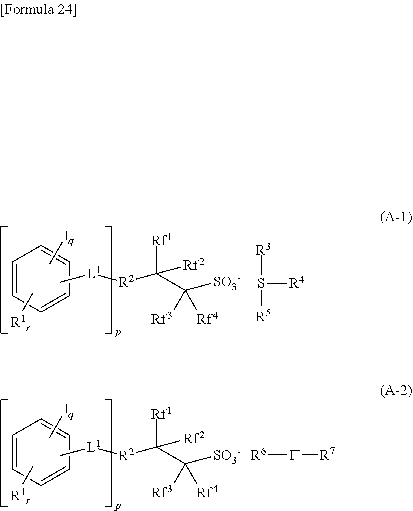

In the formulas (A-1) and (A-2), L 1 is a single bond, an ether linkage, an ester linkage, or an alkylene group having 1 to 6 carbon atoms and optionally containing an ether linkage or an ester linkage. The alkylene group may be linear, branched, or cyclic.

R¹ is a hydroxy group, a carboxy group, a fluorine atom, a chlorine atom, a bromine atom, or an amino group; or is an alkyl group having 1 to 20 carbon atoms, an alkoxy group having 1 to 20 carbon atoms, an alkoxycarbonyl group having 2 to 10 carbon atoms, an acyloxy group having 2 to 20 carbon atoms, or an alkylsulfonyloxy group having 1 to 20 carbon atoms, each optionally containing a fluorine atom, a chlorine atom, a bromine atom, a hydroxy group, an amino group, or an alkoxy group having 1 to 10 carbon atoms; or is —NR⁸—C(═O)—R⁹ or —NR⁸—C(═O)—O—R⁹, wherein R⁸ is a hydrogen atom or an alkyl group having 1 to 6 carbon atoms and optionally containing a halogen atom, a hydroxy group, an alkoxy group having 1 to 6 carbon atoms, an acyl group having 2 to 6 carbon atoms, or an acyloxy group having 2 to 6 carbon atoms, and R⁹ is an alkyl group having 1 to 16 carbon atoms, an alkenyl group having 2 to 16 carbon atoms, or an aryl group having 6 to 12 carbon atoms and optionally contains a halogen atom, a hydroxy group, an alkoxy group having 1 to 6 carbon atoms, an acyl group having 2 to 6 carbon atoms, or an acyloxy group having 2 to 6 carbon atoms. The alkyl group, alkoxy group, alkoxycarbonyl group, acyloxy group, acyl group, and alkenyl group may be linear, branched, or cyclic.

Among them, R¹ is preferably a hydroxy group, —NR⁸—C(═O)—R⁹, a fluorine atom, a chlorine atom, a bromine atom, a methyl group, a methoxy group, or the like.

R² is a single bond or a divalent linking group having 1 to 20 carbon atoms when p is 1, and is a trivalent or tetravalent linking group having 1 to 20 carbon atoms when p is 2 or 3, and the linking groups may contain an oxygen atom, a sulfur atom, or a nitrogen atom.

Rf¹ to Rf⁴ are each independently a hydrogen atom, a fluorine atom, or a trifluoromethyl group, and at least one of Rf¹ to Rf⁴ is a fluorine atom or a trifluoromethyl group. Rf¹ and Rf² may be combined to form a carbonyl group. In particular, both Rf³ and Rf⁴ are preferably fluorine atoms.

R³, R⁴, R⁵, R⁶, and R⁷ are each independently a monovalent hydrocarbon group having 1 to 20 carbon atoms and optionally containing a hetero atom. R³, R⁴ and R⁵ contain one or more fluorine atoms, and R⁶ and R⁷ contain one or more fluorine atoms. Any two of R³, R⁴, and R⁵ may be bonded to each other to form a ring together with the sulfur atom to which they are bonded. The monovalent hydrocarbon group may be linear, branched, or cyclic, and examples thereof include an alkyl group having 1 to 12 carbon atoms, an alkenyl group having 2 to 12 carbon atoms, an alkynyl group having 2 to 12 carbon atoms, an aryl group having 6 to 20 carbon atoms, and an aralkyl group having 7 to 12 carbon atoms. Some or all of the hydrogen atoms of these groups may be replaced by a hydroxy group, a carboxy group, a halogen atom, a cyano group, an amide group, a nitro group, a mercapto group, a sultone group, a sulfone group, or a sulfonium salt-containing group, and some of the carbon atoms of these groups may be replaced by an ether linkage, an ester linkage, a carbonyl group, a carbonate group, or a sulfonic acid ester linkage.

p is an integer satisfying 1≤p≤3. q and r are integers satisfying 0≤q≤5, 0≤r≤3, and 0≤q+r≤5. q is preferably an integer satisfying 1≤q≤3, and more preferably 2 or 3. r is preferably an integer satisfying 0≤r≤2.

Examples of the organic acid anion moiety of the radiation-sensitive acid generators represented by the formulas (A-1) and (A-2) include, but are not limited to, those shown below. While all of those shown below are organic acid anion moieties having an iodine-substituted aromatic ring structure, organic acid anion moieties having no iodine-substituted aromatic ring structure that can be suitably employed include structures in which the iodine atoms in the formulas shown below are replaced by an atom or group other than an iodine atom such as a hydrogen atom or other substituent.

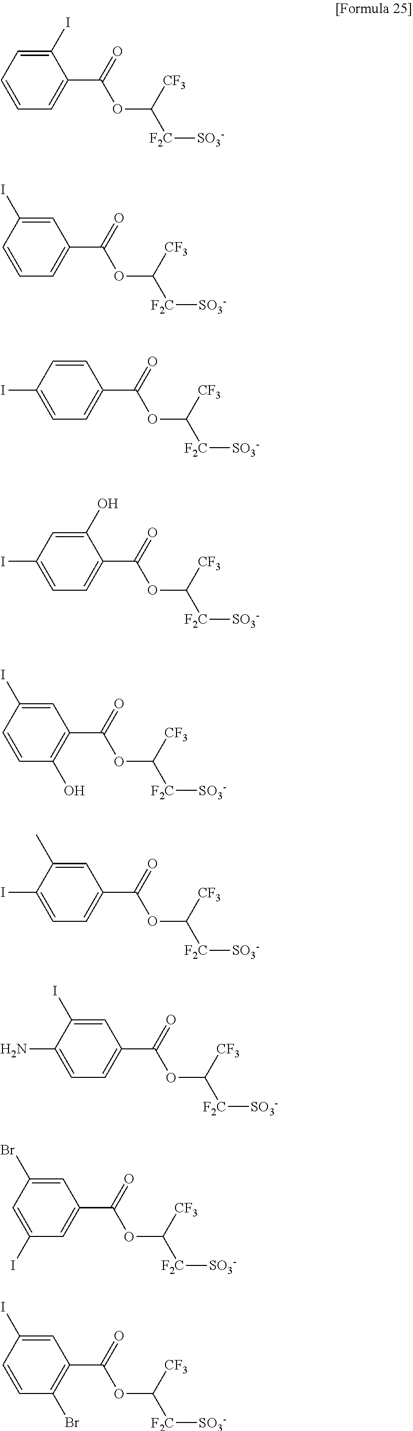

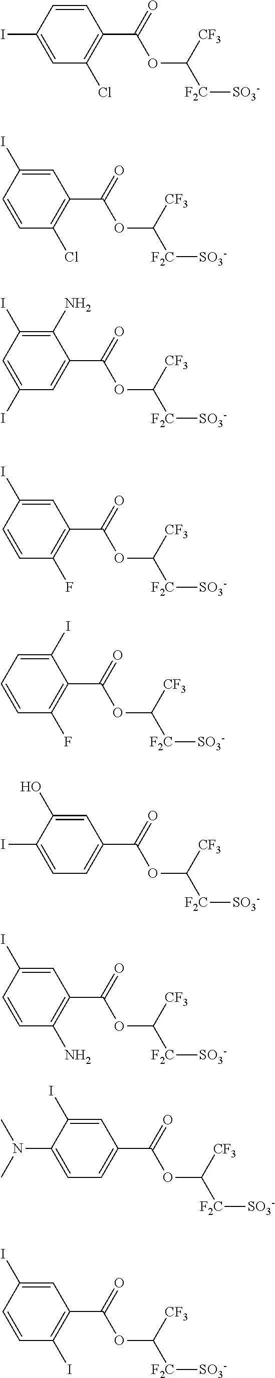

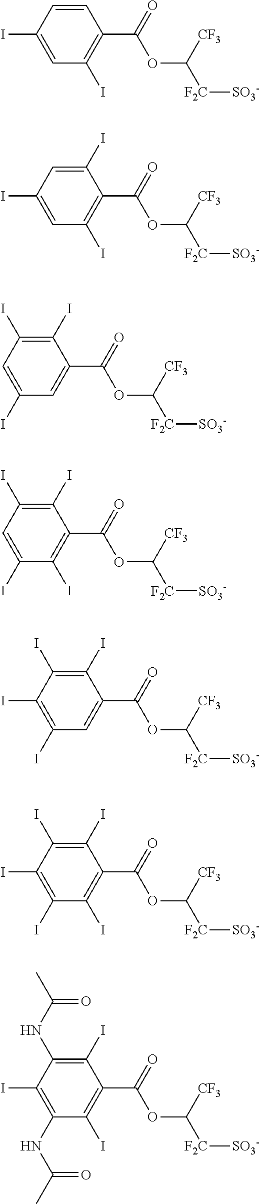

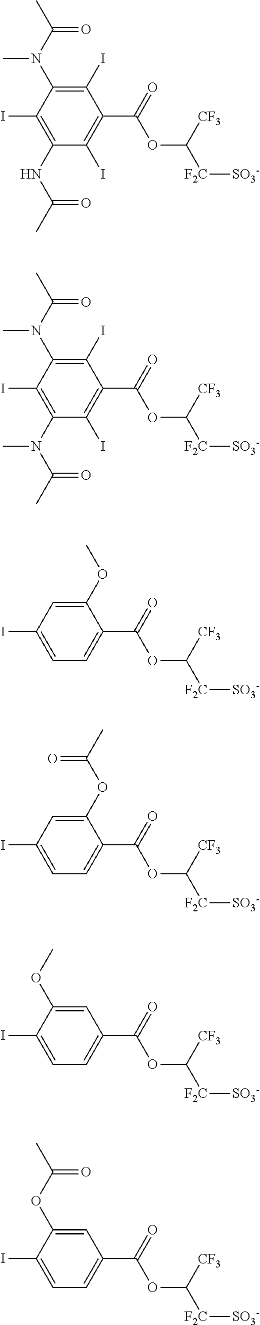

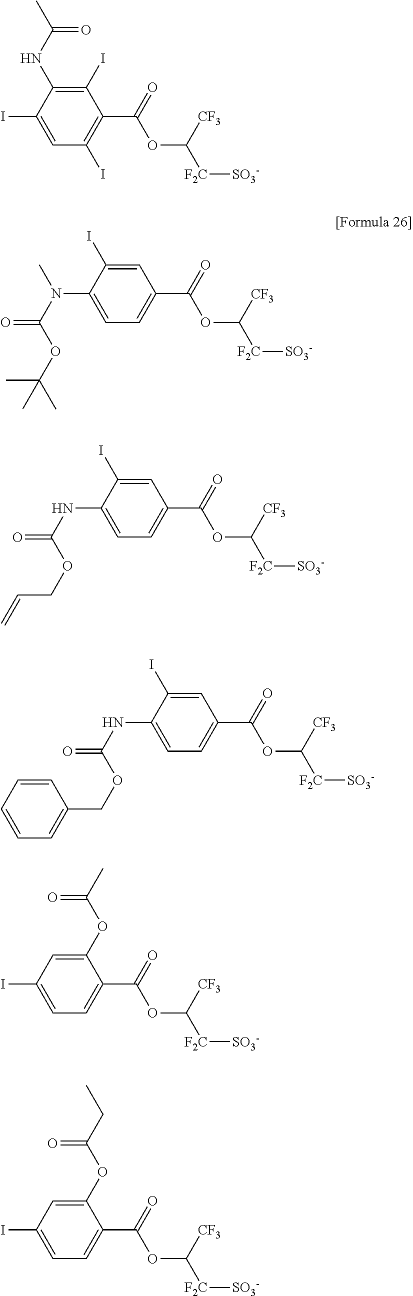

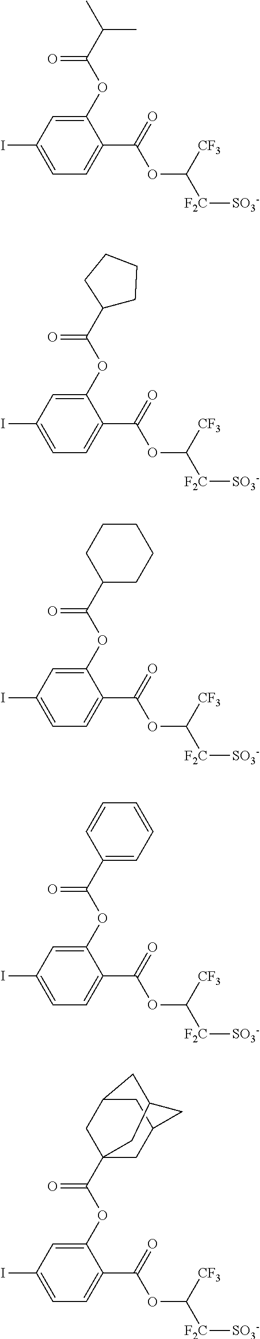

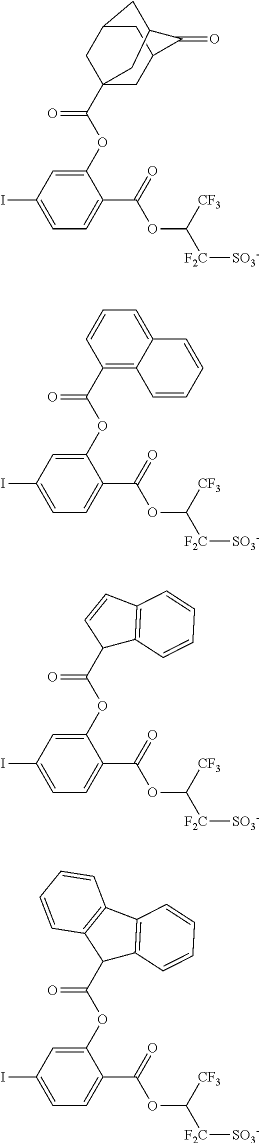

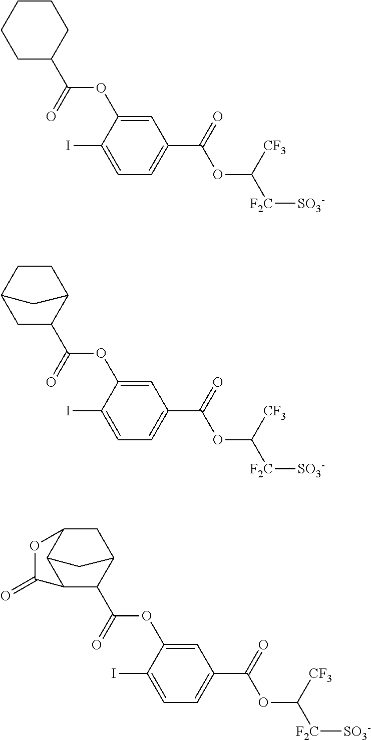

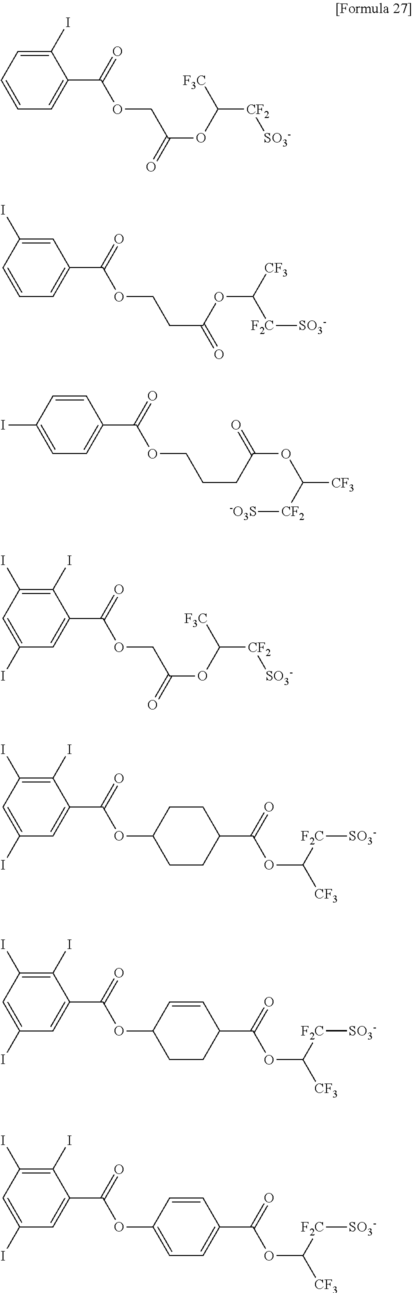

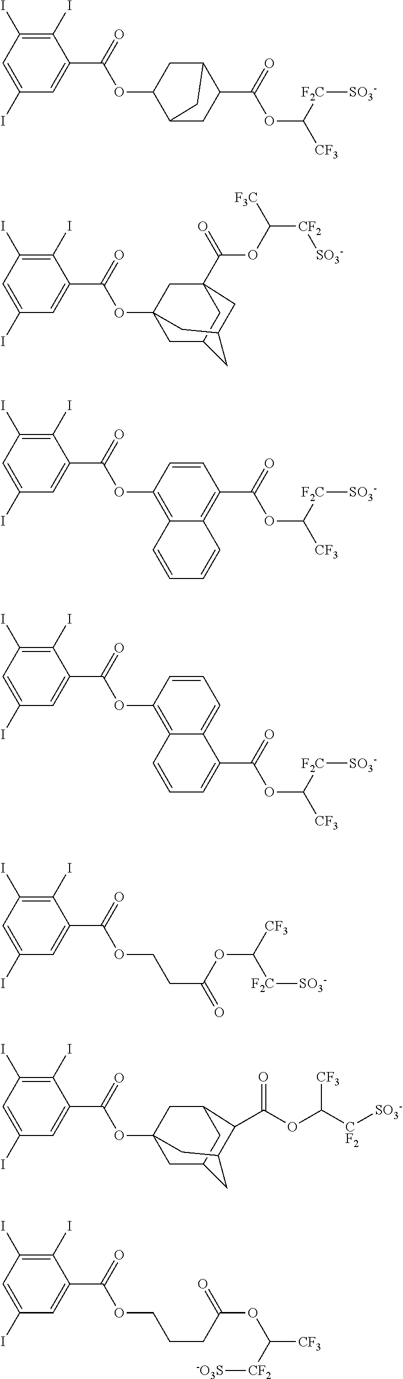

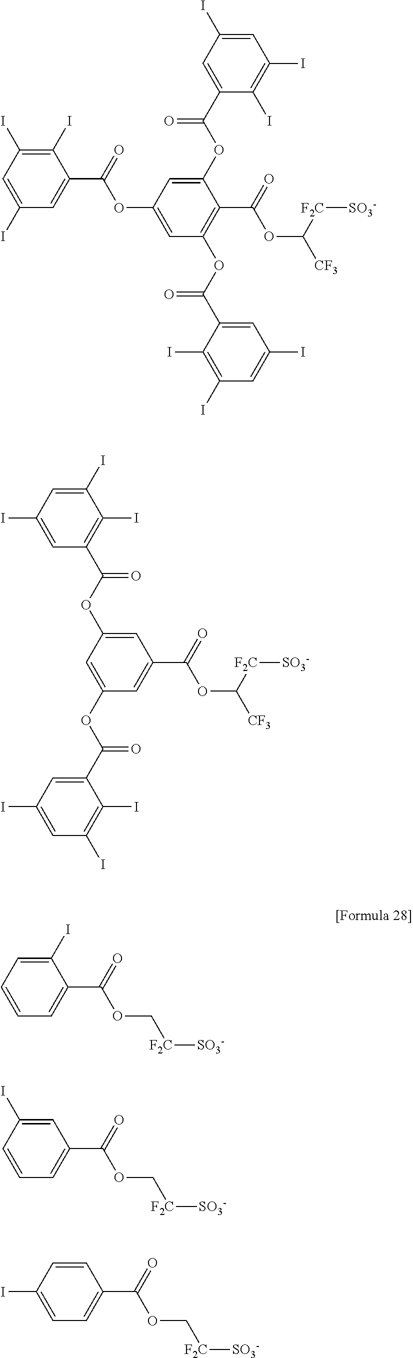

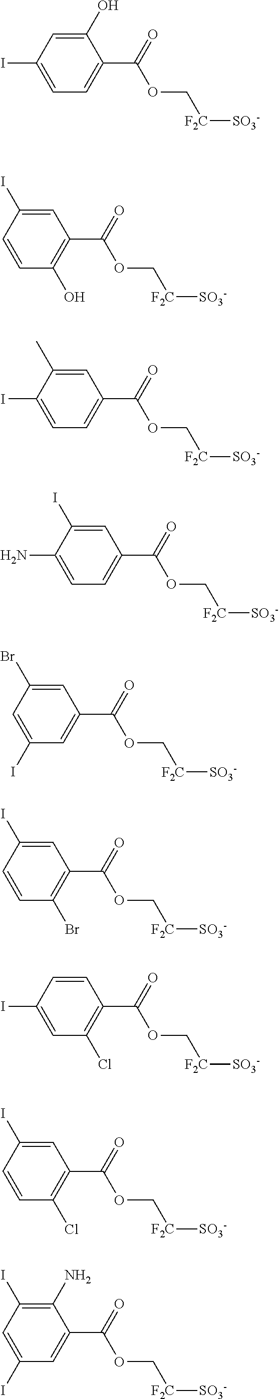

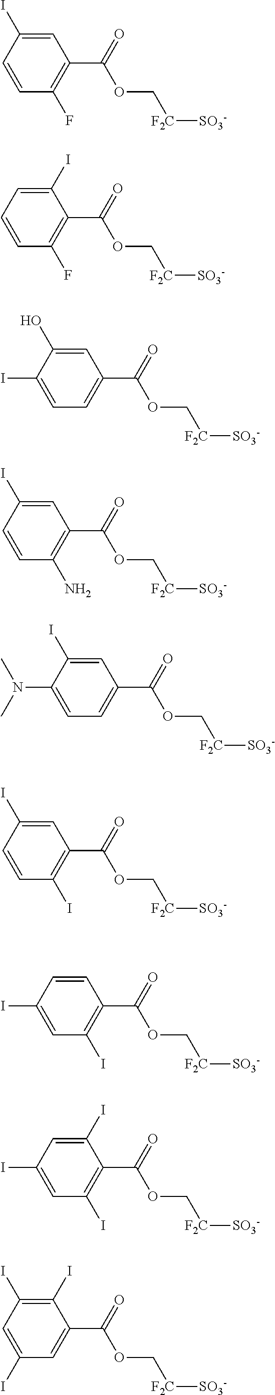

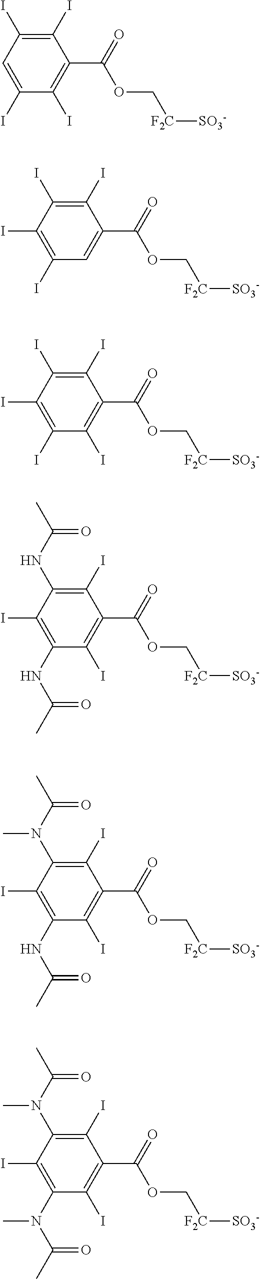

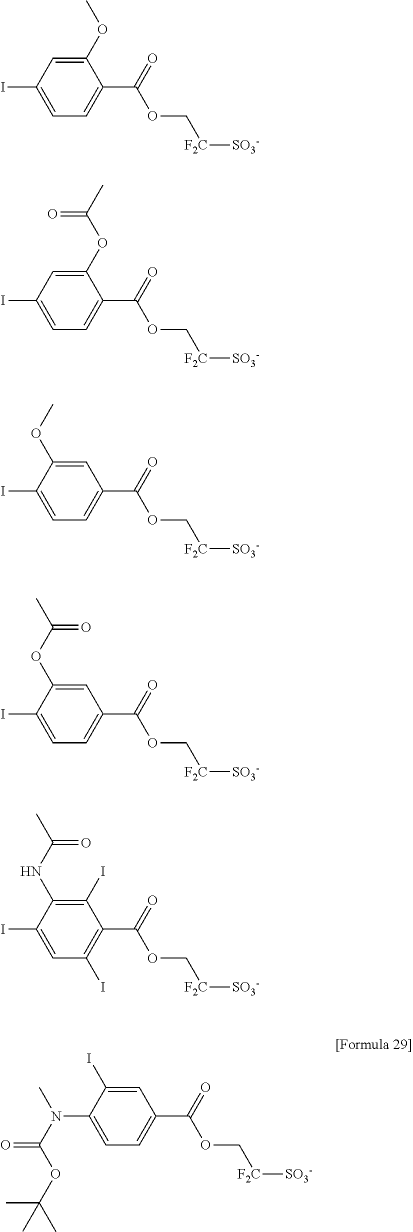

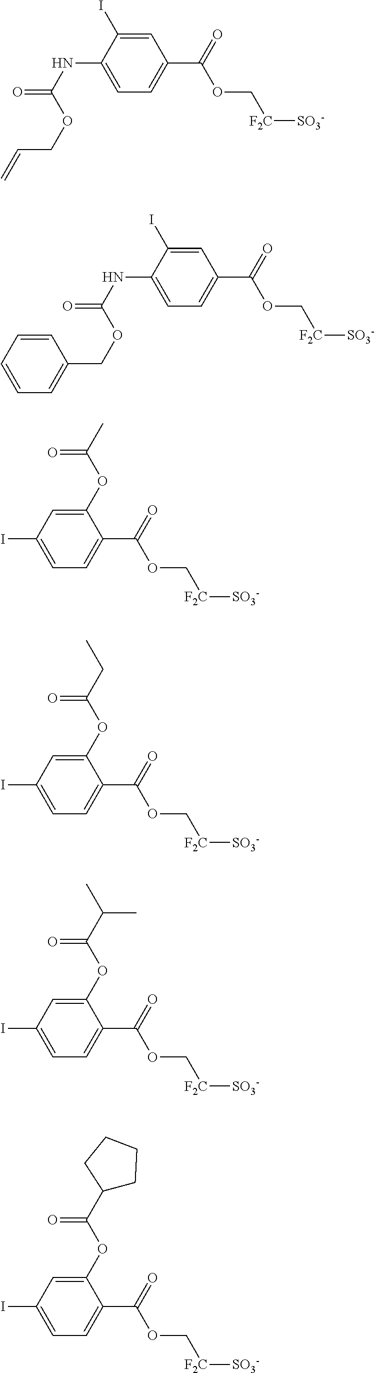

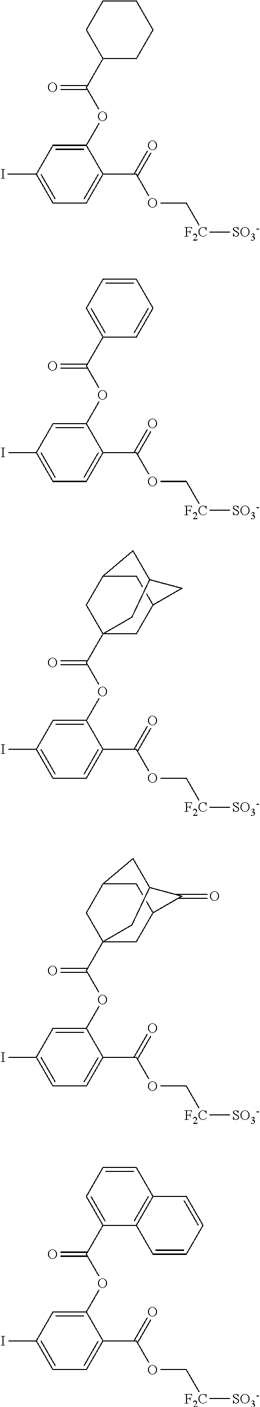

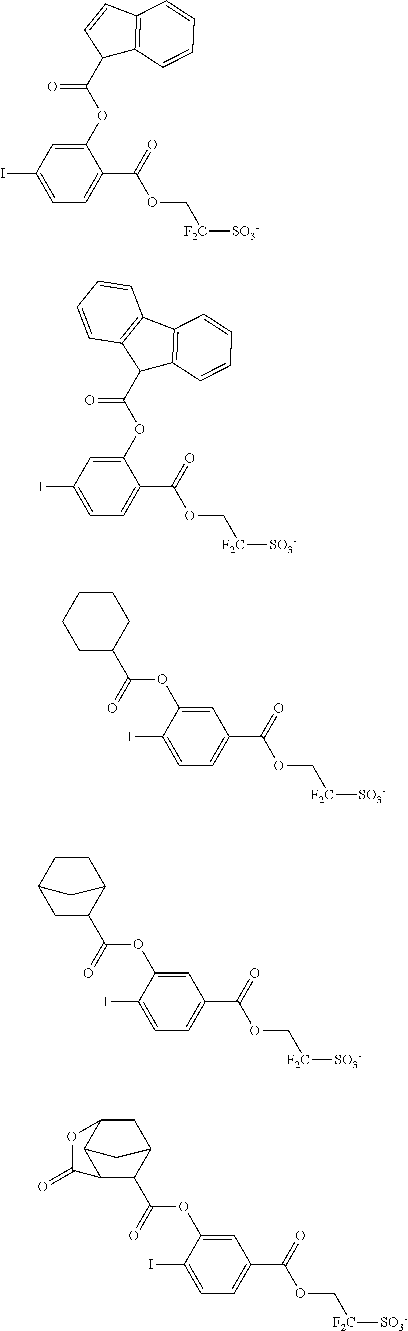

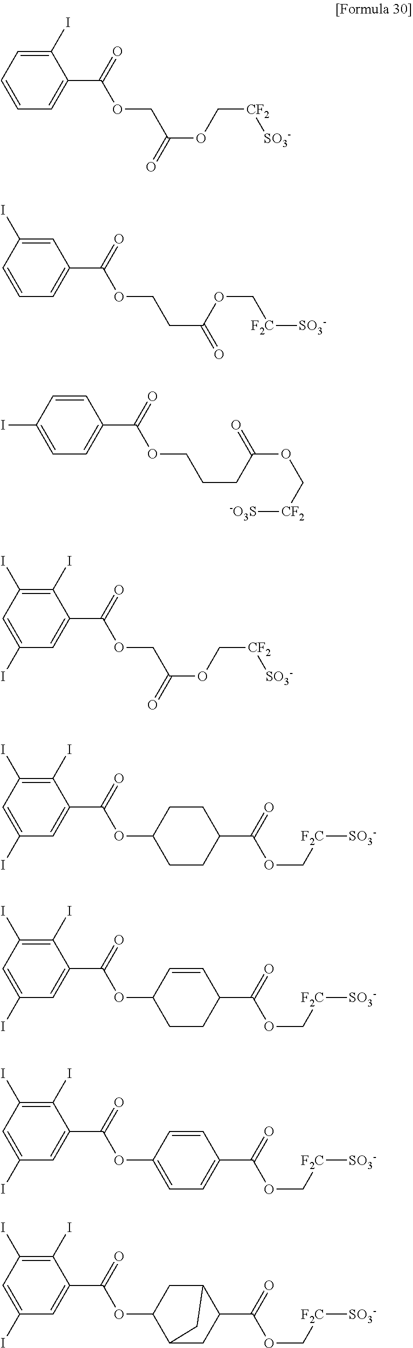

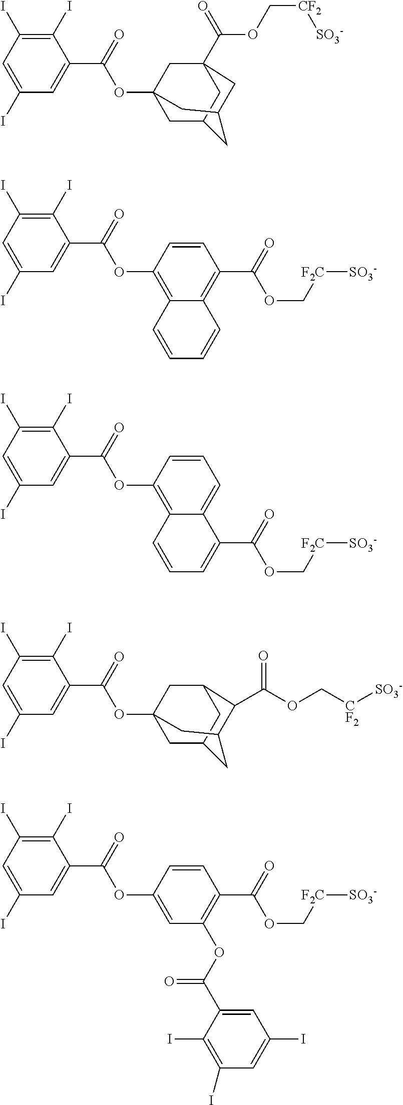

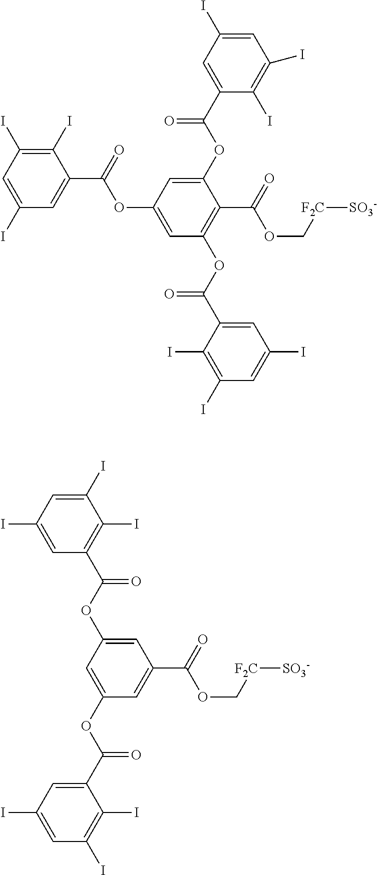

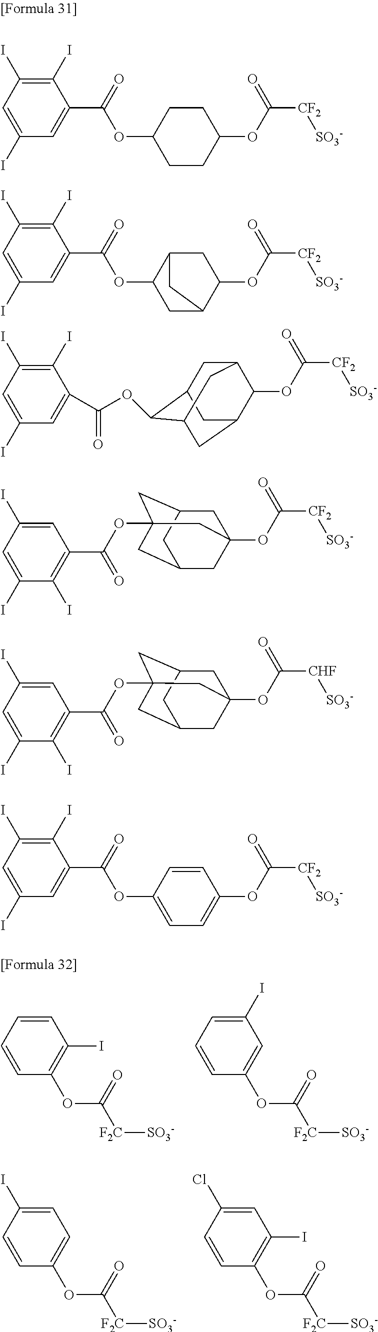

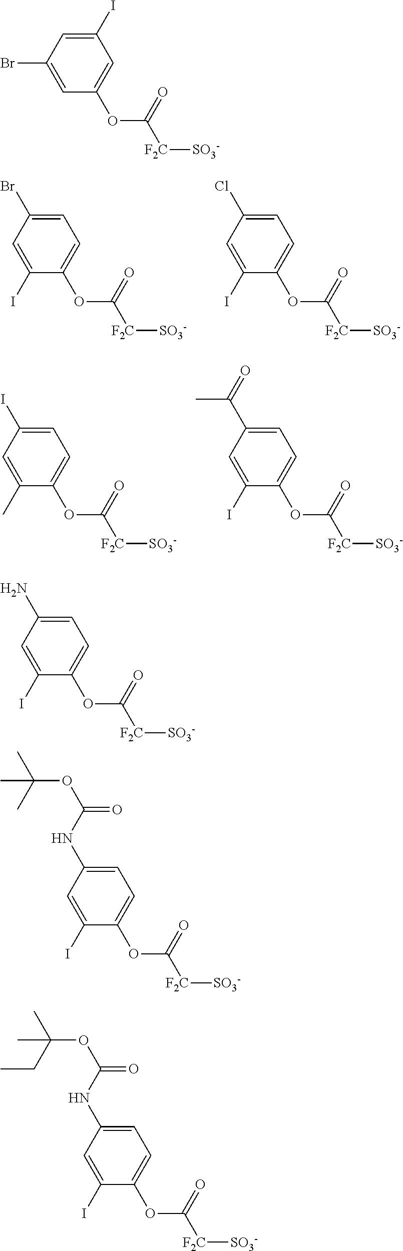

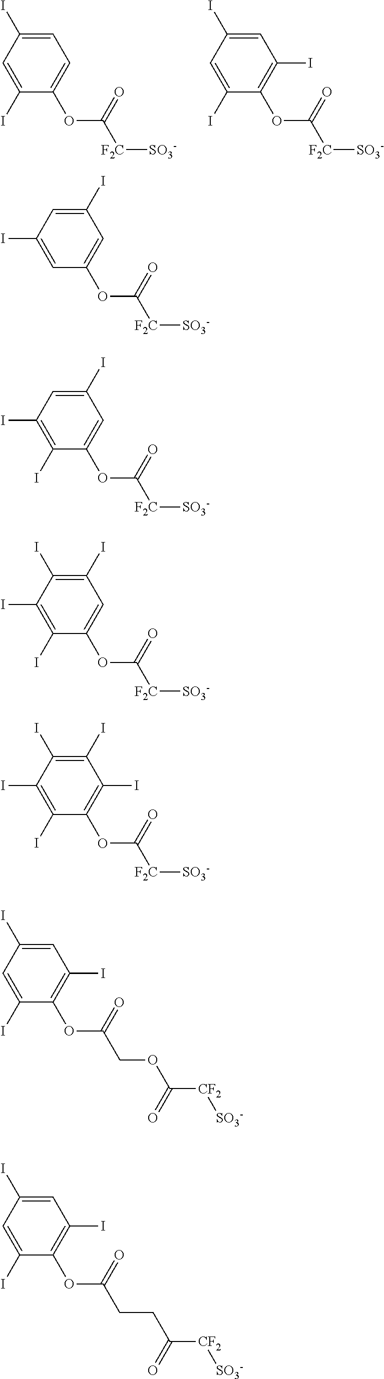

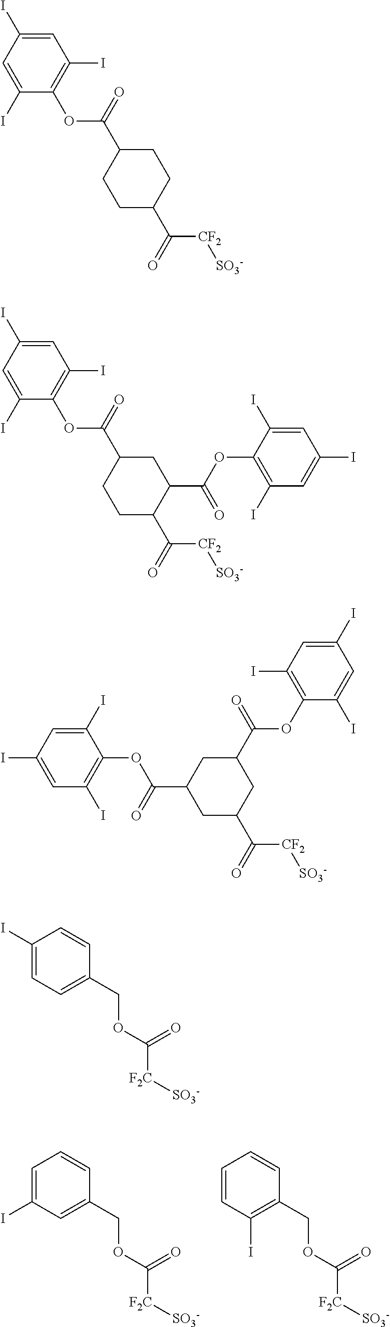

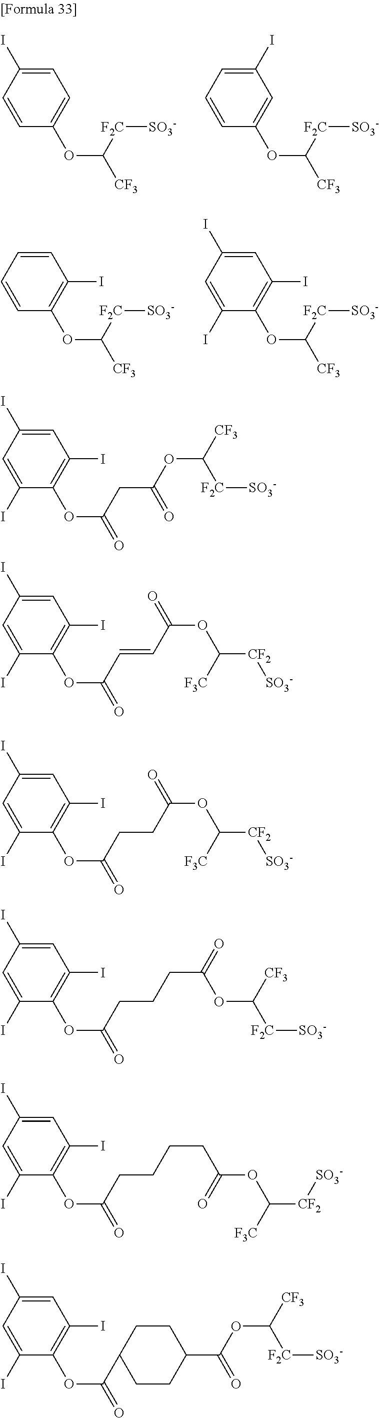

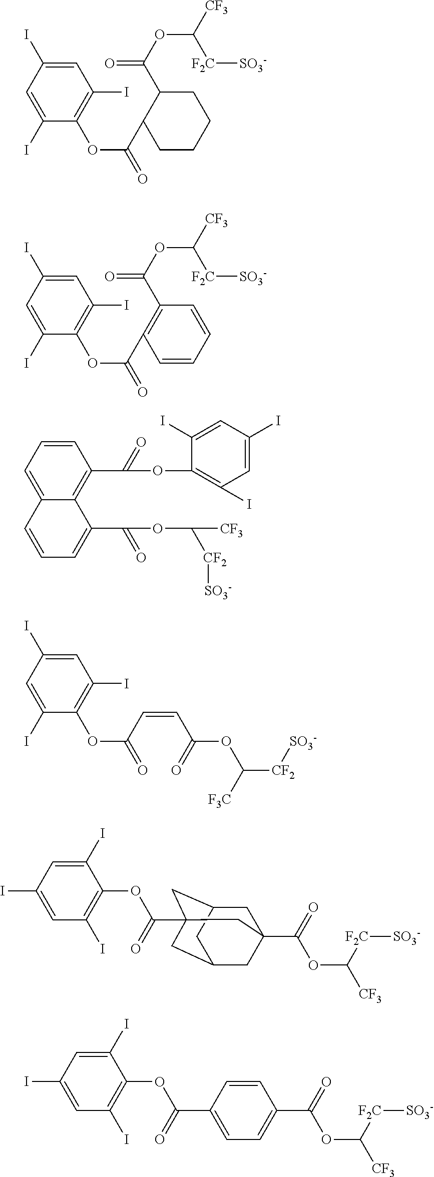

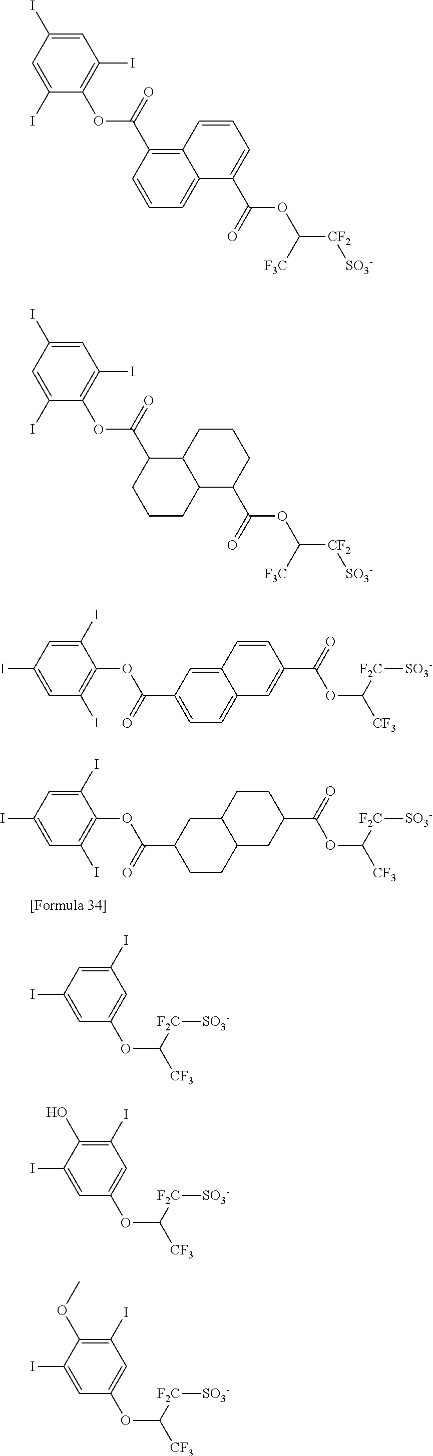

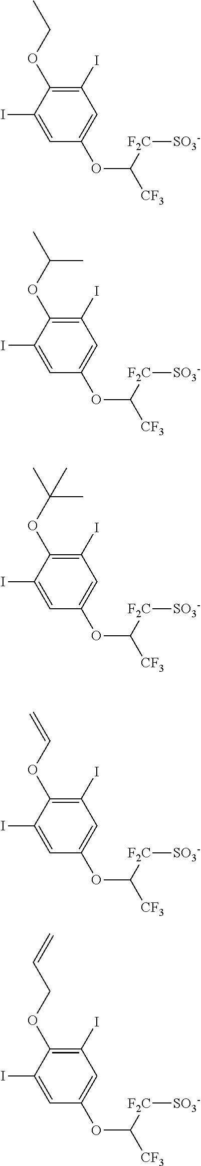

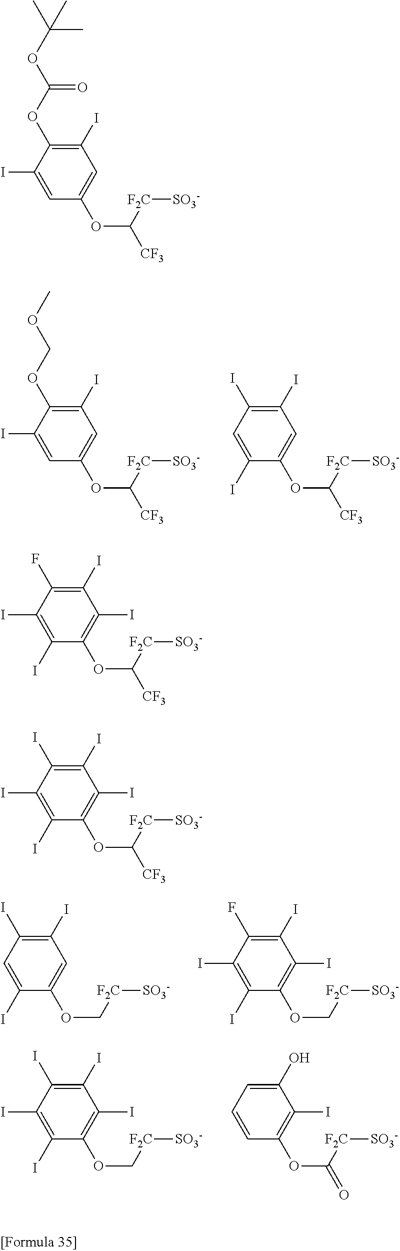

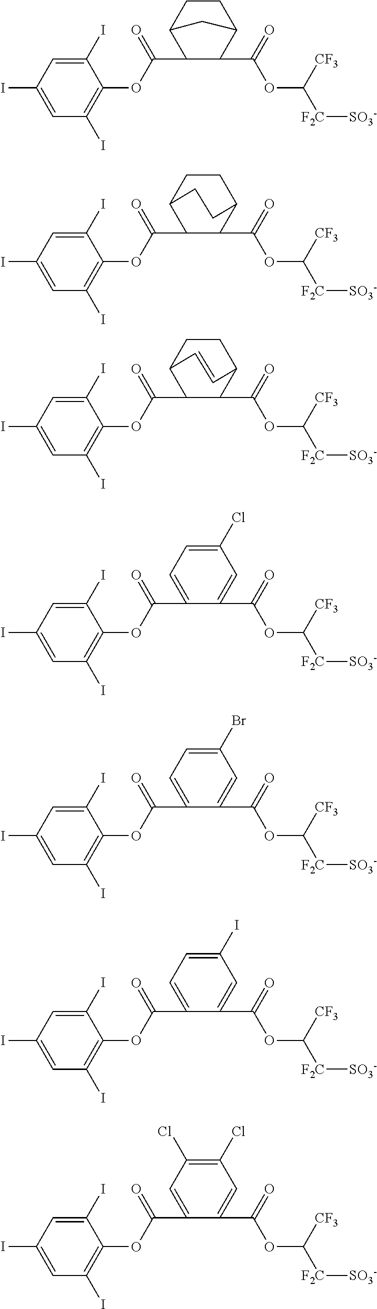

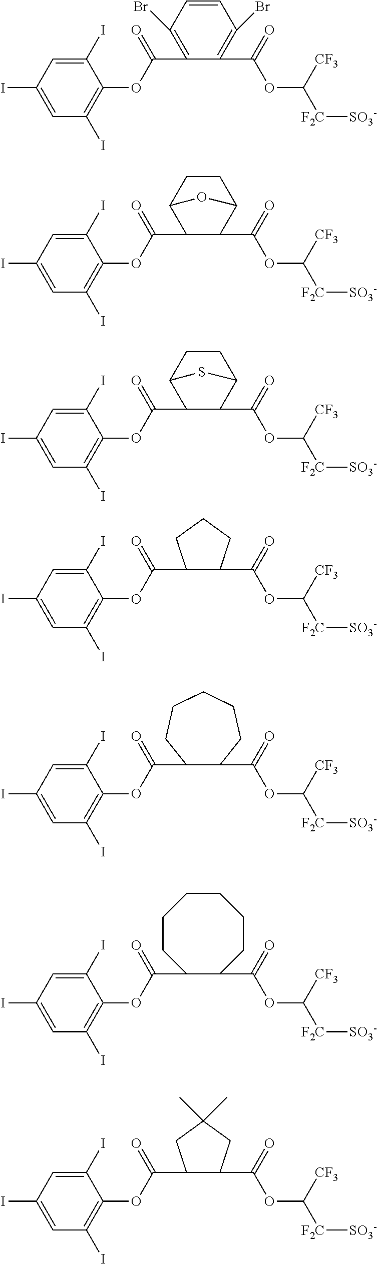

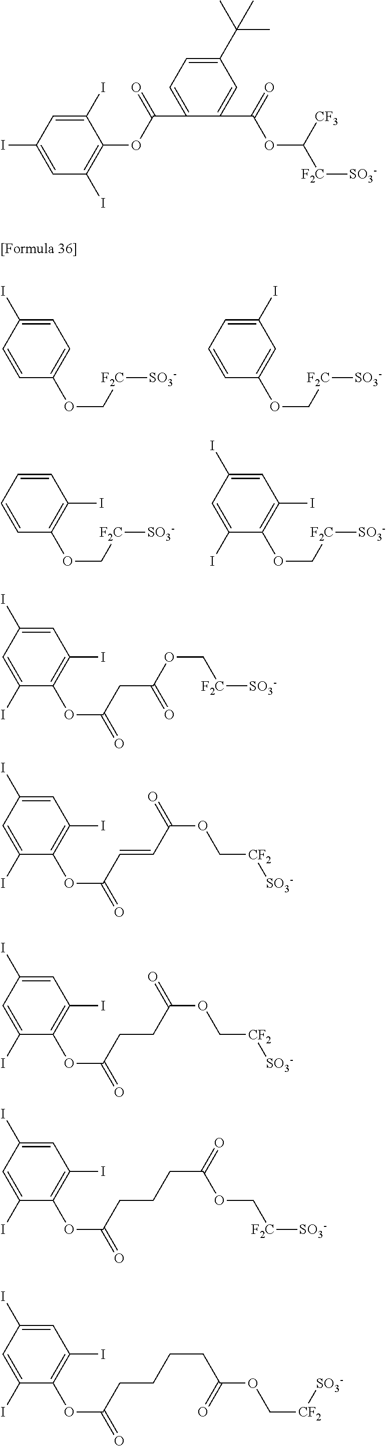

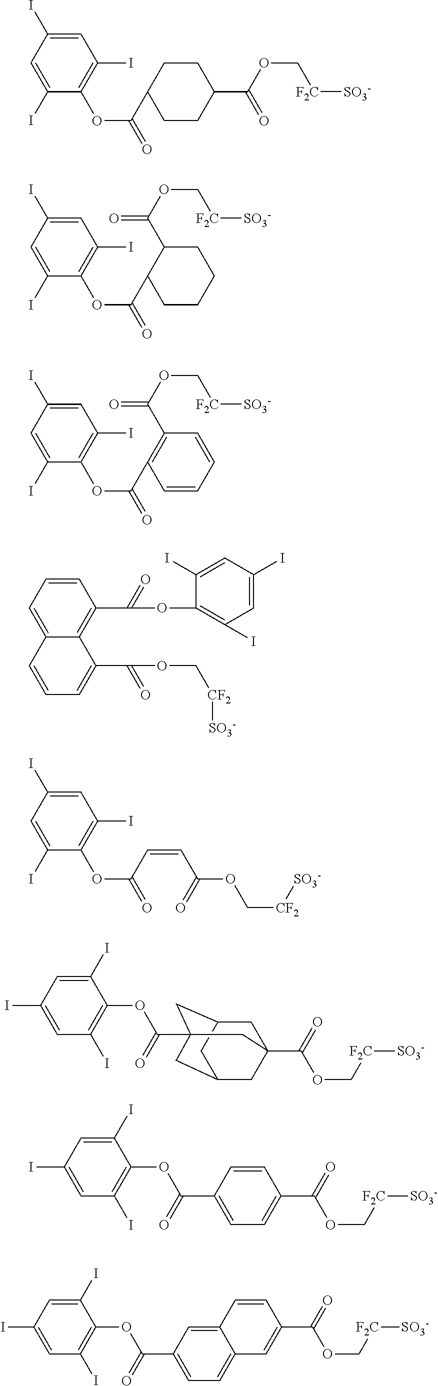

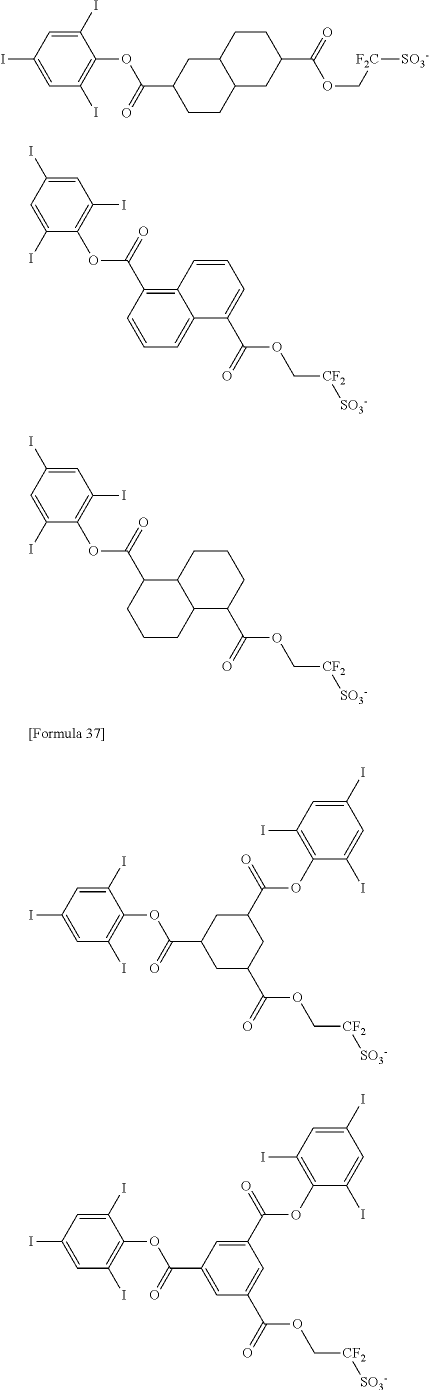

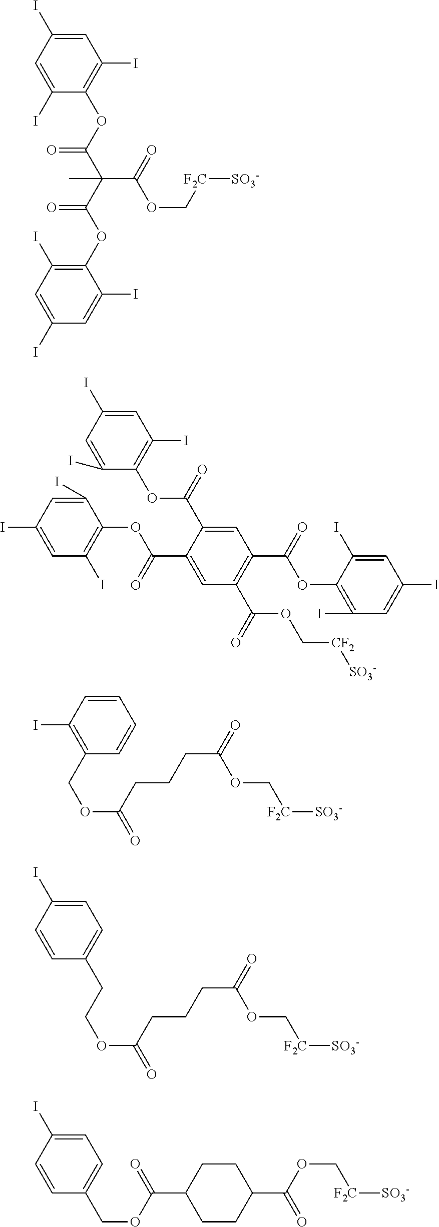

The onium cation moiety in the radiation-sensitive acid generator represented by the formula (A-1) is preferably represented by the following formula (Q-1).

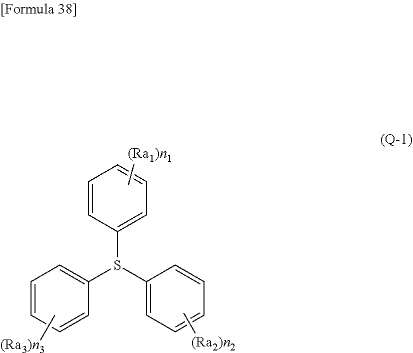

In the formula (Q-1), Ra₁ and Ra₂ each independently represent a substituent. n₁ represents an integer of 0 to 5, and when n₁ is 2 or more, the plurality of Ra₁'s may be either the same or different. n₂ represents an integer of 0 to 5, and when n₂ is 2 or more, the plurality of Ra₂'s may be either the same or different. n₃ represents an integer of 0 to 5, and when n₃ is 2 or more, the plurality of Ra₃'s may be either the same or different. Ra₃ represents a fluorine atom or a group having one or more fluorine atoms. Ra₁ and Ra₂ may be linked to each other to form a ring. When n₁ is 2 or more, the plurality of Ra₁'s may be linked to each other to form a ring. When n₂ is 2 or more, the plurality of Ra₂'s may be linked to each other to form a ring.

The substituent represented by Ra₁ and Ra₂ is preferably an alkyl group, a cycloalkyl group, an alkoxy group, a cycloalkyloxy group, an alkoxycarbonyl group, an alkylsulfonyl group, a hydroxy group, a halogen atom, or a halogenated hydrocarbon group.

The alkyl group as Ra₁ and Ra₂ may be either a linear alkyl group or a branched alkyl group. The alkyl group is preferably one having 1 to 10 carbon atoms, and particularly preferably a methyl group, an ethyl group, a n-butyl group, or a t-butyl group.

Examples of the cycloalkyl group as Ra₁ and Ra₂ include a monocyclic or polycyclic cycloalkyl group (preferably a cycloalkyl group having 3 to 20 carbon atoms). Among these, a cyclopropyl group, a cyclopentyl group, a cyclohexyl group, a cyclohexyl group, a cycloheptyl group, and a cyclooctyl group are particularly preferable.

Examples of the alkyl group moiety of the alkoxy group as Ra₁ and Ra₂ include those listed above as the alkyl group as Ra₁ and Ra₂. As the alkoxy group, a methoxy group, an ethoxy group, a n-propoxy group, and a n-butoxy group are particularly preferable.

Examples of the cycloalkyl group moiety of the cycloalkyloxy group as Ra₁ and Ra₂ include those listed above as the cycloalkyl group as Ra₁ and Ra₂. As the cycloalkyloxy group, a cyclopentyloxy group and a cyclohexyloxy group are particularly preferable.

Examples of the alkoxy group moiety of the alkoxycarbonyl group as Ra₁ and Ra₂ include those listed above as the alkoxy group as Ra₁ and Ra₂. As the alkoxycarbonyl group, a methoxycarbonyl group, an ethoxycarbonyl group, and a n-butoxycarbonyl group are particularly preferable.

Examples of the alkyl group moiety of the alkylsulfonyl group as Ra₁ and Ra₂ include those listed above as the alkyl group as Ra₁ and Ra₂. Examples of the cycloalkyl group moiety of the cycloalkylsulfonyl group as Ra₁ and Ra₂ include those listed above as the cycloalkyl group as Ra₁ and Ra₂. As the alkylsulfonyl group or the cycloalkylsulfonyl group, a methanesulfonyl group, an ethanesulfonyl group, a n-propanesulfonyl group, a n-butanesulfonyl group, a cyclopentanesulfonyl group, and a cyclohexanesulfonyl group are particularly preferable.

Each of the groups Ra₁ and Ra₂ may further have a substituent. Examples of the substituent include a halogen atom such as a fluorine atom (preferably a fluorine atom), a hydroxy group, a carboxy group, a cyano group, a nitro group, an alkoxy group, a cycloalkyloxy group, an alkoxyalkyl group, a cycloalkyloxyalkyl group, an alkoxycarbonyl group, a cycloalkyloxycarbonyl group, an alkoxycarbonyloxy group, and a cycloalkyloxycarbonyloxy group.

Examples of the halogen atom as Ra₁ and Ra₂ include a fluorine atom, a chlorine atom, a bromine atom, and an iodine atom, and a fluorine atom is preferable.

As the halogenated hydrocarbon group as Ra₁ and Ra₂, a halogenated alkyl group is preferable. Examples of the alkyl group and the halogen atom constituting the halogenated alkyl group include those described above. Among them, a fluorinated alkyl group is preferable, and CF₃ is more preferable.

As described above, Ra₁ and Ra₂ may be linked to each other to form a ring (namely, a heterocyclic ring containing a sulfur atom). In this case, Ra₁ and Ra₂ preferably form a single bond or a divalent linking group, and examples of the divalent linking group include —COO—, —OCO—, —CO—, —O—, —S—, —SO—, —SO₂—, an alkylene group, a cycloalkylene group, an alkenylene group, and a combination of two or more thereof, and those having a total carbon number of 20 or less are preferable. When n₁ is 2 or more, a plurality of Ra₁'s may be linked to each other to form a ring, and when n₂ is 2 or more, a plurality of Ra₂'s may be linked to each other to form a ring. Examples thereof include an embodiment in which two Ra₁'s are linked to each other to form a naphthalene ring together with a benzene ring to which they are bonded.

Ra₁ is a fluorine atom or a group having a fluorine atom. Examples of the group having a fluorine atom include groups in which an alkyl group, a cycloalkyl group, an alkoxy group, a cycloalkyloxy group, an alkoxycarbonyl group, and an alkylsulfonyl group as Ra₁ and Ra₂ are substituted with a fluorine atom. Among them, fluorinated alkyl groups are suitable, CF₃, C₂F₅, C₃F₇, C₄F₉, C₅F₁₁, C₆F₁₃, C₇F₁₅, C₈F₁₇, CH₂CF₃, CH₂CH₂CF₃, CH₂C₂F₅, CH₂CH₂C₂F₅, CH₂C₃F₇, CH₂CH₂C₃F₇, CH₂C₄F₉, and CH₂CH₂C₄F₉ are more suitable, and CF₃ is particularly suitable.

Ra₃ is preferably a fluorine atom or CF₃, and more preferably a fluorine atom.

n₁ and n₂ are each independently preferably an integer of 0 to 3, and preferably an integer of 0 to 2.

n₃ is preferably an integer of 1 to 3, and more preferably 1 or 2.

(n1+n2+n3) is preferably an integer of 1 to 15, more preferably an integer of 1 to 9, still more preferably an integer of 2 to 6, and particularly preferably an integer of 3 to 6. When (n1+n2+n3) is 1, it is preferable that n3=1 and Ra₃ is a fluorine atom or CF₃. When (n1+n2+n3) is 2, a combination in which n1=n3=1 and Ra₁ and Ra₃ are each independently a fluorine atom or CF₃ and a combination in which n3=2 and Ra₃ is a fluorine atom or CF₃ are preferable. When (n1+n2+n3) is 3, a combination in which n1=n2=n3=1 and Ra₁ to Ra₃ are each independently a fluorine atom or CF₃ is preferable.

Examples of such an onium cation moiety represented by the formula (Q-1) include those shown below. While all of those shown below are sulfonium cation moieties having an aromatic ring structure having a fluorine atom, onium cation moieties having no aromatic ring structure having a fluorine atom that can be suitably employed include structures in which the fluorine atoms or CF₃ in the formulas shown below are replaced by an atom or group other than a fluorine atom such as a hydrogen atom or other substituent.

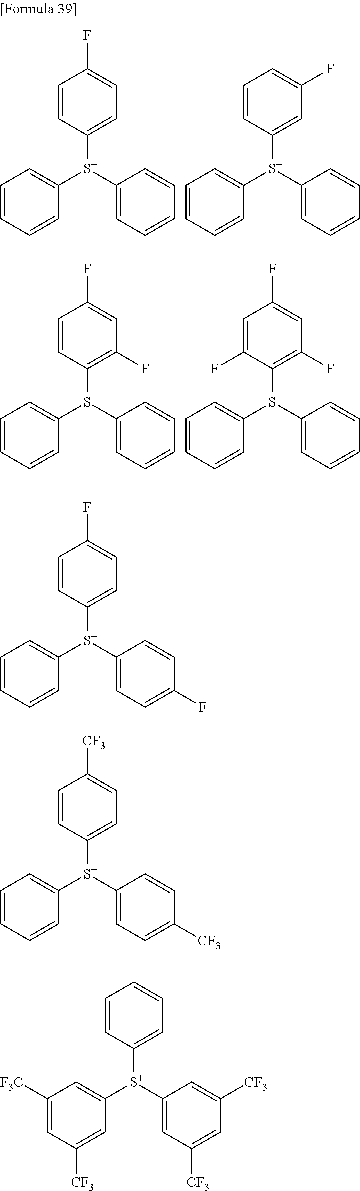

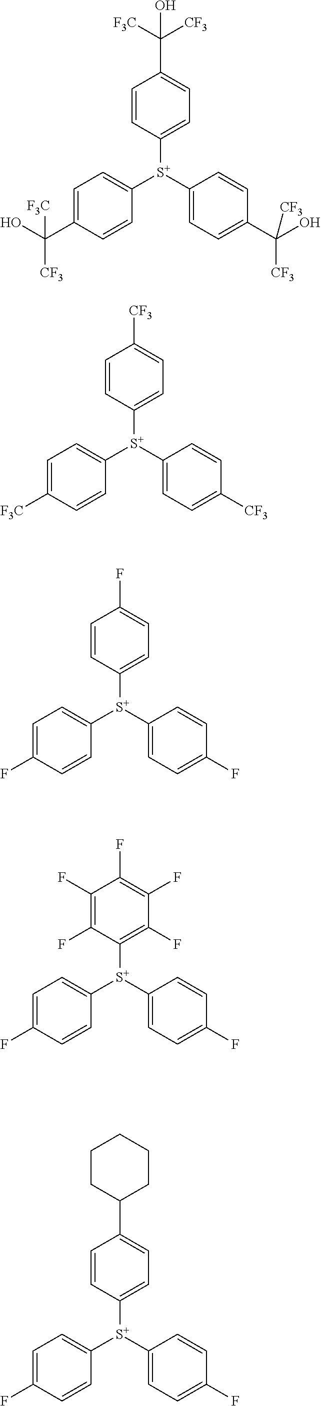 [SMILES: OC(c1ccc([S+](c2ccc(C(O)(C(F)(F)F)C(F)(F)F)cc2)c2ccc(C(O)(C(F)(F)F)C(F)(F)F)cc2)cc1)(C(F)(F)F)C(F)(F)F ; FC(F)(F)c1ccc([S+](c2ccc(C(F)(F)F)cc2)c2ccc(C(F)(F)F)cc2)cc1 ; Fc1ccc([S+](c2ccc(F)cc2)c2ccc(F)cc2)cc1 ; Fc1ccc([S+](c2ccc(F)cc2)c2c(F)c(F)c(F)c(F)c2F)cc1 ; Fc1ccc([S+](c2ccc(F)cc2)c2ccc(C3CCCCC3)cc2)cc1]

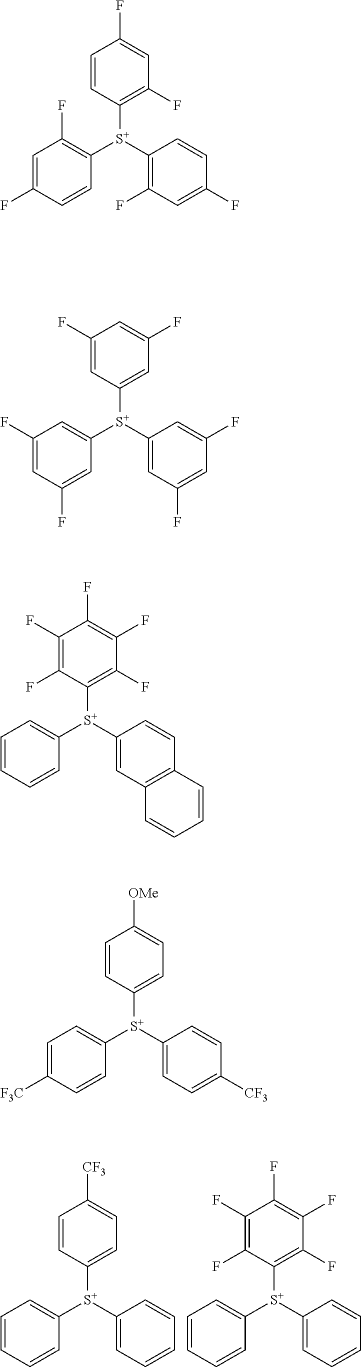 [SMILES: Fc1ccc([S+](c2ccc(F)cc2F)c2ccc(F)cc2F)c(F)c1 ; Fc1cc(F)cc([S+](c2cc(F)cc(F)c2)c2cc(F)cc(F)c2)c1 ; Fc1c(F)c(F)c([S+](c2ccccc2)c2ccc3ccccc3c2)c(F)c1F ; COc1ccc([S+](c2ccc(C(F)(F)F)cc2)c2ccc(C(F)(F)F)cc2)cc1 ; FC(F)(F)c1ccc([S+](c2ccccc2)c2ccccc2)cc1 ; Fc1c(F)c(F)c([S+](c2ccccc2)c2ccccc2)c(F)c1F]

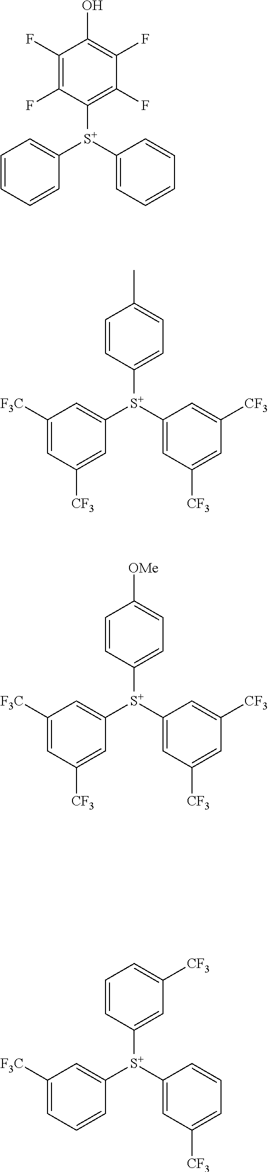 [SMILES: Oc1c(F)c(F)c([S+](c2ccccc2)c2ccccc2)c(F)c1F ; Cc1ccc([S+](c2cc(C(F)(F)F)cc(C(F)(F)F)c2)c2cc(C(F)(F)F)cc(C(F)(F)F)c2)cc1 ; COc1ccc([S+](c2cc(C(F)(F)F)cc(C(F)(F)F)c2)c2cc(C(F)(F)F)cc(C(F)(F)F)c2)cc1 ; FC(F)(F)c1cccc([S+](c2cccc(C(F)(F)F)c2)c2cccc(C(F)(F)F)c2)c1]

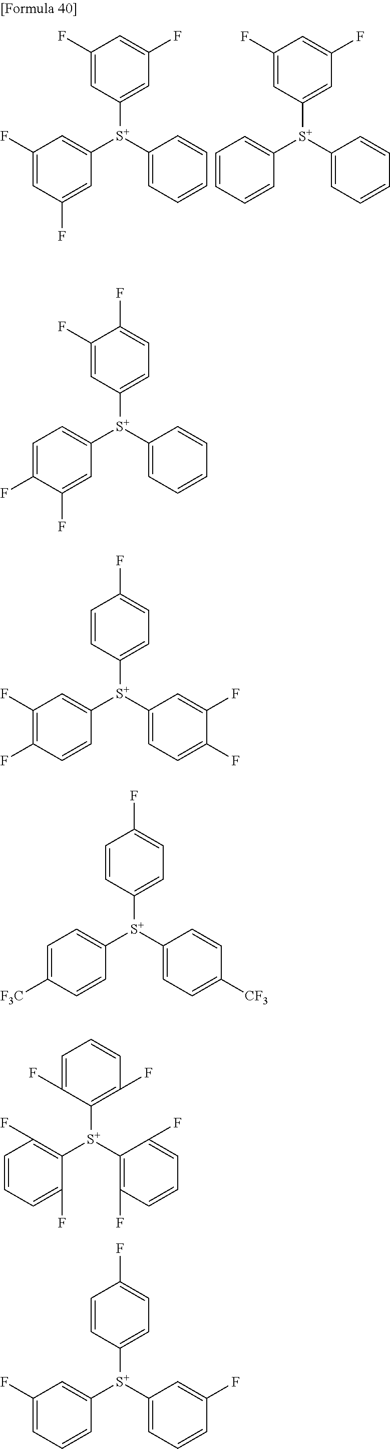

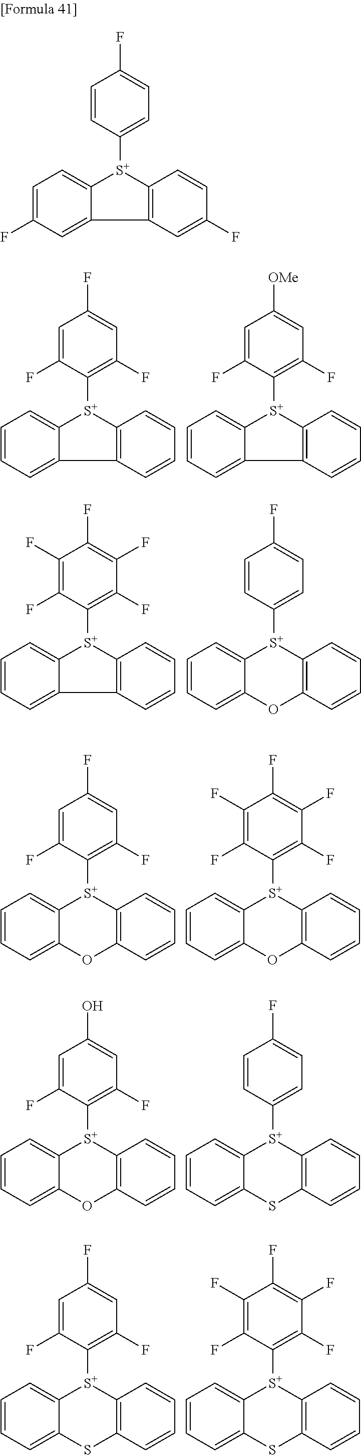

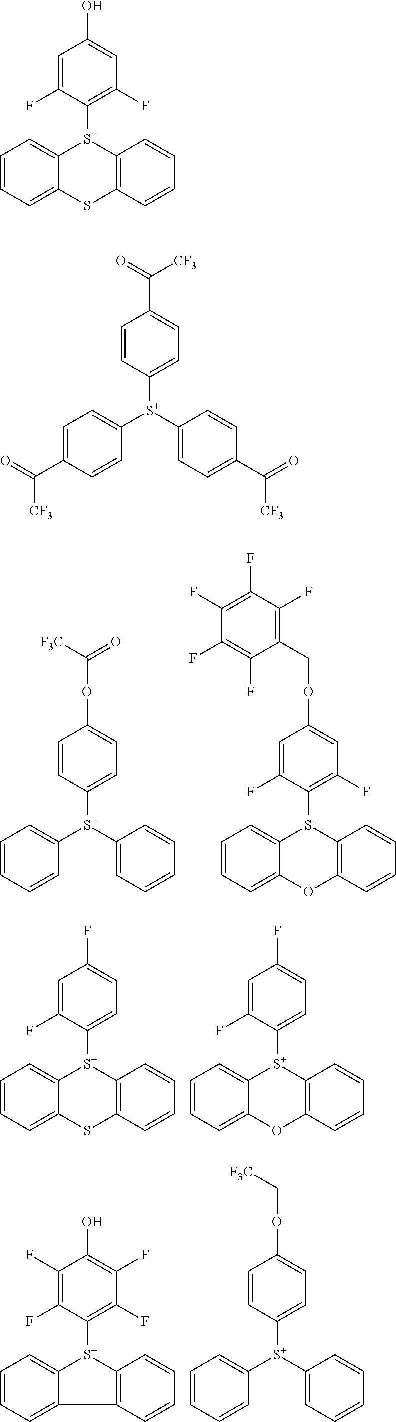

When the onium cation moiety in the radiation-sensitive acid generator represented by the formula (A-2) contains an aromatic ring structure having a fluorine atom, the onium cation moiety is preferably a diaryliodonium cation having one or more fluorine atoms.

Examples of such an onium cation moiety represented by the formula (Q-2) include those shown below. While all of those shown below are iodonium cation moieties containing an aromatic ring structure having a fluorine atom, structures in which a fluorine atom or CF₃ in the following formulas is replaced by an atom or group other than a fluorine atom such as a hydrogen atom or other substituent can be suitably employed as an onium cation moiety containing no aromatic ring structure having a fluorine atom.

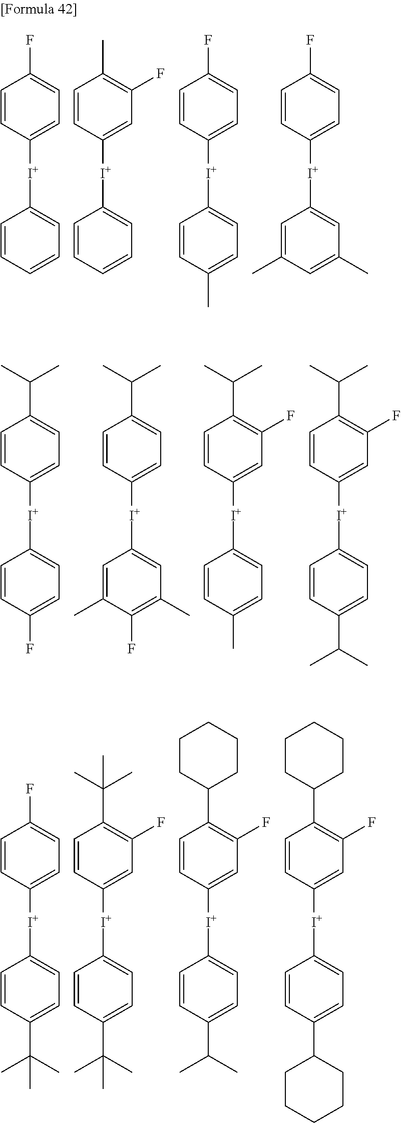

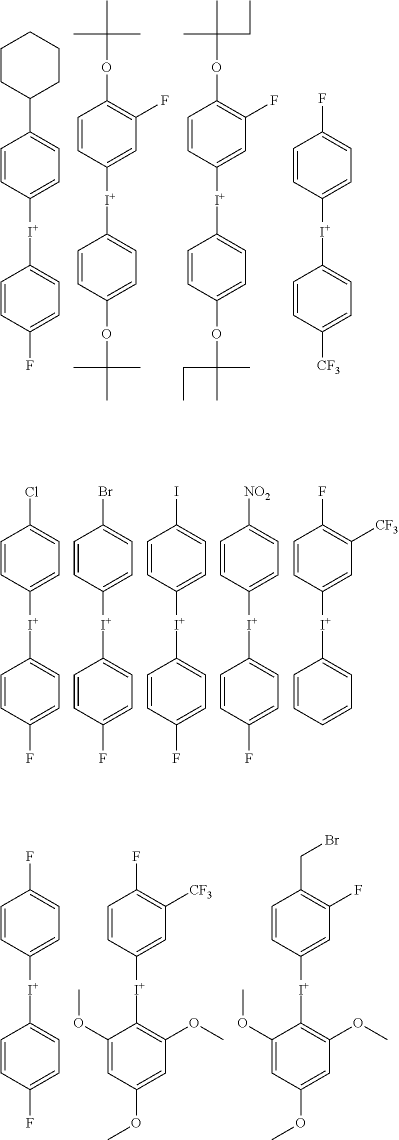

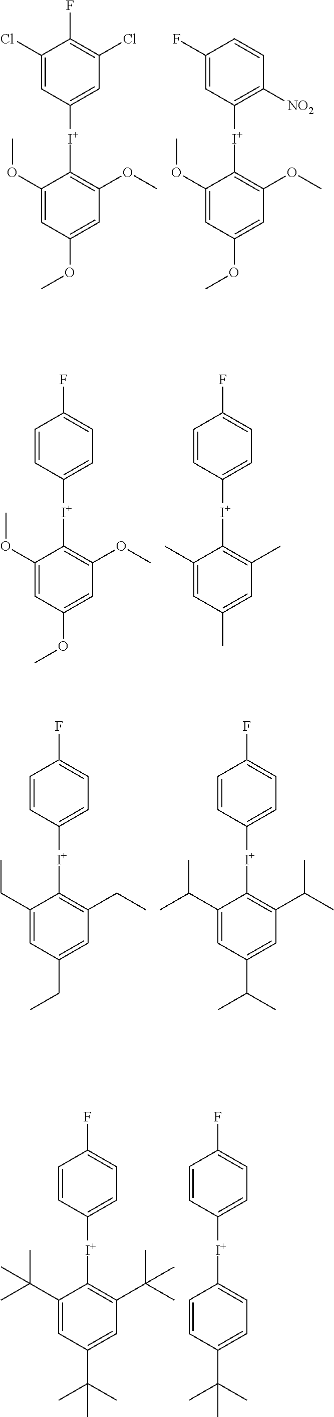

The radiation-sensitive acid generators represented by the above formulas (A-1) and (A-2) can also be synthesized by a known method, particularly by a salt exchange reaction. A known radiation-sensitive acid generator may also be used as long as the effect of the present invention is not impaired.

These radiation-sensitive acid generators may be used singly or two or more of them may be used in combination. The lower limit of the content of the radiation-sensitive acid generator is preferably 0.5 parts by mass, more preferably 1 part by mass, still more preferably 2 parts by mass, and particularly preferably 4 parts by mass based on 100 parts by mass of the base resin. The upper limit of the content is preferably 20 parts by mass, more preferably 18 parts by mass, still more preferably 15 parts by mass, and particularly preferably 12 parts by mass. This makes it possible to exhibit superior sensitivity or CDU performance when forming a resist pattern.

<Acid Diffusion Controlling Agent>

The onium salt as the acid diffusion controlling agent contains an organic acid anion moiety and an onium cation moiety and generates an acid having a higher pKa than an acid to be generated from the radiation-sensitive acid generator through irradiation with radiation. The acid diffusion controlling agent is preferably represented by the following formula (S-1) or (S-2).

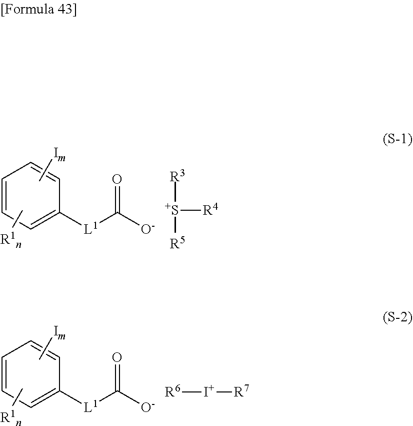

In the formulas (S-1) and (S-2), R¹ is a hydrogen atom, a hydroxy group, a fluorine atom, a chlorine atom, an amino group, a nitro group, or a cyano group; or an alkyl group having 1 to 6 carbon atoms, an alkoxy group having 1 to 6 carbon atoms, an acyloxy group having 2 to 6 carbon atoms, or an alkylsulfonyloxy group having 1 to 4 carbon atoms, which may be substituted with a halogen atom; or —NR^{1A}—C(═O)—R^{1B} or —NR^{1A}—C(═O)—O—R^{1B}. R^{1A} is a hydrogen atom or an alkyl group having 1 to 6 carbon atoms, and R^{1B} is an alkyl group having 1 to 6 carbon atoms or an alkenyl group having 2 to 8 carbon atoms.

R³, R⁴, R⁵, R⁶, and R⁷ are each independently a monovalent hydrocarbon group having 1 to 20 carbon atoms and optionally containing a hetero atom. R³, R⁴, and R⁵ are each preferably a monovalent hydrocarbon group containing one or more fluorine atoms or containing a group having a fluorine atom, and R⁶ and R⁷ are each preferably a monovalent hydrocarbon group containing one or more fluorine atoms or containing a group having a fluorine atom. Any two of R³, R⁴, and R⁵ may be bonded to each other to form a ring together with the sulfur atom to which they are bonded. The monovalent hydrocarbon group may be linear, branched, or cyclic, and examples thereof include an alkyl group having 1 to 12 carbon atoms, an alkenyl group having 2 to 12 carbon atoms, an alkynyl group having 2 to 12 carbon atoms, an aryl group having 6 to 20 carbon atoms, and an aralkyl group having 7 to 12 carbon atoms.

L¹ is a single bond or a divalent linking group having 1 to 20 carbon atoms, and may contain an ether linkage, a carbonyl group, an ester linkage, an amide linkage, a sultone ring, a lactam ring, a carbonate linkage, a halogen atom, a hydroxy group, or a carboxy group.

m and n are integers satisfying 0≤m≤5, 0≤n≤3, and 0≤m+n≤5, and preferably integers satisfying 1≤m≤3 and 0≤n≤2.

Examples of the anion of the acid diffusion controlling agent represented by the above formula (S-1) or (S-2) include, but are not limited to, those shown below. While all of those shown below are organic acid anion moieties having an iodine-substituted aromatic ring structure, organic acid anion moieties having no iodine-substituted aromatic ring structure that can be suitably employed include structures in which the iodine atoms in the formulas shown below are replaced by an atom or group other than an iodine atom such as a hydrogen atom or other substituent.

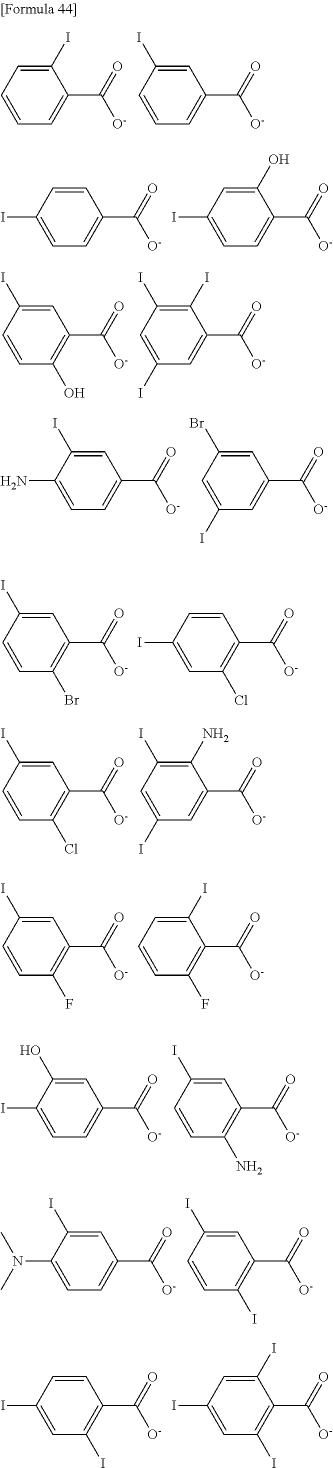

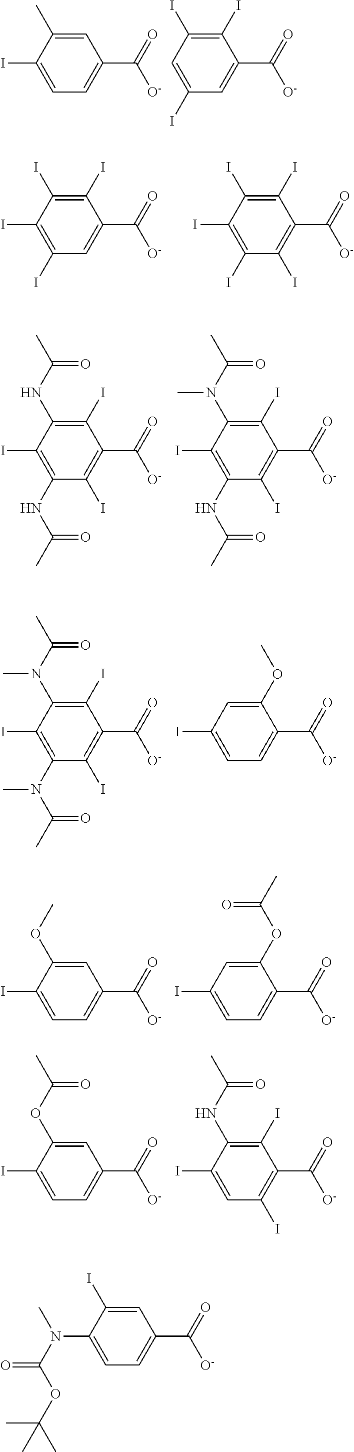

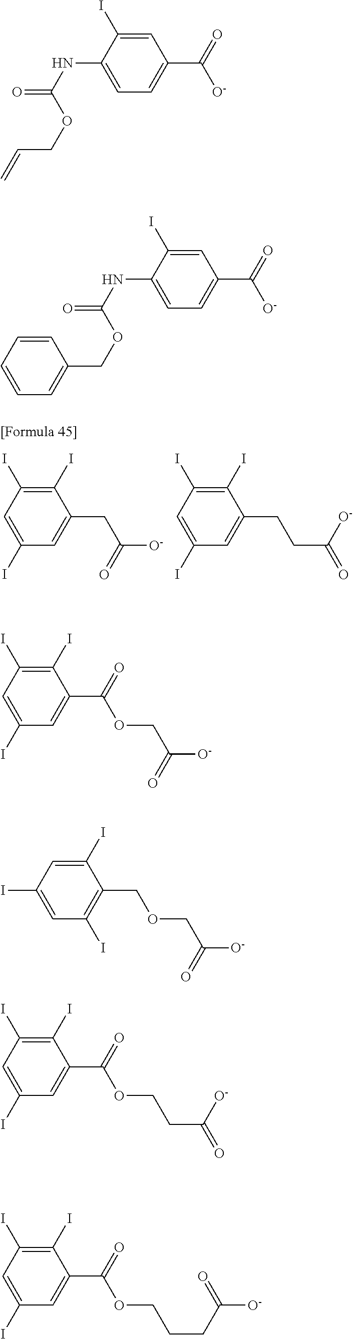

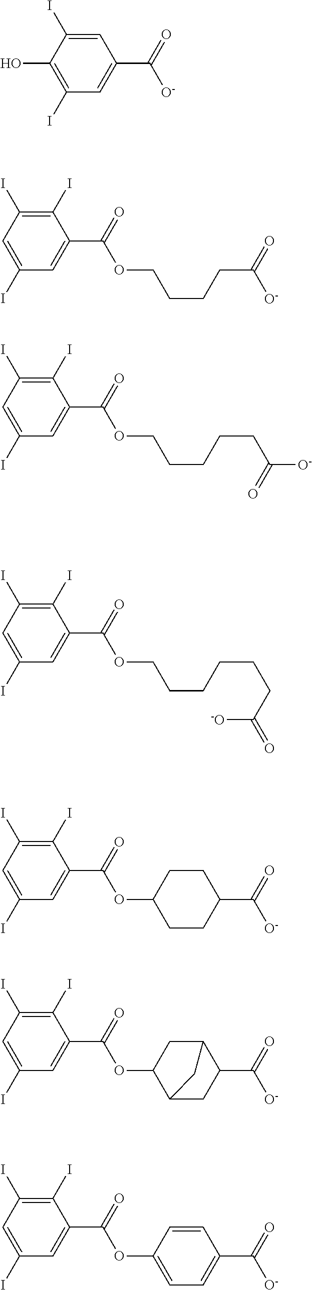

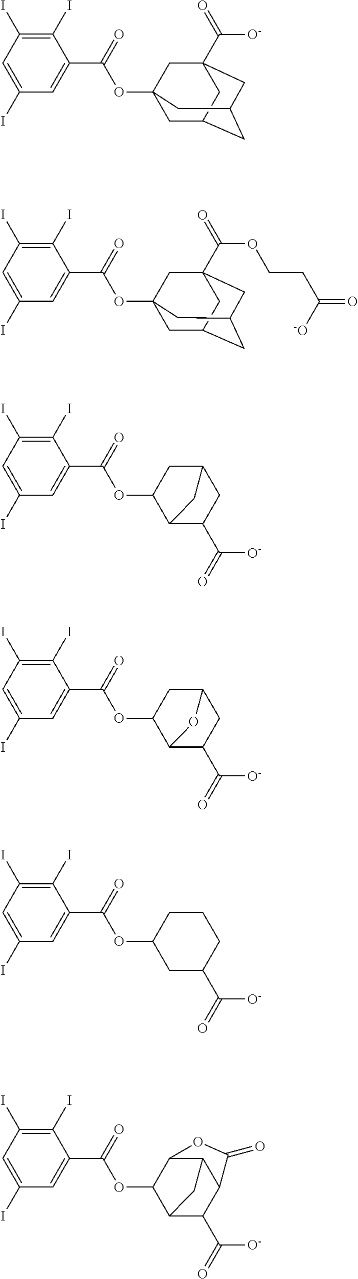

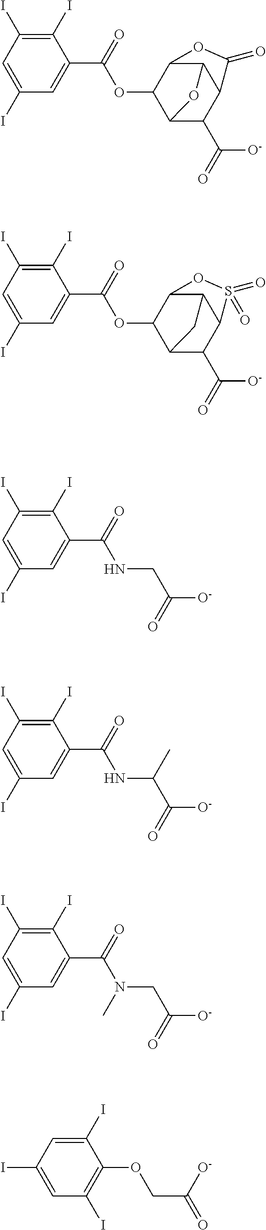 [SMILES: O=C(OC1C2OC(=O)C3C2OC1C3C(=O)[O-])c1cc(I)cc(I)c1I ; O=C(OC1C2CC3C1OS(=O)(=O)C3C2C(=O)[O-])c1cc(I)cc(I)c1I ; O=C([O-])CNC(=O)c1cc(I)cc(I)c1I ; CC(NC(=O)c1cc(I)cc(I)c1I)C(=O)[O-] ; CN(CC(=O)[O-])C(=O)c1cc(I)cc(I)c1I ; O=C([O-])COc1c(I)cc(I)cc1I]

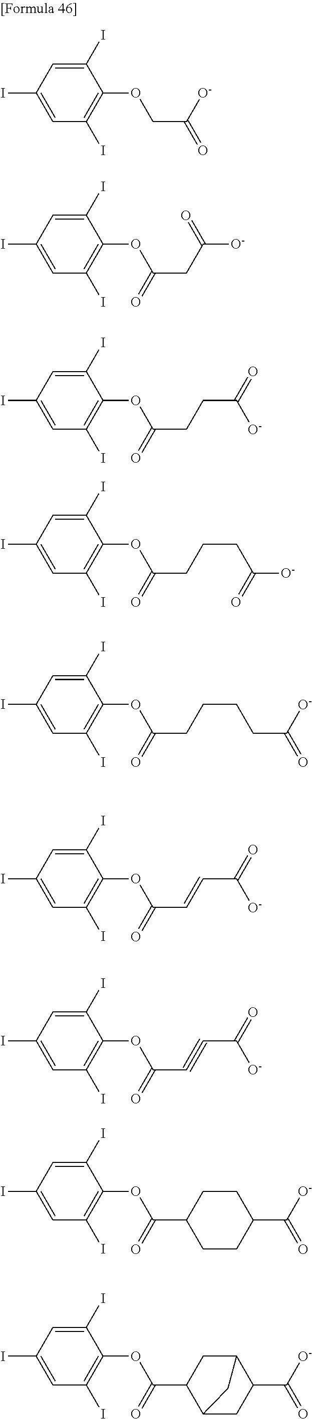

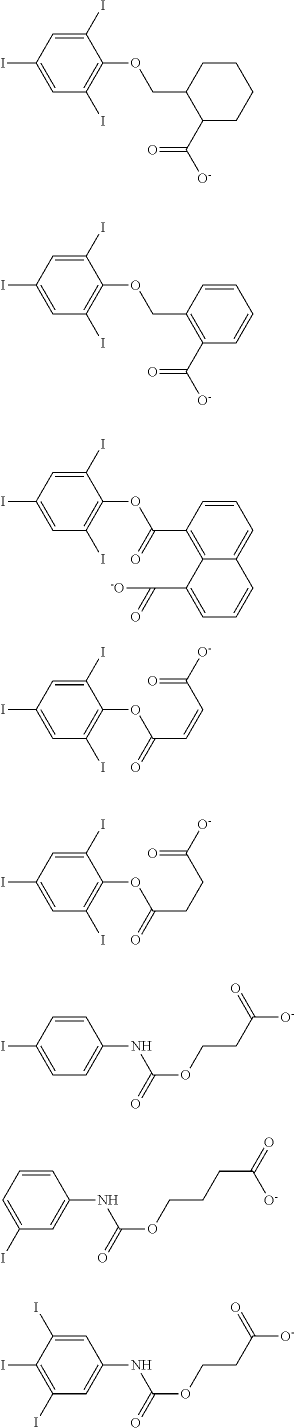

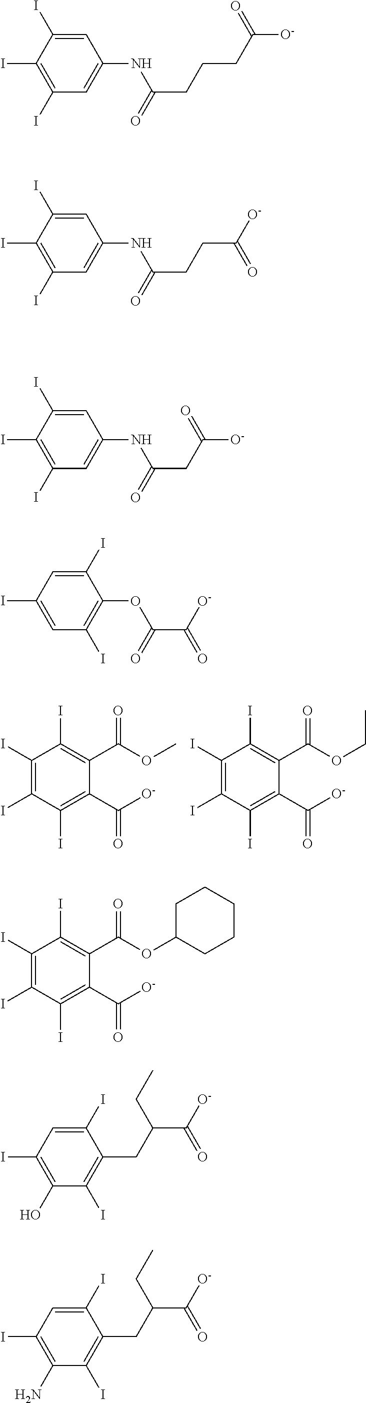

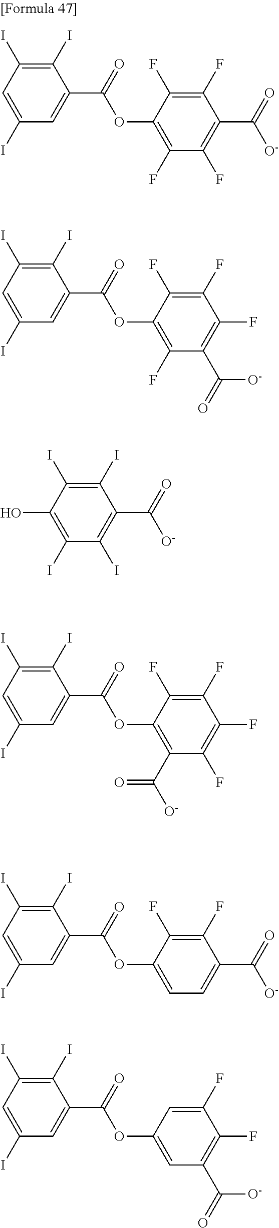 [SMILES: O=C(Oc1c(F)c(F)c(C(=O)[O-])c(F)c1F)c1cc(I)cc(I)c1I ; O=C(Oc1c(F)c(F)c(F)c(C(=O)[O-])c1F)c1cc(I)cc(I)c1I ; O=C([O-])c1c(I)c(I)c(O)c(I)c1I ; O=C(Oc1c(F)c(F)c(F)c(F)c1C(=O)[O-])c1cc(I)cc(I)c1I ; O=C([O-])c1ccc(OC(=O)c2cc(I)cc(I)c2I)c(F)c1F ; O=C([O-])c1cc(OC(=O)c2cc(I)cc(I)c2I)cc(F)c1F]

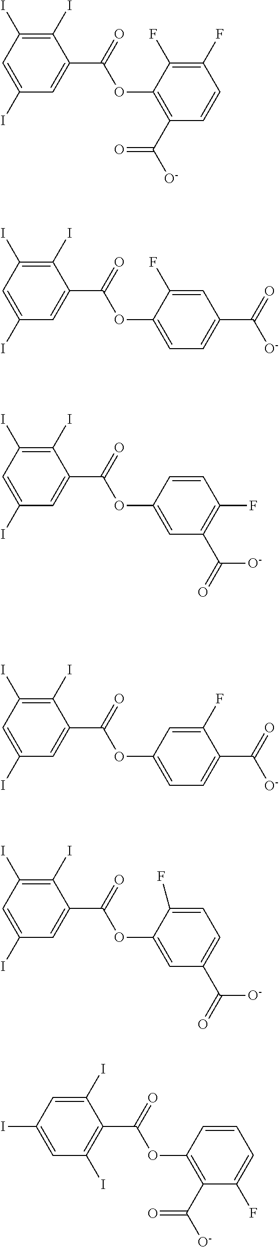 [SMILES: O=C(Oc1c(C(=O)[O-])ccc(F)c1F)c1cc(I)cc(I)c1I ; O=C([O-])c1ccc(OC(=O)c2cc(I)cc(I)c2I)c(F)c1 ; O=C([O-])c1cc(OC(=O)c2cc(I)cc(I)c2I)ccc1F ; O=C([O-])c1ccc(OC(=O)c2cc(I)cc(I)c2I)cc1F ; O=C([O-])c1ccc(F)c(OC(=O)c2cc(I)cc(I)c2I)c1 ; O=C(Oc1cccc(F)c1C(=O)[O-])c1c(I)cc(I)cc1I]

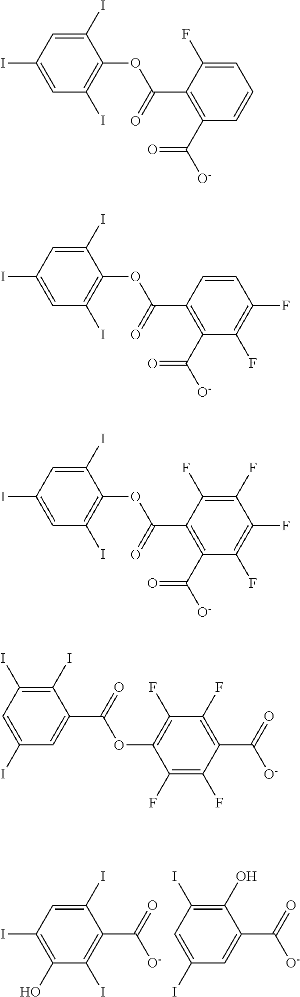 [SMILES: O=C([O-])c1cccc(F)c1C(=O)Oc1c(I)cc(I)cc1I ; O=C(Oc1c(I)cc(I)cc1I)c1ccc(F)c(F)c1C(=O)[O-] ; O=C([O-])c1c(F)c(F)c(F)c(F)c1C(=O)Oc1c(I)cc(I)cc1I ; O=C(Oc1c(F)c(F)c(C(=O)[O-])c(F)c1F)c1cc(I)cc(I)c1I ; O=C([O-])c1c(I)cc(I)c(O)c1I ; O=C([O-])c1cc(I)cc(I)c1O]

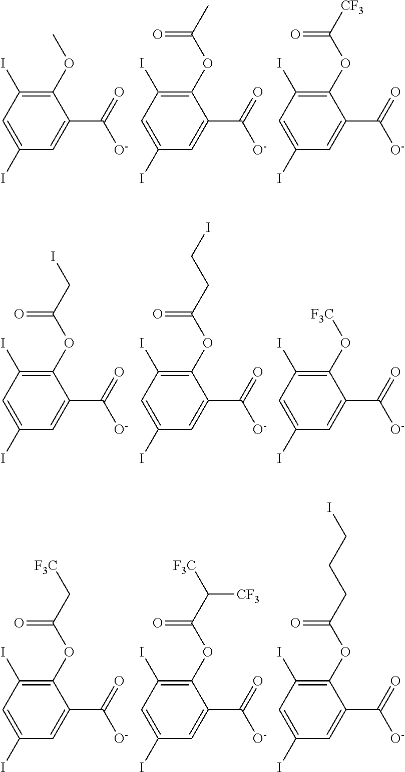

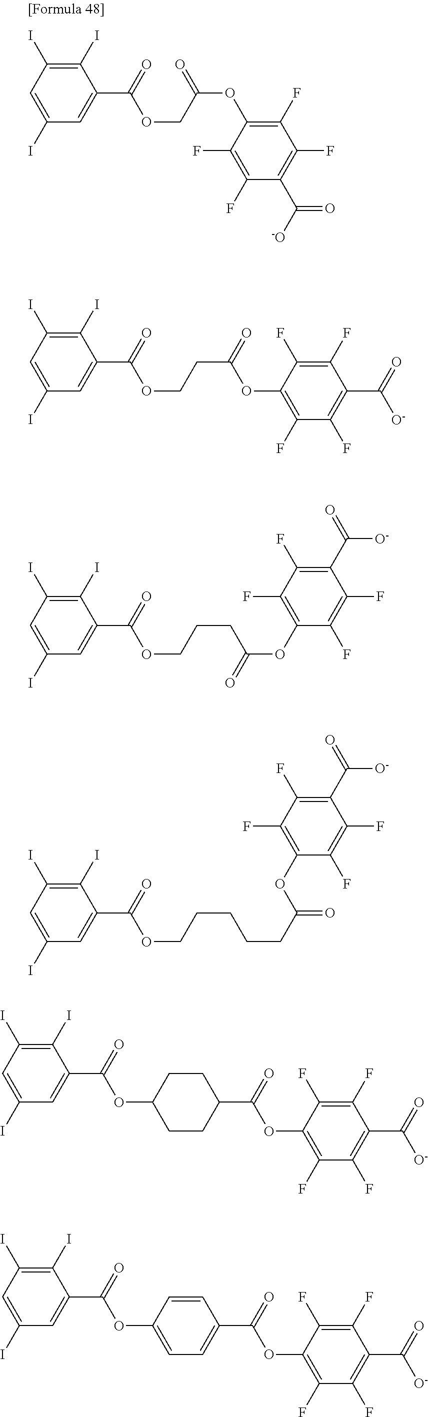

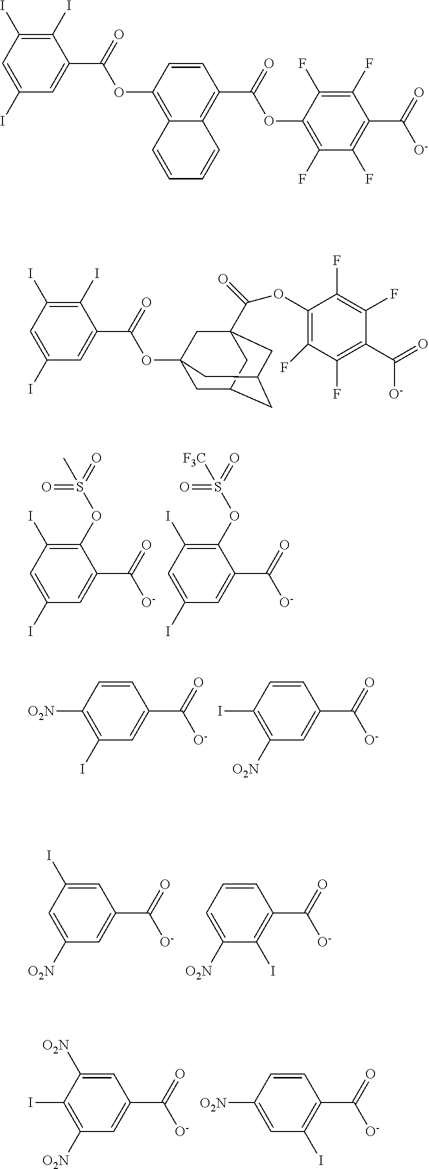

As the onium cation moieties in the acid diffusion controlling agents represented by the formulas (S-1) and (S-2), the onium cation moiety in the radiation-sensitive acid generator can be suitably employed.

The acid diffusion controlling agents represented by the formulas (S-1) and (S-2) can also be synthesized by a known method, particularly by a salt exchange reaction. A known acid diffusion controlling agent may also be used as long as the effect of the present invention is not impaired. In addition, a case where the organic acid anion moiety and the onium cation moiety share the same aromatic ring structure is also included in the acid diffusion controlling agent of the present embodiment.

These acid diffusion controlling agents may be used singly, or two or more thereof may be used in combination. The lower limit of the content of the acid diffusion controlling agent is preferably 0.5 parts by mass, more preferably 1 part by mass, and still more preferably 1.5 parts by mass based on 100 parts by mass of the base resin. The upper limit of the content is preferably 15 parts by mass, more preferably 12 parts by mass, and still more preferably 8 parts by mass. This makes it possible to exhibit superior sensitivity or CDU performance when forming a resist pattern.

<Solvent>

The radiation-sensitive resin composition according to the present embodiment contains a solvent. The solvent is not particularly limited as long as it is a solvent capable of dissolving or dispersing at least an onium salt, a base resin (the radiation-sensitive acid generating resin and at least one of the resins), and additives which are contained as desired.

Examples of the solvent include an alcohol-based solvent, an ether-based solvent, a ketone-based solvent, an amide-based solvent, an ester-based solvent, and a hydrocarbon-based solvent.

Examples of the alcohol-based solvent include:
monoalcohol-based solvents having 1 to 18 carbon atoms, such as iso-propanol, 4-methyl-2-pentanol, 3-methoxybutanol, n-hexanol, 2-ethylhexanol, furfuryl alcohol, cyclohexanol, 3,3,5-trimethylcyclohexanol, and diacetone alcohol;
polyhydric alcohol-based solvents having 2 to 18 carbon atoms, such as ethylene glycol, 1,2-propylene glycol, 2-methyl-2,4-pentanediol, 2,5-hexanediol, diethylene glycol, dipropylene glycol, triethylene glycol, and tripropylene glycol; and
a partially etherized polyhydric alcohol-based solvent resulting from etherification of some of the hydroxy groups of the above-described polyhydric alcohol-based solvent.

Examples of the ether-based solvent include:
dialkyl ether-based solvents, such as diethyl ether, dipropyl ether, and dibutyl ether;
cyclic ether-based solvents, such as tetrahydrofuran and tetrahydropyran;
aromatic ring-containing ether-based solvents, such as diphenyl ether and anisole (methyl phenyl ether); and
a polyhydric alcohol ether-based solvent resulting from etherification of hydroxy groups of the above-described polyhydric alcohol-based solvent.

Examples of the ketone-based solvent include a chain ketone-based solvent, such as acetone, butanone, and methyl-iso-butyl ketone;

cyclic ketone-based solvents, such as cyclopentanone, cyclohexanone, and methylcyclohexanone; and
2,4-pentanedione, acetonylacetone, and acetophenone.

Examples of the amide-based solvent include a cyclic amide-based solvent, such as N,N′-dimethylimidazolidinone and N-methylpyrrolidone; and
a chain amide-based solvent, such as N-methylformamide, N,N-dimethylformamide, N,N-diethylformamide, acetamide, N-methylacetamide, N,N-dimethylacetamide, and N-methylpropionamide.

Examples of the ester-based solvent include:
monocarboxylate ester-based solvents, such as n-butyl acetate and ethyl lactate;
partially etherized polyhydric alcohol acetate-based solvents, such as diethylene glycol mono-n-butyl ether acetate, propylene glycol monomethyl ether acetate, and dipropylene glycol monomethyl ether acetate;
lactone-based solvents, such as γ-butyrolactone and valerolactone;
carbonate-based solvents, such as diethyl carbonate, ethylene carbonate, and propylene carbonate; and
a polyhydric carboxylic acid diester-based solvent, such as propylene glycol diacetate, methoxy triglycol acetate, diethyl oxalate, ethyl acetoacetate, ethyl lactate, and diethyl phthalate.

Examples of the hydrocarbon-based solvent include:
aliphatic hydrocarbon-based solvents, such as n-hexane, cyclohexane, and methylcyclohexane; and
an aromatic hydrocarbon-based solvent, such as benzene, toluene, di-iso-propylbenzene, and n-amylnaphthalene.

Among them, ester-based solvents and ketone-based solvents are preferable, polyhydric alcohol partial ether acetate-based solvents, cyclic ketone-based solvents, and lactone-based solvents are more preferable, and propylene glycol monomethyl ether acetate, cyclohexanone, and γ-butyrolactone are still more preferable. The radiation-sensitive resin composition may contain one or two or more solvents.

<Other Optional Components>

The radiation-sensitive resin composition may contain other optional components in addition to the components described above. Examples of the other optional components include a crosslinking agent, a localization enhancing agent, a surfactant, an alicyclic backbone-containing compound, and a sensitizer. Such other optional components may be used singly or two or more types thereof may be used in combination.

<Method for Preparing Radiation-Sensitive Resin Composition>

The radiation-sensitive resin composition can be prepared, for example, by mixing an onium salt, a base resin (at least one of a radiation-sensitive acid generating resin and a resin) and a solvent, and if necessary, other optional component at a prescribed ratio. The radiation-sensitive resin composition is preferably filtered through, for example, a filter having a pore size of approximately 0.05 μm to 0.2 μm after mixing. The solid concentration of the radiation-sensitive resin composition is usually from 0.1% by mass to 50% by mass, preferably from 0.5% by mass to 30% by mass, and more preferably from 1% by mass to 20% by mass.

<Method for Forming Pattern>

The method for forming a resist pattern according to the present invention comprises:
step (1) of applying the radiation-sensitive resin composition directly or indirectly on a substrate to form a resist film (hereinafter also referred to as “resist film forming step”);
step (2) of exposing the resist film to light (hereinafter also referred to as “exposure step”); and
step (3) of developing the exposed resist film with a developer (hereinafter also referred to as “development step”).

In accordance with this method for forming a pattern, a high-quality resist pattern can be formed because of the use of the radiation-sensitive resin composition superior in sensitivity and CDU performance in an exposure step. Hereinbelow, each of the steps will be described. [Resist film forming step]

In this step (step (1)), a resist film is formed from the radiation-sensitive resin composition. Examples of the substrate on which the resist film is formed include conventionally known substrates such as a silicon wafer, silicon dioxide, and a wafer coated with aluminum. An organic or inorganic antireflective film disclosed in, for example, JP-B-6-12452 or JP-A-59-93448 may be formed on the substrate. Examples of a method for applying the composition include spin coating, cast coating, and roll coating. After the application, prebaking (PB) may be performed to volatilize the solvent in the coating film, as necessary. The PB temperature is usually 60° C. to 140° C., and preferably 80° C. to 120° C. The PB time is usually 5 seconds to 600 seconds, and preferably 10 seconds to 300 seconds. The thickness of the resist film to be formed is preferably 10 nm to 1,000 nm, and more preferably 10 nm to 500 nm.

In the case of performing immersion exposure, regardless of the presence or absence of a water repellent polymer additive such as the high-fluorine-containing resin in the radiation-sensitive resin composition, a protective film for immersion insoluble in an immersion liquid may be provided on the formed resist film for the purpose of avoiding direct contact between the immersion liquid and the resist film. As the protective film for immersion, either a solvent-removable protective film that is to be removed by a solvent before the development step (see, for example, JP-A-2006-227632) or a developer-removable protective film that is to be removed simultaneously with the development in the development step (see, for example, WO 2005 069076 and WO 2006 035790) may be used. However, from the viewpoint of throughput, it is preferable to use a developer-removable protective film for immersion.

When the subsequent exposure step is performed with radiation having a wavelength of 50 nm or less, it is preferable to use a resin having the structural units (I) to (IV) and, as necessary, the structural unit (V) as the base resin in the composition.

[Exposure Step]

In this step (the step (2)), the resist film formed in the resist film forming step, namely the step (1), is irradiated with radiation through a photomask (as the case may be, through an immersion medium such as water) to be exposed. Examples of the radiation to be used for the exposure include an electromagnetic wave including visible ray, ultraviolet ray, far ultraviolet ray, extreme ultraviolet ray (EUV), X ray, and γ ray; an electron beam; and a charged particle radiation such as α ray. Among them, far ultraviolet ray, electron beam, and EUV are preferable, ArF excimer laser light (wavelength: 193 nm), KrF excimer laser light (wavelength: 248 nm), electron beam, and EUV are more preferable, and an electron beam and EUV having a wavelength of 50 nm or less, which are positioned as next-generation exposure technology, are still more preferable.

When the exposure is performed by immersion exposure, examples of the immersion liquid to be used include water and a fluorine-based inert liquid. The immersion liquid is preferably a liquid that is transparent to an exposure wavelength and has a temperature coefficient of refractive index as small as possible to minimize the distortion of an optical image projected onto the film. Particularly, when an exposure light source is ArF excimer laser light (wavelength: 193 nm), water is preferably used from the viewpoint of availability and ease of handling in addition to the above-described viewpoints. When water is used, an additive that reduces the surface tension of water and increases the surface activity may be added in a small proportion. This additive is preferably one that does not dissolve the resist film on a wafer and has negligible influence on an optical coating at an under surface of a lens. The water to be used is preferably distilled water.

After the exposure, post exposure baking (PEB) is preferably carried out to promote the dissociation of the acid-dissociable group of the resin or the like due to the acid generated from the radiation-sensitive acid generator through the exposure in the exposed area of the resist film. As a result of the PEB, there is produced a difference in solubility in the developer between the exposed area and the unexposed area. The PEB temperature is usually 50° C. to 180° C., and preferably 80° C. to 130° C. The PEB time is usually 5 seconds to 600 seconds, and preferably 10 seconds to 300 seconds.

[Development step]

In this step (the step (3)), the resist film exposed in the exposure step, namely the step (2), is developed with a developer. Thus, a prescribed resist pattern can be formed. In a common procedure, after the development, the film is washed with a rinsing liquid such as water or alcohol and dried.

Examples of the developer to be used for the development include, in the alkaline development, an alkaline aqueous solution obtained by dissolving at least one alkaline compound such as sodium hydroxide, potassium hydroxide, sodium carbonate, sodium silicate, sodium metasilicate, ammonia water, ethylamine, n-propylamine, diethylamine, di-n-propylamine, triethylamine, methyldiethylamine, ethyldimethylamine, triethanolamine, tetramethyl ammonium hydroxide (TMAH), pyrrole, piperidine, choline, 1,8-diazabicyclo-[5.4.0]-7-undecene, and 1,5-diazabicyclo-[4.3.0]-5-nonene. Among them, the aqueous TMAH solution is preferable, and a 2.38% by mass aqueous TMAH solution is more preferable.

In the case of organic solvent development, examples of the solvent include organic solvents such as hydrocarbon-based solvents, ether-based solvents, ester-based solvents, ketone-based solvents, and alcohol-based solvents, and solvents containing an organic solvent. Examples of the organic solvent include one or two or more solvents among the solvents listed as the solvent for the radiation-sensitive resin composition. Among them, ester-based solvents and ketone-based solvents are preferable. As the ester-based solvents, acetate-based solvents are preferable, and n-butyl acetate and amyl acetate are more preferable. As the ketone-based solvents, chain ketones are preferable, and 2-heptanone is more preferable. The content of the organic solvent in the developer is preferably 80% by mass or more, more preferably 90% by mass or more, still more preferably 95% by mass, and particularly preferably 99% by mass. Examples of the components other than the organic solvent in the developer include water and silicon oil.

Examples of a development method include a method in which a substrate is immersed in a bath filled with a developer for a certain period of time (dipping method), a method in which a developer is allowed to be present on a surface of a substrate due to surface tension and to stand for a certain period of time (puddle method), a method in which a developer is sprayed onto a surface of a substrate (spray method), and a method in which a developer is discharged onto a substrate that is rotated at a constant speed while a developer discharge nozzle is scanned at a constant speed (dynamic dispensing method).

### Examples

Hereinafter, the present invention will specifically be described with reference to synthesis examples, examples, and comparative examples, but is not limited to the following examples. Methods for measuring various physical property values will be described below.

[Mw and Mn]

The Mw and the Mn of polymers were measured by gel permeation chromatography (GPC) using GPC columns manufactured by Tosoh Corporation (“G2000HXL”×2, “G3000HXL”×1, “G4000HXL”×1) under the following conditions.
Eluant: tetrahydrofuran (manufactured by Wako Pure Chemical Industries, Ltd.)
Flow rate: 1.0 mL/min
Sample concentration: 1.0% by mass
Sample injection amount: 100 μL
Column temperature: 40° C.
Detector: differential refractometer
Standard substance: monodisperse polystyrene

The structures of the radiation-sensitive acid generators PAG1 to PAG9 of the sulfonium salts or iodonium salts used in the radiation-sensitive resin compositions of Examples are shown below.

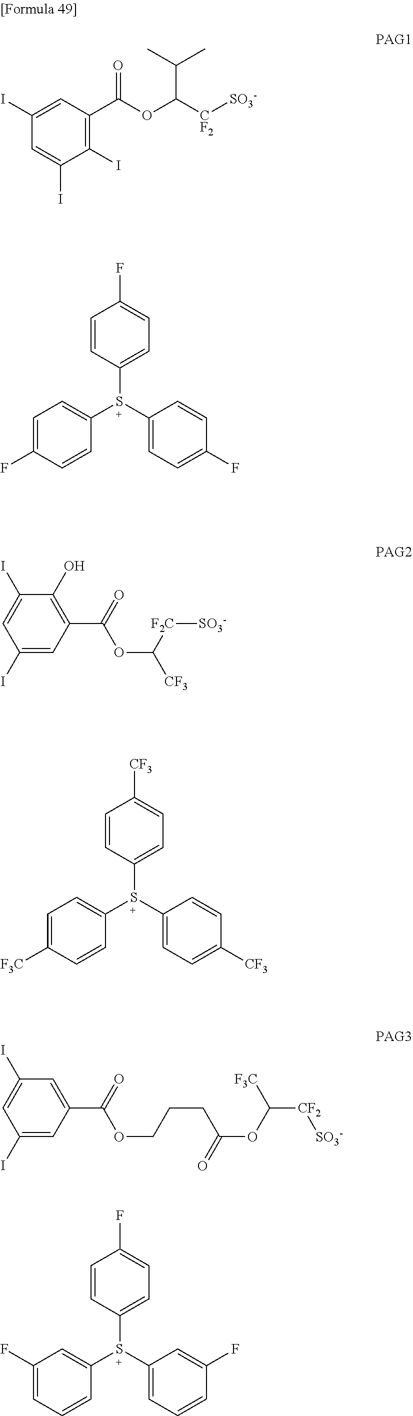

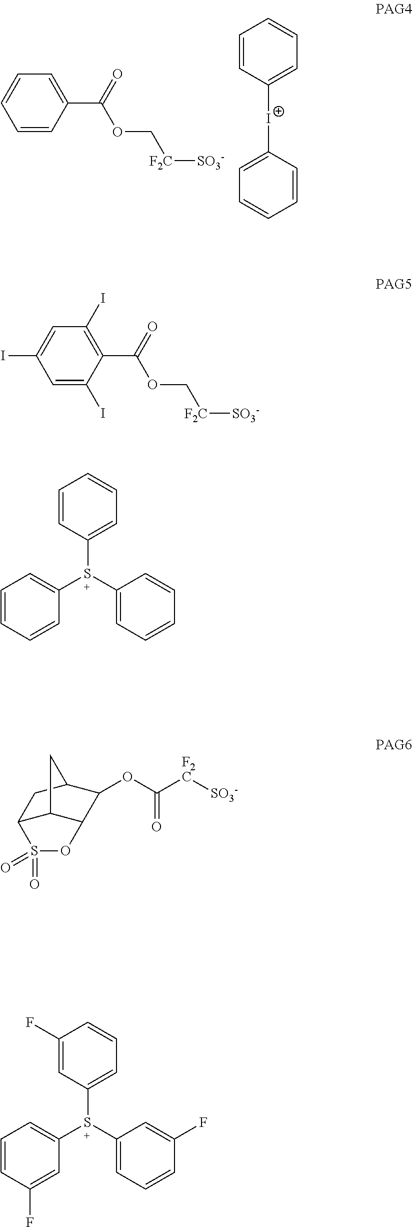

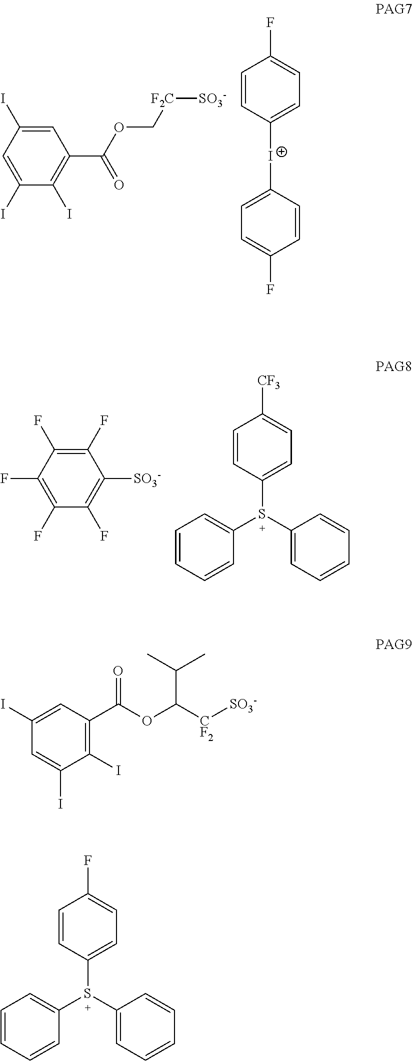

### [Synthesis Example] Synthesis of Base Polymers (P-1) to (P-8)

The respective monomers were combined and subjected to a copolymerization reaction in a tetrahydrofuran (THF) solvent, and the reaction products were crystallized in methanol, and washed repeatedly with hexane, then isolated, and dried. Thus, base polymers (P-1) to (P-8) having the compositions shown below were obtained. The composition of the obtained base polymers was confirmed by ¹H-NMR, and the Mw and the dispersion degree (Mw/Mn) were confirmed by the above-described GPC (solvent: THF, standard: polystyrene).
P-1: Mw=7,800, Mw/Mn=1.6
P-2: Mw=7,200, Mw/Mn=1.7
P-3: Mw=8,300, Mw/Mn=1.6
P-4: Mw=8,200, Mw/Mn=1.6
P-5: Mw=7,700, Mw/Mn=1.7
P-6: Mw=7,000, Mw/Mn=1.7
P-7: Mw=8,500, Mw/Mn=1.7
P-8: Mw=9,000, Mw/Mn=1.6

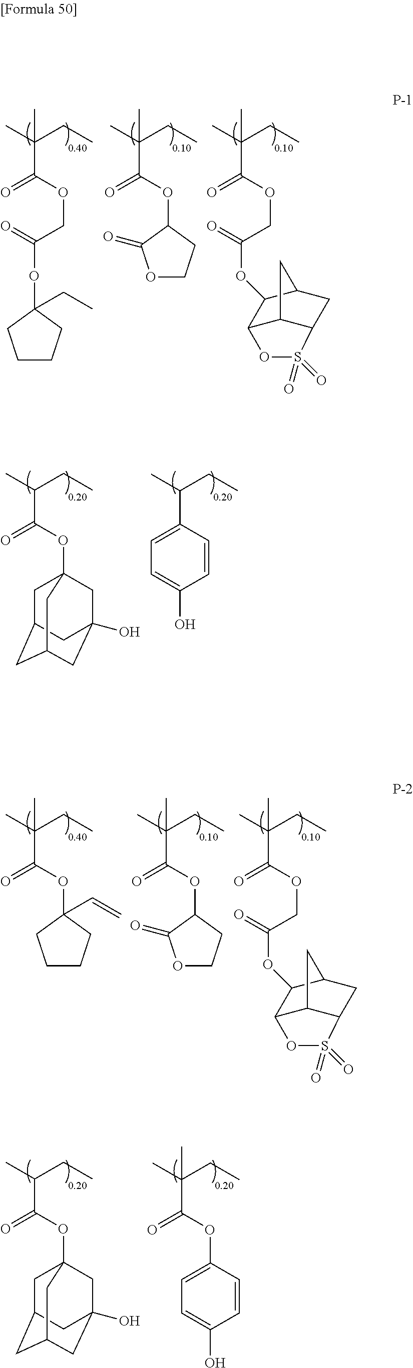

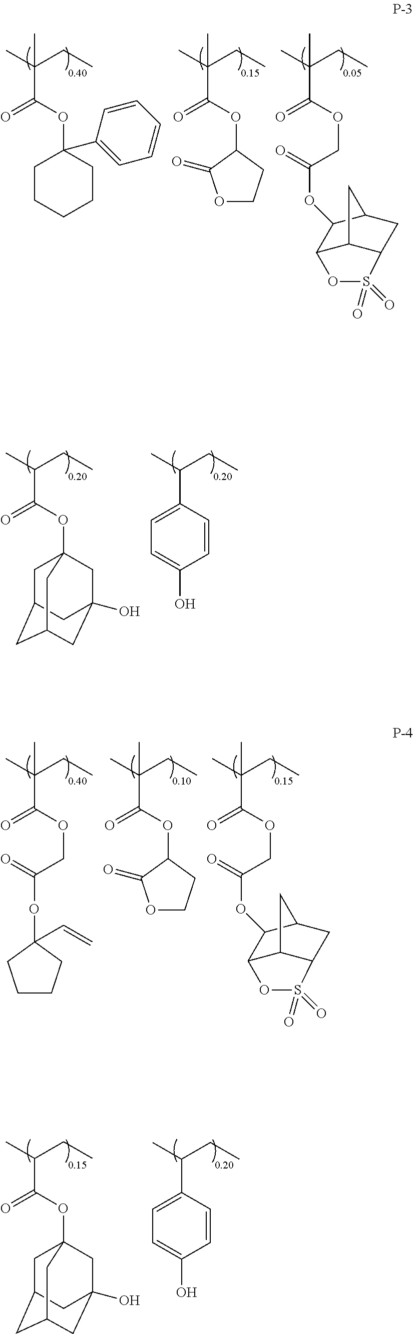

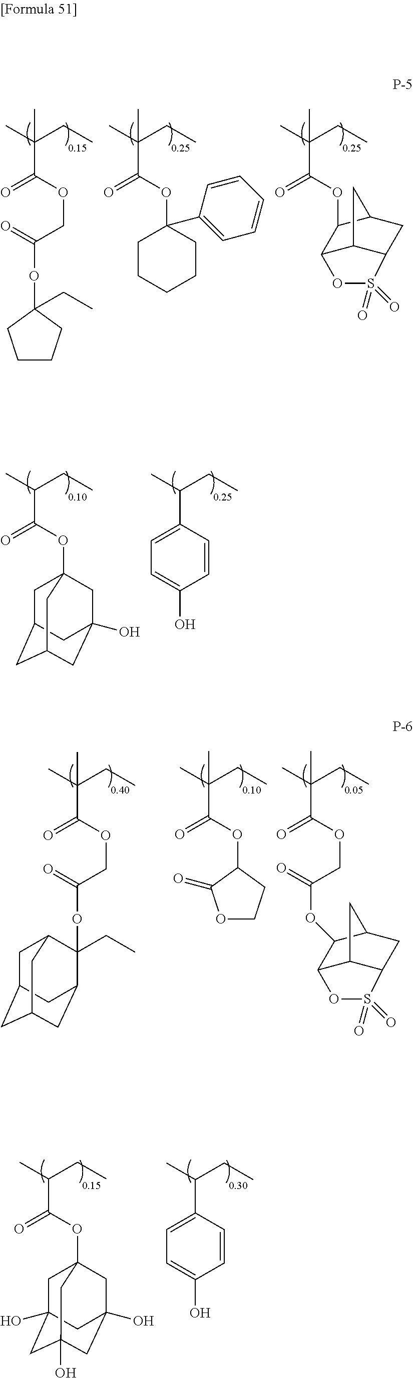

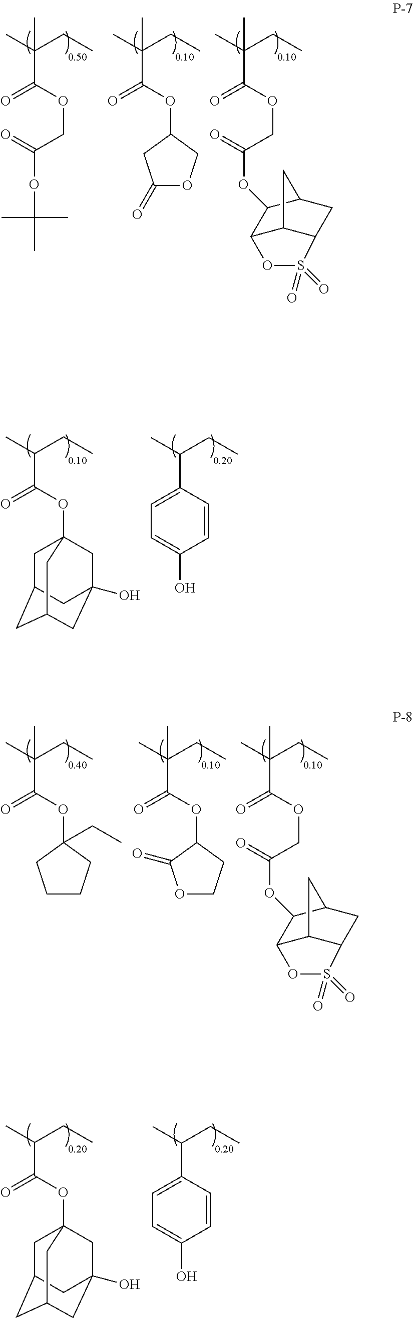

### Examples and Comparative Examples

A radiation-sensitive resin composition was prepared by filtering a solution obtained by dissolving components in the composition given in Table 1 in a solvent obtained by dissolving 100 ppm of FC-4430 manufactured by 3M as a surfactant through a 0.2 μm-sized filter.

In Table 1, the components are as follows.
Organic solvent: PGMEA (propylene glycol monomethyl ether acetate)
GBL (γ-butyrolactone)
CHN (cyclohexanone)
PGME (propylene glycol monomethyl ether)
DAA (diacetone alcohol)
EL (ethyl lactate)

Acid diffusion controlling agents (Q-1) to (Q-6)

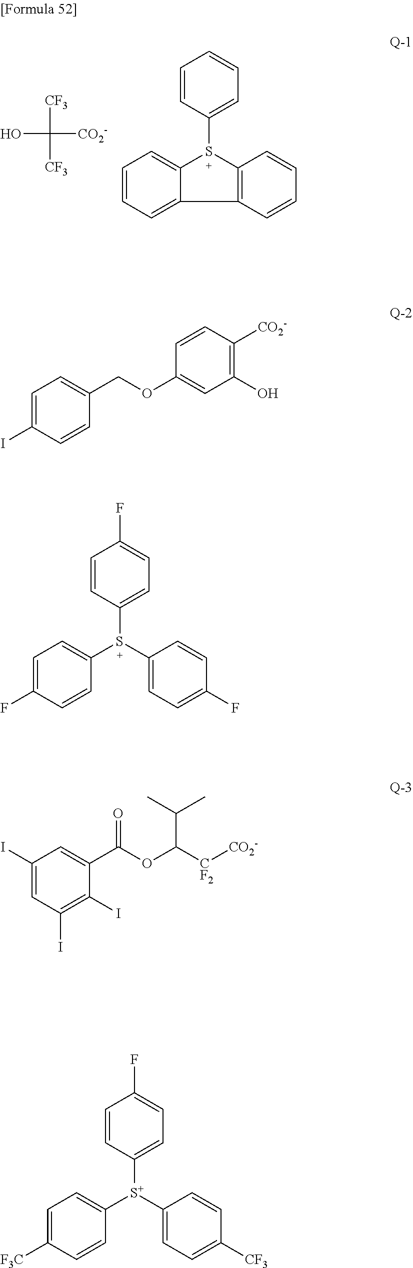

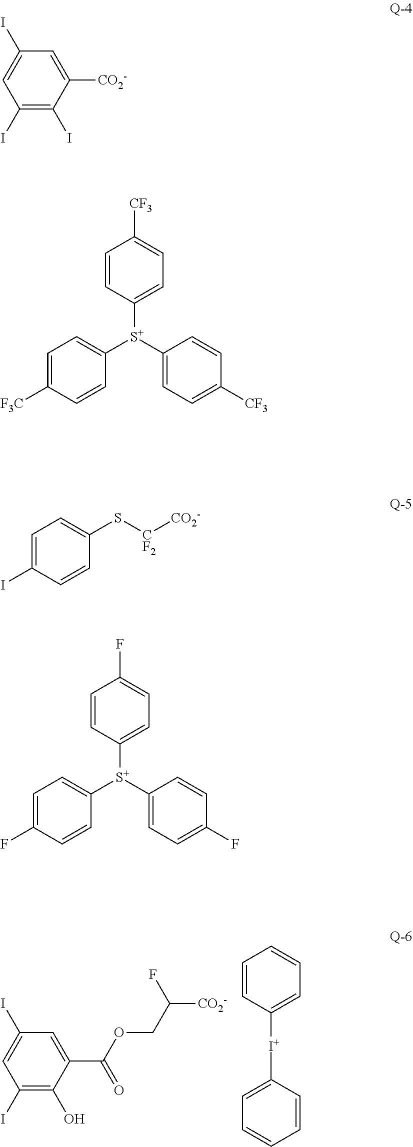

High-fluorine-containing resin F-1: Mw=8,900, Mw/Mn=2.0

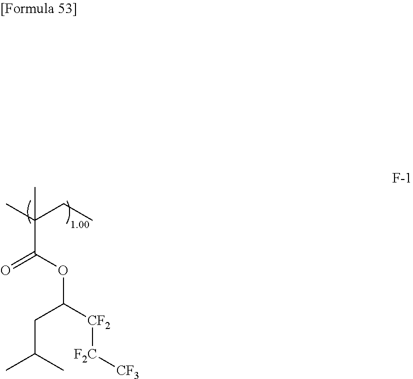

[Evaluation of Sensitivity by EUV Exposure]

Onto the surface of a 12-inch silicon wafer, an underlayer antireflection film forming composition (“ARC66” manufactured by Brewer Science Incorporated) was applied with use of a spin coater (“CLEAN TRACK ACT12” manufactured by Tokyo Electron Limited). The wafer was then heated at 205° C. for 60 seconds to form an underlayer antireflection film having an average thickness of 105 nm. Each radiation-sensitive resin composition shown in Table 1 was applied onto the underlayer antireflection film using the spin coater, followed by performing PB at 130° C. for 60 seconds. Thereafter, cooling was performed at 23° C. for 30 seconds to form a resist film having an average thickness of 55 nm. This resist film was exposed to light using an EUV scanner (“NXE3300” (NA 0.33, σ 0.9/0.6, quadrupole illumination, hole pattern mask with a pitch of 46 nm on wafer and a bias of +20%) manufactured by ASML). PEB was performed on a hot plate at 120° C. for 60 seconds, and development was performed with a 2.38 mass % aqueous tetramethylammonium hydroxide (TMAH) solution for 30 seconds to form a resist pattern with a 23 nm hole and a 46 nm pitch. The amount of light exposure at which the resist pattern with a 23 nm hole and a 46 nm pitch was formed was defined as an optimum amount of light exposure, and the optimum amount of light exposure (Eop) was defined as sensitivity (mJ/cm²).

[Evaluation of CDU]

A resist pattern with a 23 nm hole and a 46 nm pitch was formed by through the same operation as that described above by applying the amount of light exposure Eop determined above. The resist pattern formed was observed from the top of the pattern using a scanning electron microscope (“CG-5000” manufactured by Hitachi High-Technologies Corporation). The hole diameter was measured at 16 points in a range of 500 nm and the average value thereof was determined. In addition, the average value was measured at arbitrary 500 points in total. The 3 sigma value was determined from the distribution of the measurement values, and the 3 sigma value determined was taken as an evaluation value (nm) of CDU performance. The smaller an evaluation value of CDU performance is, the smaller the dispersion of hole diameter in a long period is and the better the CDU performance is. The results are shown in Table 1.

TABLE 1

| | Base resin | PAG | Acid diffusion | Solvent | Additive | | |
|---|---|---|---|---|---|---|---|
| | (parts by | (parts by | controlling agent | (parts by | (parts by | Sensitivity | CDU |
| | mass) | mass) | (parts by mass) | mass) | mass) | [mJ/cm²] | [nm] |
| Example 1 | P-1 | PAG1 | Q-1 | PGMEA/GBL | F-1 | 13 | 2.2 |
| | (100) | (6.5) | (2.5) | (2,200/300) | (3.0) | | |
| Example 2 | P-1 | PAG2 | Q-1 | PGMEA/GBL | F-1 | 13 | 2.2 |
| | (100) | (6.5) | (2.5) | (2,200/300) | (3.0) | | |
| Example 3 | P-1 | PAG3 | Q-1 | PGMEA/GBL | F-1 | 13 | 2.2 |
| | (100) | (6.5) | (2.5) | (2,200/300) | (3.0) | | |
| Example 4 | P-1 | PAG4 | Q-2 | PGMEA/GBL | F-1 | 13 | 2.2 |
| | (100) | (6.5) | (2.5) | (2,200/300) | (3.0) | | |
| Example 5 | P-1 | PAG4 | Q-3 | PGMEA/GBL | F-1 | 13 | 2.2 |
| | (100) | (6.5) | (2.5) | (2,200/300) | (3.0) | | |
| Example 6 | P-1 | PAG4 | Q-4 | PGMEA/GBL | F-1 | 13 | 2.2 |
| | (100) | (6.5) | (2.5) | (2,200/300) | (3.0) | | |
| Example 7 | P-1 | PAG5 | Q-5 | PGMEA/GBL | F-1 | 13 | 2.2 |
| | (100) | (6.5) | (2.5) | (2,200/300) | (3.0) | | |
| Example 8 | P-2 | PAG6 | Q-6 | PGME/EL | F-1 | 13 | 2.2 |
| | (100) | (6.5) | (3.0) | (2,000/500) | (3.0) | | |
| Example 9 | P-3 | PAG4 | Q-2 | PGMEA/EL | F-1 | 13 | 2.2 |
| | (100) | (6.0) | (3.0) | (2,000/500) | (3.0) | | |
| Example 10 | P-4 | PAG1 | Q-1 | PGMEA/GBL | F-1 | 12 | 2.2 |
| | (100) | (6.0) | (2.5) | (2,200/300) | (3.0) | | |
| Example 11 | P-5 | PAG6 | Q-6 | PGMEA/EL | F-1 | 12 | 2.2 |
| | (100) | (6.0) | (2.5) | (2,000/500) | (3.0) | | |
| Example 12 | P-6 | PAG7 | Q-1 | PGMEA/DAA | F-1 | 13 | 2.2 |
| | (100) | (5.5) | (2.5) | (2,000/500) | (3.0) | | |
| Example 13 | P-7 | PAG8 | Q-6 | PGMEA/PGME/EL | F-1 | 14 | 2.2 |
| | (100) | (5.5) | (2.0) | (1,500/500/500) | (3.0) | | |
| Comparative | P-1 | PAG6 | Q-1 | PGMEA/GBL | F-1 | 13 | 2.4 |
| Example 1 | (100) | (6.5) | (2.5) | (2,200/300) | (3.0) | | |
| Comparative | P-8 | PAG9 | Q-1 | PGMEA/GBL | F-1 | 15 | 2.3 |
| Example 2 | (100) | (6.5) | (2.5) | (2,200/300) | (3.0) | | |

The evaluation conducted for the resist patterns formed through the EUV exposure revealed that all the radiation-sensitive resin compositions of Examples had good sensitivity and CDU performance.

### INDUSTRIAL APPLICABILITY

According to the radiation-sensitive resin composition and the method for forming a resist pattern described above, a resist pattern having good sensitivity to exposure light and superior CDU performance can be formed. Therefore, these can be suitably used for a machining process and the like of a semiconductor device in which micronization is expected to further progress in the future.

1. A radiation-sensitive resin composition comprising:
a resin comprising a structural unit represented by formula (1);
at least onium salt each comprising an organic acid anion moiety and an onium cation moiety; and
a solvent, wherein
at least part of the organic acid anion moiety in the at least one onium salt comprises an iodine-substituted aromatic ring structure, 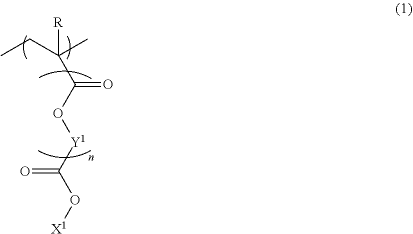
in the formula (1),
R is a hydrogen atom, an alkyl group having 1 to 5 carbon atoms, or a halogenated alkyl group having 1 to 5 carbon atoms, Y¹ is a divalent linking group, and X¹ is an acid-dissociable group, and
n is 0 or 1,
provided that when n is 0, X¹ is represented by the following formula (s1) or (s2), 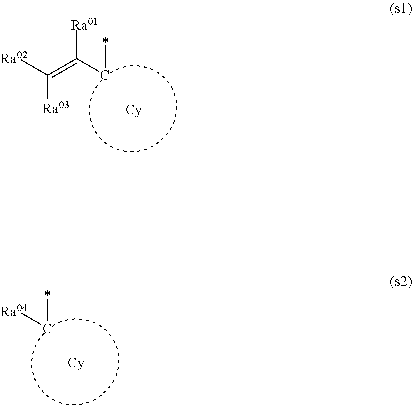
in the formula (s1),
Cy is an aliphatic cyclic group formed with the carbon atom,
Ra⁰¹ to Ra⁰³ each independently represent a hydrogen atom, a substituted or unsubstituted monovalent chain saturated hydrocarbon group having 1 to 10 carbon atoms, or a substituted or unsubstituted monovalent aliphatic cyclic saturated hydrocarbon group having 3 to 20 carbon atoms, and optionally at least two of Ra⁰¹ to Ra⁰³ taken together represent an aliphatic cyclic structure, provided that the aliphatic cyclic structure does not form a crosslinked structure;
in the formula (s2),
Cy is as defined as in the formula (s1), and
Ra⁰⁴ is a substituted or unsubstituted aromatic hydrocarbon group;
in the formula (s1) and formula (s2), each * represents a bond with the oxygen atom in the formula (1).

2. The radiation-sensitive resin composition according to claim 1, wherein the at least one onium salt is at least one member selected from the group consisting of:
a radiation-sensitive acid generator comprising the organic acid anion moiety and the onium cation moiety, and
an acid diffusion controlling agent comprising the organic acid anion moiety and the onium cation moiety and generating an acid having a pKa higher than a pKa of an acid to be generated from the radiation-sensitive acid generator through irradiation with radiation, and
the organic acid anion moiety in the radiation-sensitive acid generator the organic acid anion moiety in the acid diffusion controlling agent, or both comprises the iodine-substituted aromatic ring structure.

3. The radiation-sensitive resin composition according to claim 1, wherein the organic acid anion moiety comprises at least one anion selected from the group consisting of a sulfonate anion, a carboxylate anion, and a sulfonimide anion.

4. The radiation-sensitive resin composition according to claim 1, wherein n is 1, and X¹ in the formula (1) is represented by the formula (s1) or (s2), 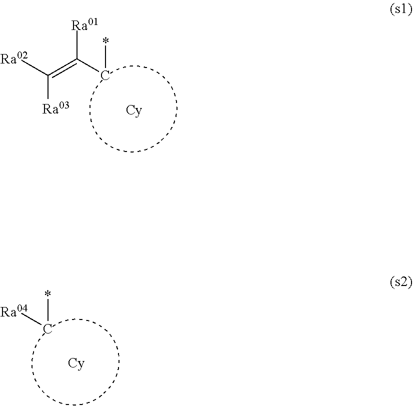
in the formula (s1),
Cy is an aliphatic cyclic group formed with the carbon atom,
Ra⁰¹ to Ra⁰³ each independently represent a hydrogen atom, a substituted or unsubstituted monovalent chain saturated hydrocarbon group having 1 to 10 carbon atoms, or a substituted or unsubstituted monovalent aliphatic cyclic saturated hydrocarbon group having 3 to 20 carbon atoms, and optionally at least two of Ra⁰¹ to Ra⁰³ taken together represent an aliphatic cyclic structure, provided that the aliphatic cyclic structure does not form a crosslinked structure;
in the formula (s2),
Cy is as defined as in the formula (s1), and
Ra⁰⁴ is a substituted or unsubstituted aromatic hydrocarbon group;
in the formula (s1) and formula (s2), each * represents a bond with the oxygen atom in the formula (1).

5. The radiation-sensitive resin composition according to claim 1, wherein the resin further comprises a structural unit having a phenolic hydroxy group.

6. The radiation-sensitive resin composition according to claim 1, wherein the resin further comprises a structural unit comprising at least one structure selected from the group consisting of a lactone structure, a cyclic carbonate structure, and a sultone structure.

7. The radiation-sensitive resin composition according to claim 1, further comprising a high-fluorine-containing resin that is higher in mass content of fluorine atoms than the resin.

8. A method for forming a pattern, the method comprising:
directly or indirectly applying the radiation-sensitive resin composition according to claim 1 on a substrate to form a resist film;
exposing the resist film to light; and
developing the exposed resist film with a developer.

9. The method for forming a pattern according to claim 8, wherein the resist film is exposed to an extreme ultraviolet ray or an electron beam.
